SEQUENCE LISTING (46 sequences)

SEQ ID NO: 1 - protein, 22 aa, Artificial Sequence
  GMCSF signal peptide
MLLLVTSLLLCELPHPAFLLIP

SEQ ID NO: 2 - DNA, 51 bp, Artificial Sequence
  DNA sequence encoding SEQ ID NO 1
atgttgctgctggttacatctctgctgctgtgcgagctgccccatcctgcc

SEQ ID NO: 3 - protein, 116 aa, Artificial Sequence
  JML-1 heavy chain variable domain (VH)
KVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSS

SEQ ID NO: 4 - DNA, 348 bp, Artificial Sequence
  DNA sequence encoding SEQ ID NO 3
aaggtgcagctgctggaatctggcggaggactggttcagcctggcagaagcctgagactgtcttgtgccgccagcggcttcaccttcgacgattatggcatgcactgggtccgacaggcccctggcaaaggacttgaatgggtgtccggcatcagctggaacagcggctctatcggctacgccgattccgtgaagggcagattcaccatcagccgggacaacagcaagaacaccctgtacctgcagatgaacagcctgagagccgaggacaccgccgtgtactattgtgctagaggcggccagaccatcgacatctggggacagggaaccatggtcaccgtttctagc

SEQ ID NO: 5 - protein, 15 aa, Artificial Sequence
  4(GS)x3 linker
GGGGSGGGGSGGGGS

SEQ ID NO: 6 - DNA, 45 bp, Artificial Sequence
  DNA sequence encoding SEQ ID NO 5
ggaggcggaggttctggcggcggaggaagtggtggcggaggctct

SEQ ID NO: 7 - protein, 107 aa, Artificial Sequence
  JML-1 light chain variable domain (VL)
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIK

SEQ ID NO: 8 - DNA, 321 bp, Artificial Sequence
  DNA sequence encoding SEQ ID NO 7
gatatccagatgacacagagccccagcagcctgtctgcctctgtgggagacagagtgaccatcacctgtagagccagccagagcatcagcagctacctgaactggtatcagcaaaagcccggcaaggcccctaaactgctgatctacgctgcctccagtctgcagagcggagtgcctagcagattttctggctctggcagcggcaccgacttcaccctgaccatatctagcctgcagcctgaggacttcgccacctactactgccagcagagctacagcacccctttcacatttggccctggcaccaaggtggacatcaaa

SEQ ID NO: 9 - protein, 12 aa, Artificial Sequence
  IgG4 hinge domain
ESKYGPPCPPCP

SEQ ID NO: 10 - DNA, 36 bp, Artificial Sequence
  DNA sequence encoding SEQ ID NO 9
gagtctaagtacggaccgccttgtcctccttgtcca

SEQ ID NO: 11 - protein, 32 aa, Artificial Sequence
  IgG3 hinge
ELKTPLGDTTHTCPRCPEPKSCDTPPPCPRCP

SEQ ID NO: 12 - DNA, 96 bp, Artificial Sequence
  DNA sequence encoding SEQ ID NO 11
gagctgaaaacccctctgggcgacaccacacacacatgccctagatgtccggaacccaagagctgcgatacccccccaccttgccccagatgcccc

SEQ ID NO: 13 - protein, 28 aa, Artificial Sequence
  CD28 transmembrane domain
MFWVLVVVGGVLACYSLLVTVAFIIFWV

SEQ ID NO: 14 - DNA, 84 bp, Artificial Sequence
  DNA sequence encoding SEQ ID NO 13
atgttttgggtgctggtggtcgtgggcggagtgctggcctgttacagcctgctcgtgaccgtggccttcatcatcttttgggtc

SEQ ID NO: 15 - protein, 41 aa, Artificial Sequence
  CD28 costimulatory domain
RSKRSRGGHSDYMNMTPRRPGPTRKHYQPYAPPRDFAAYRS

SEQ ID NO: 16 - DNA, 123 bp, Artificial Sequence
  DNA sequence encoding SEQ ID NO 15
cgcagcaagcggagcagaggcggccacagcgactacatgaacatgacccctagacggcctggccccaccagaaagcactaccagccctacgcccctccccgggactttgccgcctacagaagc

SEQ ID NO: 17 - protein, 42 aa, Artificial Sequence
  4-1BB costimulatory domain
KRGRKKLLYIFKQPFMRPVQTTQEEDGCSCRFPEEEEGGCEL

SEQ ID NO: 18 - DNA, 126 bp, Artificial Sequence
  DNA sequence encoding SEQ ID NO 17
aagcggggcagaaagaagctgctgtacatctttaagcagcccttcatgcggcccgtgcagaccacccaggaagaggacggctgctcctgcagattccccgaggaagaagaaggcggctgcgagctg

SEQ ID NO: 19 - protein, 112 aa, Artificial Sequence
  CD3zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - DNA, 336 bp, Artificial Sequence
  DNA sequence encoding SEQ ID NO 19
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 21 - protein, 24 aa, Artificial Sequence
  T2A ribosomal skipping sequence
LEGGGEGRGSLLTCGDVEENPGPR

SEQ ID NO: 22 - DNA, 72 bp, Artificial Sequence
  DNA sequence encoding SEQ ID NO 21
ctcgagggcggcggagagggcagaggaagtcttctaacatgcggtgacgtggaggagaatcccggccctagg

SEQ ID NO: 23 - protein, 335 aa, Artificial Sequence
  EGFRt
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 24 - DNA, 1008 bp, Artificial Sequence
  DNA sequence encoding SEQ ID NO 23
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 25 - protein, 238 aa, Artificial Sequence
  JML-1 scFv
KVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIK

SEQ ID NO: 26 - DNA, 714 bp, Artificial Sequence
  DNA sequence encoding SEQ ID NO 25
aaggtgcagctgctggaatctggcggaggactggttcagcctggcagaagcctgagactgtcttgtgccgccagcggcttcaccttcgacgattatggcatgcactgggtccgacaggcccctggcaaaggacttgaatgggtgtccggcatcagctggaacagcggctctatcggctacgccgattccgtgaagggcagattcaccatcagccgggacaacagcaagaacaccctgtacctgcagatgaacagcctgagagccgaggacaccgccgtgtactattgtgctagaggcggccagaccatcgacatctggggacagggaaccatggtcaccgtttctagcggaggcggaggttctggcggcggaggaagtggtggcggaggctctgatatccagatgacacagagccccagcagcctgtctgcctctgtgggagacagagtgaccatcacctgtagagccagccagagcatcagcagctacctgaactggtatcagcaaaagcccggcaaggcccctaaactgctgatctacgctgcctccagtctgcagagcggagtgcctagcagattttctggctctggcagcggcaccgacttcaccctgaccatatctagcctgcagcctgaggacttcgccacctactactgccagcagagctacagcacccctttcacatttggccctggcaccaaggtggacatcaaa

SEQ ID NO: 27 - protein, 453 aa, Artificial Sequence
  full-length CAR with IgG4 hinge and CD28 costimulatory domain
MLLLVTSLLLCELPHPAFLLIPKVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKESKYGPPCPPCPMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - DNA, 714 bp, Artificial Sequence; identical to SEQ ID NO: 26
  DNA sequence encoding SEQ ID NO 27

SEQ ID NO: 29 - protein, 454 aa, Artificial Sequence
  full-length CAR with IgG4 hinge and 4-1BB costimulatory domain
MLLLVTSLLLCELPHPAFLLIPKVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - DNA, 1362 bp, Artificial Sequence
  DNA sequence encoding SEQ ID NO 29
atgttgctgctggttacatctctgctgctgtgcgagctgccccatcctgcctttctgctgatccctaaggtgcagctgctggaatctggcggaggactggttcagcctggcagaagcctgagactgtcttgtgccgccagcggcttcaccttcgacgattatggcatgcactgggtccgacaggcccctggcaaaggacttgaatgggtgtccggcatcagctggaacagcggctctatcggctacgccgattccgtgaagggcagattcaccatcagccgggacaacagcaagaacaccctgtacctgcagatgaacagcctgagagccgaggacaccgccgtgtactattgtgctagaggcggccagaccatcgacatctggggacagggaaccatggtcaccgtttctagcggaggcggaggttctggcggcggaggaagtggtggcggaggctctgatatccagatgacacagagccccagcagcctgtctgcctctgtgggagacagagtgaccatcacctgtagagccagccagagcatcagcagctacctgaactggtatcagcaaaagcccggcaaggcccctaaactgctgatctacgctgcctccagtctgcagagcggagtgcctagcagattttctggctctggcagcggcaccgacttcaccctgaccatatctagcctgcagcctgaggacttcgccacctactactgccagcagagctacagcacccctttcacatttggccctggcaccaaggtggacatcaaagagtctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 31 - protein, 473 aa, Artificial Sequence
  full-length CAR with IgG3 hinge and CD28 costimulatory domain
MLLLVTSLLLCELPHPAFLLIPKVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKELKTPLGDTTHTCPRCPEPKSCDTPPPCPRCPMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - DNA, 1419 bp, Artificial Sequence
  DNA sequence encoding SEQ ID NO 31
atgttgctgctggttacatctctgctgctgtgcgagctgccccatcctgcctttctgctgatccctaaggtgcagctgctggaatctggcggaggactggttcagcctggcagaagcctgagactgtcttgtgccgccagcggcttcaccttcgacgattatggcatgcactgggtccgacaggcccctggcaaaggacttgaatgggtgtccggcatcagctggaacagcggctctatcggctacgccgattccgtgaagggcagattcaccatcagccgggacaacagcaagaacaccctgtacctgcagatgaacagcctgagagccgaggacaccgccgtgtactattgtgctagaggcggccagaccatcgacatctggggacagggaaccatggtcaccgtttctagcggaggcggaggttctggcggcggaggaagtggtggcggaggctctgatatccagatgacacagagccccagcagcctgtctgcctctgtgggagacagagtgaccatcacctgtagagccagccagagcatcagcagctacctgaactggtatcagcaaaagcccggcaaggcccctaaactgctgatctacgctgcctccagtctgcagagcggagtgcctagcagattttctggctctggcagcggcaccgacttcaccctgaccatatctagcctgcagcctgaggacttcgccacctactactgccagcagagctacagcacccctttcacatttggccctggcaccaaggtggacatcaaagagctgaaaacccctctgggcgacaccacacacacatgccctagatgtccggaacccaagagctgcgatacccccccaccttgccccagatgccccatgttttgggtgctggtggtcgtgggcggagtgctggcctgttacagcctgctcgtgaccgtggccttcatcatcttttgggtccgcagcaagcggagcagaggcggccacagcgactacatgaacatgacccctagacggcctggccccaccagaaagcactaccagccctacgcccctccccgggactttgccgcctacagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 33 - protein, 474 aa, Artificial Sequence
  full-length CAR with IgG3 hinge and 4-1BB costimulatory domain
MLLLVTSLLLCELPHPAFLLIPKVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKELKTPLGDTTHTCPRCPEPKSCDTPPPCPRCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - DNA, 1422 bp, Artificial Sequence
  DNA sequence encoding SEQ ID NO 33
atgttgctgctggttacatctctgctgctgtgcgagctgccccatcctgcctttctgctgatccctaaggtgcagctgctggaatctggcggaggactggttcagcctggcagaagcctgagactgtcttgtgccgccagcggcttcaccttcgacgattatggcatgcactgggtccgacaggcccctggcaaaggacttgaatgggtgtccggcatcagctggaacagcggctctatcggctacgccgattccgtgaagggcagattcaccatcagccgggacaacagcaagaacaccctgtacctgcagatgaacagcctgagagccgaggacaccgccgtgtactattgtgctagaggcggccagaccatcgacatctggggacagggaaccatggtcaccgtttctagcggaggcggaggttctggcggcggaggaagtggtggcggaggctctgatatccagatgacacagagccccagcagcctgtctgcctctgtgggagacagagtgaccatcacctgtagagccagccagagcatcagcagctacctgaactggtatcagcaaaagcccggcaaggcccctaaactgctgatctacgctgcctccagtctgcagagcggagtgcctagcagattttctggctctggcagcggcaccgacttcaccctgaccatatctagcctgcagcctgaggacttcgccacctactactgccagcagagctacagcacccctttcacatttggccctggcaccaaggtggacatcaaagagctgaaaacccctctgggcgacaccacacacacatgccctagatgtccggaacccaagagctgcgatacccccccaccttgccccagatgccccatgttttgggtgctggtggtcgtgggcggagtgctggcctgttacagcctgctcgtgaccgtggccttcatcatcttttgggtcaagcggggcagaaagaagctgctgtacatctttaagcagcccttcatgcggcccgtgcagaccacccaggaagaggacggctgctcctgcagattccccgaggaagaagaaggcggctgcgagctgagagtgaagttcagcagatccgccgacgcccctgcctatcagcagggccagaaccagctatacaacgagctgaacctgggcagacgggaagagtacgacgtgctggacaagagaagaggccgggaccctgagatgggcggaaagcccagaagaaagaacccccaggaaggcctgtataacgaactgcagaaagacaagatggccgaggcctacagcgagatcggaatgaagggcgagcggcggagaggcaagggccacgatggactgtatcagggcctgagcaccgccaccaaggacacctatgacgccctgcacatgcaggccctgccccctaga

SEQ ID NO: 35 - protein, 272 aa, Artificial Sequence
  extracellular domain with IgG4 hinge
MLLLVTSLLLCELPHPAFLLIPKVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKESKYGPPCPPCP

SEQ ID NO: 36 - DNA, 816 bp, Artificial Sequence
  DNA sequence encoding SEQ ID NO 35
atgttgctgctggttacatctctgctgctgtgcgagctgccccatcctgcctttctgctgatccctaaggtgcagctgctggaatctggcggaggactggttcagcctggcagaagcctgagactgtcttgtgccgccagcggcttcaccttcgacgattatggcatgcactgggtccgacaggcccctggcaaaggacttgaatgggtgtccggcatcagctggaacagcggctctatcggctacgccgattccgtgaagggcagattcaccatcagccgggacaacagcaagaacaccctgtacctgcagatgaacagcctgagagccgaggacaccgccgtgtactattgtgctagaggcggccagaccatcgacatctggggacagggaaccatggtcaccgtttctagcggaggcggaggttctggcggcggaggaagtggtggcggaggctctgatatccagatgacacagagccccagcagcctgtctgcctctgtgggagacagagtgaccatcacctgtagagccagccagagcatcagcagctacctgaactggtatcagcaaaagcccggcaaggcccctaaactgctgatctacgctgcctccagtctgcagagcggagtgcctagcagattttctggctctggcagcggcaccgacttcaccctgaccatatctagcctgcagcctgaggacttcgccacctactactgccagcagagctacagcacccctttcacatttggccctggcaccaaggtggacatcaaagagtctaagtacggaccgccctgccccccttgccct

SEQ ID NO: 37 - protein, 292 aa, Artificial Sequence
  extracellular domain with IgG3 hinge
MLLLVTSLLLCELPHPAFLLIPKVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKELKTPLGDTTHTCPRCPEPKSCDTPPPCPRCP

SEQ ID NO: 38 - DNA, 876 bp, Artificial Sequence
  DNA sequence encoding SEQ ID NO 37
atgttgctgctggttacatctctgctgctgtgcgagctgccccatcctgcctttctgctgatccctaaggtgcagctgctggaatctggcggaggactggttcagcctggcagaagcctgagactgtcttgtgccgccagcggcttcaccttcgacgattatggcatgcactgggtccgacaggcccctggcaaaggacttgaatgggtgtccggcatcagctggaacagcggctctatcggctacgccgattccgtgaagggcagattcaccatcagccgggacaacagcaagaacaccctgtacctgcagatgaacagcctgagagccgaggacaccgccgtgtactattgtgctagaggcggccagaccatcgacatctggggacagggaaccatggtcaccgtttctagcggaggcggaggttctggcggcggaggaagtggtggcggaggctctgatatccagatgacacagagccccagcagcctgtctgcctctgtgggagacagagtgaccatcacctgtagagccagccagagcatcagcagctacctgaactggtatcagcaaaagcccggcaaggcccctaaactgctgatctacgctgcctccagtctgcagagcggagtgcctagcagattttctggctctggcagcggcaccgacttcaccctgaccatatctagcctgcagcctgaggacttcgccacctactactgccagcagagctacagcacccctttcacatttggccctggcaccaaggtggacatcaaagagctgaaaacccctctgggcgacaccacacacacatgccctagatgtccggaacccaagagctgcgatacccccccaccttgccccagatgcccc

SEQ ID NO: 39 - protein, 153 aa, Artificial Sequence
  intracellular domain with CD28 costimulatory domain
RSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 459 bp, Artificial Sequence
  DNA sequence encoding SEQ ID NO 39
cgcagcaagcggagcagaggcggccacagcgactacatgaacatgacccctagacggcctggccccaccagaaagcactaccagccctacgcccctccccgggactttgccgcctacagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 41 - protein, 154 aa, Artificial Sequence
  intracellular domain with 4-1BB costimulatory domain
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - DNA, 462 bp, Artificial Sequence
  DNA sequence encoding SEQ ID NO 41
aaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 43 - DNA, 225 bp, Artificial Sequence
  left IR/DR segment
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacact

SEQ ID NO: 44 - DNA, 225 bp, Artificial Sequence
  right IR/DR segment
agtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 45 - protein, 340 aa, Artificial Sequence
  Sleeping Beauty amino acid sequence
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 46 - DNA, 1359 bp, Artificial Sequence
  DNA sequence encoding full-length siglec-6
atgcagggagcccaggaagcctccgcctcagagatgctaccgctgctgctgcccctgctgtgggcaggggccctggctcaggagcggagattccagctggaggggccagagtcactgacggtgcaggagggtctgtgcgtcctcgtaccctgcagattgcccactacccttccagcctcgtactatggttatggctactggttcctggaaggggctgatgttccagtggccacaaacgacccagacgaagaagtgcaggaggagacccggggccgattccacctcctctgggatcccagaaggaagaactgctccctgagcatcagagatgcccggaggagggacaatgctgcatacttctttcggttgaagtccaaatggatgaaatacggttatacatcttccaagctctctgtgcgtgtgatggccctgacccacaggcccaacatctccatcccagggaccctggagtctggccatcccagcaatctgacctgctctgtgccctgggtctgtgagcaggggacgccccccatcttctcctggatgtcagctgcccccacctccctgggccccaggaccacccagtcctcggtgctcacaatcaccccacggccccaggaccacagcaccaacctcacctgtcaggtgacgttccctggagccggtgtgaccatggagagaaccatccagctcaatgtctcctatgctccacagaaagtggccatcagcatcttccaaggaaacagcgcagccttcaaaatcctgcaaaacacctcgtccctccctgtcctggagggccaggctctgcggctgctctgtgatgctgacggcaacccccctgcacacctgagctggttccagggcttccccgccctgaacgccacccccatctccaataccggggtcctggagctgcctcaagtagggtctgcagaagaaggagatttcacctgccgtgctcagcatcctctgggctccctgcaaatctctctgagtctctttgtgcattggaaaccagaaggcagggctggtggtgtcctgggagcagtctggggagctagcatcacaaccctggttttcctctgtgtttgcttcatcttcagagtgaagactagaaggaagaaagcagcccagccagtgcaaaacacggatgatgtgaaccccgtcatggtctcaggctccaggggtcatcagcaccagttccagacaggcatagtttcagaccaccctgctgaggctggccccatctcagaagatgagcaggagctccactacgctgtcctacacttccacaaggtgcaacctcaggaaccaaaggtcaccgacactgagtactcagaaatcaagatacacaag

(19) United States
(12) Patent Application Publication
(10) Pub. No.: US 2023/0287129 A1
(43) Pub. Date: Sep. 14, 2023

(54) SIGLEC-6-BINDING POLYPEPTIDES

(71) Applicant: JULIUS-MAXIMILIANS-UNIVERSITÄT WÜRZBURG, Würzburg (DE)

(72) Inventors: Michael HUDECEK, Höchberg (DE); Hardikkumar JETANI, Amsterdam (NL)

(21) Appl. No.: 18/019,238

(22) PCT Filed: Aug. 9, 2021
(86) PCT No.: PCT/EP2021/072160

§ 371 Date: Feb. 1, 2023

(30) Foreign Application Priority Data
Aug. 10, 2020 (EP) 20190301.0
Jul. 2, 2021 (EP) 21183475.9

(51) Int. Cl. C07K 16/28; C07K 14/705; C07K 14/725; C12N 15/86; A61K 35/17; A61P 35/02

(52) U.S. Cl. CPC C07K 16/2851; C07K 14/70521; C07K 14/7051; C12N 15/86; A61K 35/17; A61P 35/02

(57) ABSTRACT

The present invention relates to a siglec-6-binding polypeptide that comprises or consists of an antibody or a fragment thereof binding siglec-6 or comprises or consists of a siglec-6-binding chimeric antigen receptor (CAR), a polynucleotide encoding the siglec-6-binding polypeptide, an expression vector comprising the polynucleotide, an immune cell comprising the polypeptide, polynucleotide or expression vector, a method for producing immune cells and a pharmaceutical composition comprising immune cells. The immune cells and the pharmaceutical composition of the present invention may be used in methods for treating a disease, such as cancer, in a patient.

### FIELD OF INVENTION

The present invention relates to a siglec-6-binding polypeptide that comprises or consists of an antibody or a fragment thereof binding siglec-6 or a siglec-6-binding chimeric antigen receptor (CAR), a polynucleotide encoding the siglec-6-binding polypeptide, an expression vector comprising the polynucleotide, an immune cell comprising the polypeptide, polynucleotide or expression vector, a method for producing such immune cells and a pharmaceutical composition comprising such immune cells. The immune cells and the pharmaceutical composition of the present invention may be used in methods for treating a disease in a patient.

### BACKGROUND

T-cells endowed with synthetic chimeric antigen receptor (CAR) have shown durable remissions in B-cell and plasma cell malignancies [1-4] and there is a quest for targeting other hematological and solid cancers with CAR T-cells. Acute Myeloid Leukemia (AML) is an entity with unmet medical need and requires novel curative therapies. Although several preclinical studies show feasibility of targeting AML with CAR-T cells, the identification of a CAR target antigen with acceptable safety profile remains challenging [5]. This is mainly due to the expression of previously identified candidate CAR target antigens in AML on normal hematopoietic stem and progenitor cells (HSC/P) [6,7]. Therefore, there is a strong desire for a novel CAR target that is not expressed by HSC/P and that has no or minimal expression on other healthy cells and tissues.

CAR-T cells targeting B-cell and plasma cell malignancies have shown unprecedented clinical responses in patients with multiple prior lines of treatments and advanced hematologic malignancies [1-4], raising interest in using CAR T-cell therapy in AML. Because CAR T-cells target cell surface molecules, it is desirable that a target antigen is uniformly expressed on tumor cells and has minimal or even absent expression on healthy cells and tissues. This prerequisite is challenging for CAR-T therapy in AML because most of the candidate antigens that have previously been proposed are expressed by healthy HSC/P and by innate immune cells [20]. Therefore, directing CAR T-cells against these antigens is anticipated to lead to undesired toxicities including reduction or ablation of healthy hematopoiesis. Indeed, CAR T-cells against myeloid-lineage antigens, e.g. CD123, CD33 has been shown to be myeloablative, and to necessitate allogeneic hematopoietic stem cell transplantation (alloHSCT) to reconstitute normal hematopoiesis [19, 22]. Of note, the clinical use of CD123-specific CAR T-cells has resulted in significant and unexpected toxicity and fatal severe adverse events in a first-in-man clinical trial that employed an allogeneic CD123-CAR T-cell product and as a consequence, this trial had been placed on hold [21]. In order to prevent elimination of normal HSC/P, Kim et al. suggested that gene editing using CRISPR/Cas9 to knockdown CD33 in donor HSC/P cells may prevent elimination of HSC/P by CD33-CAR T-cells [22]. However, clinical implementation of this strategy—that entails the administration of gene-edited HSC (in addition to gene-modified CD33-CAR T-cells)—would be extremely laborious, complex, and expensive. In addition, the use of gene-edited HSCs entails a substantially greater risk for undesired genotoxicity, including a risk for malignant transformation.

Additionally, other potential CAR targets of interest in AML include FLT3, CLL-1, CD44v6, CD7, folate receptor p, Lewis-y antigen. The inventors have previously shown that targeting FLT3 with CAR T-cells in high-risk FLT3-ITD+AML can induce complete remissions in mouse xenografts and that the anti-leukemia efficacy of FLT3 CAR-T cells can be enhanced by FLT3-inhibitors [23]. However, FLT3 is also expressed on HSC/P and FLT3-CAR T-cells treatment is anticipated to induce myeloablation [23]. C-type lectin-like molecule-1 (CLL-1) is expressed by AML blasts and is also present on lung and gastrointestinal epithelial cells [24], and the inventors observed CLL-1-expression on HSC/P, suggesting the potential for severe toxicities if targeted by CLL-1-specific CAR T-cells. Although CD44v6 is absent on HSC/P, its expression in vital cells and tissues such as keratinocytes, oral mucosa and monocytes may lead to lethal toxicities when targeted by CD44v6-CAR T-cells [25]. CD7 is expressed only by ˜30% AML patients and at high-levels on T-cells [26], requiring CD7 knockout from T-cells to manufacture anti-CD7-CAR T-cells, further making clinical application complex.

Sialic-acid-binding immunoglobulin-like lectins (siglecs) are a immunoglobulin superfamily of cell surface receptors that are expressed mainly by leukocytes and are associated with inhibitory signaling in human immune cells [8]. Notably, siglec-2 (CD22) and siglec-3 (CD33) are member of siglec superfamily and of interest as CAR target antigens in hematological malignancies such as B-cell Acute Lymphoblastic Leukemia (B-ALL) and AML, respectively. Encouragingly, CD22-targeted CAR T-cells have shown complete remissions in relapsed/refractory (R/R) B-ALL patients [9], indicating that targeting siglecs with favorable expression profile can induce leukemia remissions and potentially cure patients.

Baskar et al. generated monoclonal antibodies (mAbs) from a post-allogeneic hematopoietic stem cell transplantation (alloHSCT) repertoire that potentially contributed to the graft-versus leukemia (GVL) response in a chronic lymphocytic leukemia (CLL) patient [10]. Subsequent target discovery analyses revealed mAb ‘JML-1’ as a candidate which binds and recognizes human siglec-6 protein [11]. Siglec-6 belongs to CD33-related siglec subfamily and its structure closely relates to siglec-3 (CD33). Siglec-6 consists of three extracellular immunoglobulin (Ig) domains and two intracellular immunoreceptor tyrosine-based inhibition motifs (ITIM) motifs [12-14]. Due to these ITIM motifs, siglec-6 is thought to serve as regulator of activating pathways like other CD33-related siglecs [13]. Siglec-6-expression is reported in primary B-cells [10,12] and aberrantly in CLL [10,11] and in MALT lymphoma [15]. Siglec-6 is also known to be expressed on placenta [12,16] and human mast cells [17,18]. However, unlike other siglec proteins, it is absent on NK cells, T cells, neutrophils, macrophages and monocytes [13].

In view of the above, there still remains the critical need for novel therapies that provide a safe and effective treatment for leukemia and lymphoma, and especially AML, CLL, MALT lymphoma and clonal mast cell diseases.

### DESCRIPTION OF INVENTION

The present invention aims to overcome the unmet clinical needs by providing an improved composition for therapeutic treatment of patients.

The inventors demonstrate siglec-6-expression on primary AML blasts derived from newly diagnosed and relapsed/refractory AML patients. Intriguingly, the inventors also demonstrate siglec-6-expression on AML leukemic stem cells (LSCs). Human CD4⁺ and CD8+ T-cells were equipped to express a siglec-6-specific CAR with a targeting domain derived from the fully human JML-1 IgG1 mAb (JML-1-CAR). The anti-leukemia reactivity of AML patient and HD derived JML-1-CAR T-cells against primary ‘bulk’ AML blasts, AML leukemic stem cells and AML cell lines was assessed and demonstrated. Further, the inventors evaluated the expression of siglec-6 on normal hematopoietic stem/progenitor cells (HSC/P) and mature peripheral blood cells, in order to assess potential on-target off-tumor hematologic toxicity mediated by JML-1-CAR T-cells. The inventors show that siglec-6 is not expressed on normal HSC/P and demonstrate that normal HSC/P are not recognized by JML-1-CAR T-cells. High levels of siglec-6 on malignant B-CLL cells from treatment-naïve CLL patients and in healthy B-cells were confirmed and anti-leukemia activity of JML-1 CAR-T cells against CLL demonstrated.

The present application plausibly shows for the first time that a therapy using immune cells binding to siglec-6, such as immune cells comprising a CAR binding to siglec-6, is efficacious. Such therapy involves the elimination of siglec-6 expressing cells.

Furthermore, the present application confirms that siglec-6 is not expressed on non-cancerous hematopoietic stem/progenitor cells (HSC/P), suggesting targeting siglec-6 with immune cells, such as CAR-T cells, could be a safe approach in treating cancer, such as AML, and may not require subsequent alloHSCT. The application therefore for the first time presents a treatment using immune cells binding to siglec-6 that does not involve elimination of non-cancerous HSC/P and consequently obviates the need to perform alloHSCT after such immunotherapy. Moreover, the use of immune cells binding siglec-6 can obviate the need to deplete said immune cells after the treatment.

Accordingly, the present invention provides the following preferred embodiments:

[1] A siglec-6-binding polypeptide that comprises or consists of an antibody or a fragment thereof binding siglec-6 or that comprises or consists of a chimeric antigen receptor (CAR).

[2] The siglec-6-binding polypeptide according to [1] that comprises or consists of an antibody or a fragment thereof binding siglec-6.

[3] The siglec-6-binding polypeptide according to [1] or [2] that is at least bispecific.

[4] The siglec-6-binding polypeptide according to [2] or [3] that comprises or consists of a first antibody or a fragment thereof binding siglec-6 and a second antibody or fragment thereof binding to a target other than siglec-6, optionally connected to each other via a linker.

[5] The siglec-6-binding polypeptide according to any one of [2]-[4], wherein the antibody or a fragment thereof binding siglec-6 is represented by an amino acid sequence shown in SEQ ID NO: 25 or by an amino acid sequence having at least 90% identity to an amino acid sequence shown in SEQ ID NO: 25.

[6] The siglec-6-binding polypeptide according to [4] or [5] that is capable of binding to an immune cell, such as a T cell or an NK cell, preferably to a T cell.

[7] The siglec-6-binding polypeptide according to any one of [4]-[6] that additionally binds to CD3, such as CD3zeta or CD3epsilon, preferably CD3zeta.

[8] The siglec-6-binding polypeptide according to any one of [4]-[7] that is capable of recruiting an immune cell, such as a T cell or an NK cell, preferably a T cell, to a target cell expressing siglec-6 on its surface.

[9] The siglec-6-binding polypeptide according to any one of [2]-[5] that is conjugated to a drug.

[10] The siglec-6-binding polypeptide according to [9], wherein the drug is a toxin.

[11] The siglec-6-binding polypeptide according to [1] or [3] that comprises or consists of a siglec-6-binding CAR.

[12] The siglec-6-binding polypeptide according to [11], wherein the CAR comprises at least one extracellular ligand binding domain, a transmembrane domain and at least one intracellular signalling domain.

[13] The siglec-6-binding polypeptide according to [12], wherein said extracellular ligand binding domain comprises a siglec-6-binding element.

[14] The siglec-6-binding polypeptide according to [13], wherein the siglec-6-binding element is represented by an amino acid sequence shown in SEQ ID NO: 25 or by an amino acid sequence having at least 90% identity to an amino acid sequence shown in SEQ ID NO: 25.

[15] The siglec-6-binding polypeptide according to any one of [12]-[14], wherein the extracellular ligand binding domain comprises a spacer domain, such as a spacer domain from CD8a, IgG3 or IgG4.

[16] The siglec-6-binding polypeptide according to any one of [12]-[15], wherein said transmembrane domain comprises a CD28 transmembrane domain, preferably represented by an amino acid sequence shown in SEQ ID NO: 13 or by an amino acid sequence having at least 90% identity to an amino acid sequence shown in SEQ ID NO: 13.

[17] The siglec-6-binding polypeptide according to any one of [12]-[16], wherein said intracellular signalling domain comprises a costimulatory domain and a CD3 zeta domain, wherein the costimulatory domain is preferably a CD28 cytoplasmic domain or a 4-1BB costimulatory domain.

[18] The siglec-6-binding polypeptide according to [17], wherein the costimulatory domain is a CD28 cytoplasmic domain.

[19] The siglec-6-binding polypeptide according to any one of [17] or [18], wherein the CD28 cytoplasmic domain is represented by an amino acid sequence shown in SEQ ID NO: 15 or by an amino acid sequence having at least 90% identity to an amino acid sequence shown in SEQ ID NO:15.

[20] The siglec-6-binding polypeptide according to [17], wherein the 4-1BB costimulatory domain is represented by an amino acid sequence shown in SEQ ID NO: 17 or by an amino acid sequence having at least 90% identity to an amino acid sequence shown in SEQ ID NO: 17.

[21] The siglec-6-binding polypeptide according to any one of [17]-[20], wherein the CD3 zeta domain is represented by an amino acid sequence shown in SEQ ID NO: 19 or by an amino acid sequence having at least 90% identity to an amino acid sequence shown in SEQ ID NO: 19.

[22] The siglec-6-binding polypeptide according to any one of [12]-[19] and [21], wherein the polypeptide comprises an amino acid sequence shown in any one of SEQ ID NOs: 27, 29, 31 or 33 or an amino acid sequence having at least 90% identity to an amino acid sequence shown in any one of SEQ ID NOs: 27, 29, 31 or 33.

[23] A polynucleotide or set of polynucleotides encoding the siglec-6-binding polypeptide according to any one of the preceding items.

[24] The polynucleotide or set of polynucleotides according to [23], wherein the polynucleotide comprises a nucleotide sequence represented by SEQ ID NO: 26 or a nucleotide sequence having at least 80% identity to nucleotide sequence shown in SEQ ID NO: 26.

[25] The polynucleotide or set of polynucleotides according to [23] or [24], wherein the polynucleotide comprises a nucleotide sequence represented by any one of SEQ ID NO: 28, 30, 32 or 34, or a nucleotide sequence having at least 80% identity to nucleotide sequence shown in any one of SEQ ID NO: 28, 30, 32 or 34.

[26] The polynucleotide according to any one of [23]-[25], wherein the polynucleotide further comprises flanking segments in 5′-direction and in 3′-direction of the polynucleotide encoding the polypeptide.

[27] The polynucleotide according to [26], wherein the flanking segment in 5′-direction is a left inverted repeat/direct repeat (IR/DR) segment and the flanking segment in 3′-direction is a right inverted repeat/direct repeat (IR/DR) segment.

[28] The polynucleotide according to [27], wherein the left IR/DR segment is represented by SEQ ID NO: 43 and right IR/DR segment is represented by SEQ ID NO: 44.

[29] The polynucleotide according to any one of [23]-[28], wherein the polynucleotide comprises a nucleotide sequence of a left IR/DR, a polynucleotide sequence encoding the siglec-6-binding polypeptide and a nucleotide sequence of a right IR/DR.

[30] An expression vector comprising a polynucleotide or set of polynucleotides according to any one of [23]-[29].

[31] The expression vector according to [30] that is a non-viral vector or a viral vector.

[32] The expression vector according to [31] that is a non-viral vector.

[33] The expression vector according to [32], wherein the expression vector is a minimal DNA expression cassette.

[34] The expression vector according to [32] or [33], wherein the expression vector is a transposon donor DNA molecule.

[35] The expression vector according to [34], wherein the transposon donor DNA molecule is a Sleeping Beauty or PiggyBac transposon donor DNA molecule.

[36] The expression vector according to any one of [32]-[35], wherein the expression vector is a minicircle DNA.

[37] The expression vector according to [31] that is a viral vector.

[38] The expression vector according to [37] that that is a lentiviral or gamma-retroviral vector.

[39] An immune cell comprising a siglec-6-binding polypeptide according to any one of [11]-[22] and/or a polynucleotide or set of polynucleotides encoding a siglec-6-binding polypeptide according to any one of [11]-[22] and/or an expression vector comprising a polynucleotide or set of polynucleotides encoding a siglec-6-binding polypeptide according to any one of claims [11]-[22].

[40] The immune cell according to [39], wherein the polynucleotide or set of polynucleotides and/or the vector is expressed.

[41] The immune cell according to any one of the [39]-[40], wherein said immune cell is a lymphocyte.

[42] The immune cell according to [41], wherein said lymphocyte is a T cell or an NK cell.

[43] The immune cell according to [42], wherein said T cell is a CD4⁺ cell or a CD8⁺ cell.

[44] The immune cell according to any one of the [39]-[43], further expressing a detectable marker.

[45] The immune cell according to any one of the claims [39]-[44], wherein said immune cell is a human cell.

[46] Method for producing (recombinant) immune cells, comprising the steps of
(a) isolating immune cells from a blood sample of a subject,
(b) transforming or transducing the immune cells with a polynucleotide according to any one of [23]-[29] or an expression vector according to any one of [30]-[38], and
(c) optionally purifying the transformed or transduced immune cells.

[47] The method according to [46], wherein, in step (b), the immune cells are transformed using 1) a transposable element comprising a polynucleotide according to any one of [23] to [29] and 2) a (polynucleotide encoding a) transposase.

[48] The method according to [47], wherein the transposase is Sleeping Beauty transposase or PiggyBac transposase.

[49] The method according to [48], wherein the Sleeping Beauty transposase is represented by an amino acid sequence shown in SEQ ID NO: 45.

[50] The method according to any one of [47]-[49], wherein the transposable element is integrated into the genome of the immune cells by the action of the transposase.

[51] The method according to any one of [46]-[50], wherein the immune cell is a lymphocyte.

[52] The method according to [51], wherein the lymphocyte is a T cell or an NK cell.

[53] The method according to [52], wherein the T cell is a CD4⁺ cell or a CD8⁺ cell.

[54] The method according to any one of [46]-[53], wherein the subject is a human.

[55] An immune cell obtainable by the method of any one of [46]-[54].

[56] A pharmaceutical composition comprising a plurality of immune cells according to any one of [39]-[45] or of [55], wherein the plurality of immune cells is optionally be a mixture of CD4⁺ and CD8⁺ cells.

[57] The immune cell according to any one of [39]-[45] or of [55], or the pharmaceutical composition according to [56] for use as a medicament.

[58] The immune cell according to any one of [39]-[45] or of [55], or the pharmaceutical composition according to [56] for use in a method of treating cancer, wherein the immune cell or the pharmaceutical composition is to be administered to a subject.

[59] The immune cell or the pharmaceutical composition for use according to [57] or [58], wherein the pharmaceutical composition is to be administered intravenously.

[60] The immune cell or the pharmaceutical composition for use according to any one of [57]-[59], wherein said immune cell is a lymphocyte.

[61] The immune cell or the pharmaceutical composition for use according to [60], wherein said lymphocyte is a T cell or an NK cell.

[62] The immune cell or the pharmaceutical composition for use according to [61], wherein the T cell is a CD4⁺ T cell and/or CD8⁺ T cell.

[63] The immune cell or the pharmaceutical composition for use according to any one of [58]-[62], wherein said subject is a human.

[64] The immune cell or the pharmaceutical composition for use according to any one of [58]-[63], wherein said cancer is a siglec-6 expressing cancer.

[65] The immune cell or the pharmaceutical composition for use according to any one of [58]-[64], wherein said cancer is leukemia.

[66] The immune cell or the pharmaceutical composition for use according to any one of [58]-[65], wherein said cancer is primary acute myeloid leukemia (AML), chronic lymphocytic leukemia (CLL), MALT lymphoma, clonal mast cell disease or thymoma.

[67] The immune cell or the pharmaceutical composition for use according to any one of [58]-[66], wherein the cancer is AML.

[68]. The immune cell or the pharmaceutical composition for use according to any one of [58]-[67], wherein the method of treating cancer involves the elimination of cancer stem cells of said cancer by said immune cells.

[69] The immune cell or the pharmaceutical composition for use according to [68], wherein the cancer stem cells are CD45^{dim} cells, preferably CD45^{dim}CD34⁺ cells, and most preferably CD45^{dim}CD34⁺ CD38-cells.

[70] The immune cell or the pharmaceutical composition for use according to any one of [58]-[69], wherein the method of treating cancer does not involve the elimination of non-cancerous hematopoietic stem or progenitor cells by said immune cells.

[71] The immune cell or the pharmaceutical composition for use according to any one of [68]-[70], further comprising monitoring the elimination of said cancer stem cells and/or of said non-cancerous hematopoietic stem or progenitor cells.

[72] The immune cell or the pharmaceutical composition for use according to any one of [58]-[71], wherein the method of treating cancer does not involve subsequent allogeneic hematopoietic stem cell transplantation, or wherein the subject is a subject having a relapse of the cancer after allogeneic hematopoietic stem cell transplantation.

[73] The immune cell or the pharmaceutical composition for use according to any one of [58]-[72], wherein the method does not involve additional chemotherapy after administration of the immune cells or the pharmaceutical composition and/or after the termination of the therapy with the immune cells or the pharmaceutical composition.

[74] The immune cell or the pharmaceutical composition for use according to any one of [58]-[73], wherein the method of treating cancer does not involve depletion of said immune cells after treatment.

[75] The immune cell or pharmaceutical composition for use according to any one of [58]-[74], wherein the method comprises:
1) determining the expression level of siglec-6 on cancer cells obtained from the subject; followed by
2) administering the immune cell or pharmaceutical composition to the subject.

[76] The immune cell or pharmaceutical composition for use according to [75], wherein the immune cell or pharmaceutical composition is administered in step 2) only if siglec-6 is expressed on said cancer cells.

[77] The immune cell or pharmaceutical composition for use according to any one of [58]-[76], wherein the method involves additional therapy with
(i) a CD70-binding polypeptide that comprises or consists of an antibody or a fragment thereof binding CD70 or that comprises or consists of a chimeric antigen receptor (CAR), or
(ii) an immune cell comprising a CD70-binding polypeptide according to (i) and/or a polynucleotide or set of polynucleotides encoding a CD70-binding polypeptide according to (i) and/or an expression vector comprising a polynucleotide or set of polynucleotides encoding a CD70-binding polypeptide according to (i),
said immune cell being preferably a T-cell such as a CD4⁺ T-cell or CD8⁺-T-cell or an NK-cell.

[78] The immune cell or pharmaceutical composition for use according to [77], wherein the CD70-binding polypeptide comprises or consists of a chimeric antigen receptor (CAR).

[79] The immune cell or pharmaceutical composition for use according to any one of [58]-[78], wherein the method involves additional therapy with
(i) a TIM-3-binding polypeptide that comprises or consists of an antibody or a fragment thereof binding TIM-3 or that comprises or consists of a chimeric antigen receptor (CAR), or
(ii) an immune cell comprising a TIM-3-binding polypeptide according to (i) and/or a polynucleotide or set of polynucleotides encoding a TIM-3-binding polypeptide according to (i) and/or an expression vector comprising a polynucleotide or set of polynucleotides encoding a TIM-3-binding polypeptide according to (i),
said immune cell being preferably a T-cell such as a CD4⁺ T-cell or CD8*-T-cell or an NK-cell.

[80] The immune cell or pharmaceutical composition for use according to [79], wherein the TIM-3-binding polypeptide comprises or consists of a chimeric antigen receptor (CAR).

### BRIEF DESCRIPTION OF THE DRAWINGS

FIG. 1. JML-1-CAR T-cells recognize and eliminate siglec-6⁺ AML cell lines in vitro. (A) Flow cytometric analysis of siglec-6-expression on AML cell lines (U937, MV4;11, MOLM13 and K562). Histograms show staining with anti-siglec-6 mAb (grey) and isotype control antibody (white histograms). Inset numbers state the normalized mean fluorescence intensity (NMFI). (B) Specific cytolytic activity of CD8⁺ JML-1_28z CAR, JML-1_BBz CAR, FLT3_28z CAR, and untransduced (UTD) T-cells against AML cell lines in a luminescence-based assay (4-hour). Assay was performed in triplicate wells with 5,000 target cells/well. Values are presented as mean±s.d. (C) ELISA was performed to detect IFN-γ and IL-2 in supernatant obtained after 24-hour co-culture of CD4⁺ or CD8+JML-1_28z CAR, JML-1_BBz CAR, FLT3_28z CAR or UTD T-cells with target cells. T-cells and target cells were seeded at an effector: target (2:1) in triplicate wells. Values are represented as mean±s.d. (D) Proliferation of CD4⁺ and CD8+JML-1_28z CAR and JML-1_BBz CAR T-cells examined by CFSE dye dilution after 72-hours of co-culture with target cells. Assay was performed in triplicate wells at an effector: target (2:1). Histograms show proliferation of live (7-AAD-) T-cells. No exogenous cytokines were added. Data shown in B-D are representative for results obtained with CAR and control T-cell lines prepared from n >5 healthy donors (HD).

FIG. 2. JML-1-CAR T-cells recognize and eliminate primary AML cells in vitro. (A) Flow cytometric analysis of siglec-6-expression on bulk AML and AML LSCs (CD45^{dim} CD34⁺ CD38⁻) in n=5 representative AML patient samples (see Table 1). Histograms show staining with anti-siglec-6 mAb (grey) and isotype control antibody (white histograms). Inset numbers state the normalized mean fluorescence intensity (NMFI) obtained by staining with anti-siglec-6 mAb and isotype control. The plots show cytolytic activity of CD8⁺ JML-1_28z CAR, JML-1_BBz CAR, FLT3_28z CAR, and untransduced (UTD) T-cells against LSC and bulk AML blasts in a flow cytometry-based assay (24-hour co-culture). The experiment was performed in triplicate wells with 10,000 target cells/well. Counting beads were used to quantitate the number of residual live target cells at the end of co-culture. (B) Correlation between tumor specific cell killing by CD8⁺ JML-1_BBz CAR T-cells (flow cytometry-based assay, 24-hour co-culture; 2.5:1 E:T ratio) and siglec-6 NMFI expression on primary AML cells. (C) Siglec-6 expression on bulk AML and AML LSC in n=10 AML patients. The patients were ranked in ascending order of NMFI (see Table 1).

FIG. 3. JML-1-CAR T-cells confer potent anti-leukemia activity in a xenograft model of AML in vivo. Female NSG mice were inoculated with 2×10⁶ U937 AML cells (ffluc⁺ GFP⁺) and on day 6 were treated with 5×10⁶ CAR-modified or untransduced (UTD) T-cells. T-cells were formulated in CD4*:CD8⁺=1:1 ratio. (A) Serial bioluminescence (BL) imaging to assess leukemia progression and/or regression. Note the scale indicating upper and lower BL thresholds at each analysis time point (right). (B) Flow cytometric analysis of PB on day 10, 14 and day 45 to detect T-cells and leukemia cells. Human T-cells in mice PB were defined as 7-AAD-CD45⁺CD3⁺ cells. Leukemia cells were defined as 7-AAD-CD45+GFP+ cells. ****p<0.0001 (Student's t test) (C) Waterfall plot showing change in absolute BL values between day 6 and day 10 after tumor inoculation. BL values were obtained as photon/sec/cm2/sr in regions of interest encompassing the entire body of each mouse. (D) Percentage of leukemic cells detected in BM, spleen and PB by flow cytometry at the end of experiment. ****p<0.0001 **p<0.05 *p<0.5 (Student's t test). (E-F) Kaplan-Meier survival analysis (E) overall survival and (F) progression free survival from different treatment groups. Data shown are representative for results obtained in independent experiments with JML-1-CAR T-cell from n=2 donors. ****p<0.0001, Log-rank (Mantel-Cox) test. (G-H) Female NSG mice were inoculated with 1×10⁶ MOLM-13 AML cells (ffluc+ GFP+) and on day 4 and 7 were treated with 5×10⁶ CAR-modified or untransduced (UTD) T-cells. T-cells were formulated in CD4*:CD8*=1:1 ratio. (G) Waterfall plots showing change in absolute BL values between day 7 and day 10 after tumor inoculation. (H) Kaplan-Meier survival analysis from each treatment group. ****p<0.0001, Log-rank (Mantel-Cox) test.

FIG. 4. Human HSC/P do not express siglec-6 and are preserved after in vitro co-culture with JML-1-CAR T-cells. (A) Flow cytometric analysis of siglec-6-expression on G-CSF-mobilized CD34*CD38—HSCs and CD34⁺CD38+progenitors from PB of n=5 HDs. Inset values state the NMFI. NMFI is calculated by dividing MFI of anti-siglec-6 mAb (grey) with MFI of isotype control (white histograms). (B) Right panel: Percentage of live (7-AAD-) HSCs after 24-hour co-incubation with CD8+JML-1_BBz CAR, CD123 CAR or untransduced T-cells. Assay was performed in triplicate wells with 5,000 target cells/well. Counting beads were used to quantitate the number of residual live HSCs at the end of co-culture. Data from n=3 independent experiments are shown. Left panel: Colony formation assay performed with residual live HSCs after 24 hours of co-incubation with CD8+JML-1_BBz CAR, CD123 CAR or untransduced T-cells. Diagram shows the absolute number of colonies (mean±s.d.) per 55 mm plate as determined by microscopy on day 14 from n=3 independent experiments. GEMM (Granulocyte/Erythroid/Macrophage/Megakaryocyte); GM (Granulocyte/Macrophage); CFU-E (Colony Forming Unit-Erythroid); CFU-M (Colony Forming Unit-Macrophage); CFU-G (Colony forming unit-Granulocyte). (C) Flow cytometric analysis of cell surface expression of different CAR-target antigens on CD34⁺ and CD34⁺CD38⁺ cells from HD (n=5). Values state the normalized mean fluorescence intensity. (D) ****p<0.0001 **p <0.05 *p<0.5 (Student's t test).

FIG. 5. Siglec-6 is expressed on malignant B-lymphocytes in B-CLL, and on healthy memory B-cells. (A) Flow cytometric analysis of siglec-6-expression on CLL cells from n=10 patients. Patients' characteristics are summarized in Table-2. Histograms show staining with anti-siglec-6 mAb (grey) and isotype control antibody (white histograms). Inset numbers state the NMFI. (B) Specific cytolytic activity of CD8⁺ JML-1_28z CAR, JML-1_BBz CAR, CD19_BBz CAR, and untransduced (UTD) T-cells against CLL cells in a flow cytometry-based assay. Target cells were seeded in triplicate wells (10,000 cells/well) and co-cultured with effector cells at E:T ratio 5:1. Counting beads were used to quantitate the number of residual live target cells after 4-hour of co-culture. (C) Correlation between CLL specific cell killing by CD8⁺ JML-1_BBz or JML-1_28z CAR T-cells (after 4-hour co-culture, 5:1 E:T ratio) and siglec-6 normalized expression on primary B-cell cells. Simple linear correlation was calculated (R squared=0.54; p=0.01 and R squared 0.29; p=0.1 for JML-1_BBz CAR and JML-1_28z CAR, respectively). (D) Flow cytometric analysis of siglec-6-expression on healthy B-cells (CD45⁺CD19⁺CD5-CD20^{high}) from B-CLL patients. Left: pooled data of siglec-6-expression on B-CLL cells from n=10 patients and on healthy B-cell subsets from 5 out of 10 B-CLL patients. The remaining n=5 patients did not have enough healthy B-cells in the PB for subset analysis. Right: A representative histogram from patient-3, which shows siglec-6-expression on healthy immature (CD45⁺CD19⁺CD5-CD20^{high}CD10⁺), naïve (CD45⁺CD19⁺CD20^{high} CD5-CD10-CD27⁻) and memory (CD45⁺CD19⁺CD5-CD20^{high}CD10-CD27⁺) B-cells compared to B-CLL cells. (E) Flow cytometric analysis of siglec-6-expression on healthy PBMCs from n=7 HD. Siglec-6-expression by B-cells (CD45⁺CD19⁺), Myeloid cells (CD45⁺CD33⁺), T cells (CD45⁺CD3⁺CD56⁻), NK cells (CD45⁺CD56⁺CD3⁻), NKT cells (CD45⁺CD3⁺CD56⁺) in n=7 HD. Siglec-6-expression by siglec-6-positive (U937, TF-1, MV4;11 and MOLM-13) negative (K562, JeKo-1) cell lines are plotted for reference. Graph and histograms in right show siglec-6-expression on memory or naïve and immature cells form n=5 HD (left histogram: memory B-cells, right histogram; naïve/immature B-cells). (F) Siglec-6-expression on healthy B-cells from CLL patients and HD. *p<0.5, **p<0.05 (Student's t test).

FIG. 6. Design of CAR constructs, CAR expression and phenotype of CD4⁺ and CD8⁺ T-cells. (A) Design of CAR used in the study. Single chain variable fragments (scFv; V_{H}-Linker-V_{L}) were derived from mAbs JML-1 (siglec-6 specific CAR), 4G8 (FLT3-specific CAR), FMC63 (CD19-specific CAR), and 32716 (CD123-specific CAR). The scFvs were fused to an IgG4 hinge spacer and CD28 transmembrane domain to the intracellular signaling module. CD28 or 4-1BB and CD3z were incorporated as costimulatory and signaling domains, respectively. A truncated epidermal growth factor receptor (EGFRt) (separated from CAR transgene by T2A ribosomal skip sequence) was incorporated for detection and enrichment of CAR-positive T-cells. (B) Dot plots show expression of the EGFRt marker on CD4⁺ and CD8⁺ T-cells after transduction. (C) Purity of CAR-positive T-cell after enrichment of EGFRt⁺ CD8⁺ and CD4⁺ T-cells prior to functional testing. Untransduced T-cells are included for comparison in B-C. (D) Summary data of percentage of CAR-positive T-cells from HD after enrichment.

FIG. 7. Specificity and selectivity of JML-1-CAR T-cells for Siglec-6-expressing target cells. (A) Flow cytometric analysis of siglec-6 expression by native K562 and K562/siglec-6 cells. Histograms show staining with anti-siglec-6 mAb (grey) and isotype control antibody (white histograms). Inset numbers state the NMFI. (B) Left panel: Specific cytolytic activity of CD8+JML-1_28z CAR, JML-1_BBz CAR, FLT3_28z CAR and untransduced (UTD) T-cells against K562/siglec-6 cells, analysed in a bioluminescence-based assay after 4 hour co-culture. Right panel: Summarized data of cytotoxicity assay (co-culture of 24 hours, 10:1 E:T ratio) of CAR T-cells from n=3 HDs. Values are presented as mean±s.d. (C) ELISA to detect IFN-γ and IL-2 in supernatant after 24-hour co-cultures. T-cells and target cell were seeded in triplicate wells at 2:1 E:T ratio. Values are presented as mean±s.d. (D) Summary data of cytokine production (IFN-γ and IL-2) by CD4⁺ T-cells from n=3 different donors. (E) Proliferation of T-cells after 72 h co-culture analysed by CFSE dye dilution. Assay was performed in triplicate wells at 2:1 E:T ratio. Histograms show proliferation of live (7-AAD-) T-cells. No exogenous cytokines were added to the assay medium. ***p<0.001, **** p<0.0001 (Student's t test).

FIG. 8. Recognition of TF-1 and Kasumi-1 tumor cell lines by JML-1-CAR T-cells. (A) Flow cytometric analysis of siglec-6 expression on TF-1 (erythroleukemia) and Kasumi-1 (AML with t(8;21)). Histograms show staining with anti-siglec-6 mAb (grey) and isotype control antibody (white histograms). Inset numbers state the NMFI. (B) Specific cytolytic activity of CD8⁺ JML-1_28z CAR, JML-1_BBz CAR, FLT3_28z CAR and untransduced (UTD) T-cells against TF-1 and Kasumi-1 after 4-hour co-culture, analyzed in a luminescence-based assay. TF-1 cell line does not express FLT3 while Kasumi-1 expresses low level of FLT3. (C) ELISA to detect IFN-γ and IL-2 in supernatant after 24-hour co-cultures. T-cells and target cell were seeded in triplicate wells at 2:1 E:T ratio. Values are expressed as mean±s.d. (D) Proliferation of CD4⁺ T-cells after 72-hour co-culture analysed by CFSE dye dilution. T-cells and target cell were seeded in triplicate wells at 2:1 E:T ratio. Proliferation of live (7-AAD) T-cells is shown in histograms. No exogenous cytokines were added to the assay medium.

FIG. 9. Leukemia stem cells (LSC) in primary AML samples. (A) Flow cytometry-gating strategy of primary AML blasts. Siglec-6 expression is analyzed on live (7-AAD-) bulk AML cells (CD45aim) and on AML LSCs (CD45^{dim}CD34⁺CD38⁻). (B) Siglec-6 expression on AML blasts with phenotypic heterogeneity. Histograms show staining with anti-siglec-6 mAb (grey) and isotype control antibody (white histograms). Inset numbers state the NMFI.

FIG. 10. Generation and functional analysis of JML-1-CAR T-cells that were derived from AML patients. (A) CAR transduction efficiency in CD4⁺ and CD8⁺ T-cells shown as EGFRt expression before and after CAR-positive T-cell enrichment. (B) CD4⁺ and CD8⁺ T-cell expansion after CAR transduction. (C) Cytolytic activity of CD8⁺ JML-1_BBz CAR, FLT3_28z CAR, CD123_28z CAR and untransduced (UTD) T-cells against AML cell lines. (D) IFN-γ and IL-2 production (ELISA) after in 24-hour co-culture of CD4⁺ T-cells with AML cell lines. T-cells and target cell were seeded in triplicate wells at 2:1 E:T ratio. Values are presented as mean±s.d. (E) Proliferation of CD4⁺ T-cells after 72-hour co-culture analyzed by CFSE dilution. T-cells and target cell were seeded in triplicate wells at 2:1 E:T ratio. Proliferation of live T-cells is shown in histograms. No exogenous cytokines were added to the assay medium. Data shown in B-E correspond to CAR T-cells from one representative patient of at least n=3 AML patients.

FIG. 11. Anti-leukemia activity of patient-derived JML-1-CAR T-cells against autologous AML blasts. (A) Specific cell lysis by CD8⁺ JML-1_28z CAR, JML-1_BBz CAR, FLT3_28z CAR and untransduced (UTD) T-cells against autologous ‘bulk’ AML blasts, AML LSCs and U937 cells. (B) IFN-γ production (ELISA) after in 24-hour co-culture of CD4⁺ T-cells with autologous AML blasts and U937 cells. T-cells and target cell were seeded in triplicate wells at 2:1 E:T ratio. Values are presented as mean±s.d. (C) Proliferation of CD4⁺ T-cells after 72-hour co-culture analyzed by CFSE dilution. T-cells and target cell were seeded in triplicate wells at 2:1 E:T ratio. Proliferation of live (7-AAD-) T-cells is shown in histograms. No exogenous cytokines were added to the assay medium. Data shown correspond to CAR T-cells from one representative patient of at least n=3 patients.

FIG. 12. Leukemia burden and CAR T-cell persistence in BM of mice in the xenograft model of AML. (A) Dot plots show the frequency of T-cells (CD45⁺CD3⁺) and leukemic cells (CD45*GFP+) in BM, as percentage of live (7-AAD-) cells in one representative mouse per group.

FIG. 13. Expression of candidate target antigens for CAR T-cells in AML on normal HSC/P. (A) Gating strategy to identify HSC (CD34⁺) and HPC (CD34⁺CD38⁻) cells from G-CSF mobilized PB cells from HD. (B) Expression of different potential CAR antigens on HSC (upper panel) and HPC (lower panel). Histograms show expression of antigen (grey) against staining with isotype control mAb (white histograms). Inset numbers show the NMFI. Data are representative from n=5 HDs.

FIG. 14. Malignant and normal B-cells in patients with B-CLL and in HD. (A) Flow cytometry-gating strategy of primary CLL cells. Siglec-6 expression is analyzed on live (7-AAD-) healthy B-cells (CD45⁺CD19⁺CD20^{high} CD5⁻) and B-CLL cells (CD45⁺CD19⁺CD20^{mid/low}CD5⁺) (B and C) Immature cells are CD45^{high}CD19⁺CD10⁺CD5⁻, naïve B-cells: CD45^{high}CD19⁺CD5⁻CD27⁻CD38⁻ and memory B-cells: CD45^{high}CD19⁺CD5⁻CD27+. Histograms show staining with anti-siglec-6 mAb (grey) and isotype control antibody (white histograms). Inset numbers state the NMFI.

FIG. 15. Recognition of normal B-cells from B-CLL patients by JML-1-CAR T-cells. (A) Specific cytolytic activity of CD8⁺ JML-1_28z CAR, JML-1_BBz CAR, CD19_BBz CAR, and untransduced (UTD) T-cells against healthy CD19+B-cells in a flow cytometry-based assay. Counting beads were used to quantitate the number of residual live target cells after 4-hour of co-culture.

### DETAILED DESCRIPTION OF INVENTION

Unless specifically defined herein, all technical and scientific terms used herein have the same meaning as commonly understood by a skilled artisan in the fields of cancer immunotherapy, gene therapy, immunology, biochemistry, genetics, and molecular biology.

All methods and materials similar or equivalent to those described herein can be used in the practice or testing of the present invention, with suitable methods and materials being described herein.

The term “about” used in the context of the present invention means that the value following the term “about” may vary within the range of +/−20%, preferably in the range of +/−15%, more preferably in the range of +/−10%.

All publications, patents and patent applications cited herein are hereby incorporated by reference in their entirety for all purposes. References referred to herein are indicated by a reference number in square brackets (e.g. as “[31]” or as “reference [31]”), which refers to the respective reference in the list of references at the end of the description. In case of conflict, the present specification, including definitions, will prevail over the cited references. Further, the materials, methods, and examples are illustrative only and are not intended to be limiting, unless otherwise specified.

As used herein, each occurrence of terms such as “comprising” or “comprises” may optionally be substituted with “consisting of” or “consists of”.

Siglec-6-Binding Polypeptide

The present invention relates to a siglec-6-binding polypeptide that comprises or consists of an antibody or a fragment thereof binding siglec-6 or a chimeric antigen receptor (CAR), preferably a CAR.

Specifically provided is a siglec-6-binding polypeptide that comprises or consists of an antibody or a fragment thereof binding to siglec-6.

The term “antibody or a fragment thereof” includes, for example, monoclonal, chimeric, single chain, humanized and human antibodies. It also includes, for example, Fab fragments, F(ab′)₂, Fv, scFv fragments or single domain antibodies such as domain antibodies or nanobodies, single variable domain antibodies or immunoglobulin single variable domain comprising merely one variable domain, which might be VHH, VH or VL, that specifically bind an antigen or epitope independently of other variable regions or domains. Said term also includes diabodies or Dual-Affinity ReTargeting (DART) antibodies. Further envisaged are (bispecific) single chain diabody, tandem diabody (Tandab), bispecific T cell engager (BiTE) antibodies and tri-specific T cell engaging antibodies such as hemibodies. Any such antibodies and fragments thereof, as well as their production is commonly known in the art.

Preferably, the polypeptide comprising or consisting of antibody or the fragment thereof binding siglec-6 is at least bispecific. However, it can also be multispecific, such as trispecific or tetra specific. Bispecific, trispecific, etc. means that that the polypeptide is able to bind to two, three, etc. different target antigens simultaneously or sequentially.

Thus, the siglec-6-binding polypeptide can comprise or consist of a first antibody or a fragment thereof binding to siglec-6 and a second antibody or fragment thereof binding to a target other than siglec-6, that can optionally be connected to each other via a linker.

The antibody or a fragment thereof binding to siglec-6 can for example be represented by an amino acid sequence shown in SEQ ID NO: 25 or by an amino acid sequence having at least 90%, preferably 95%, more preferably 97% or most preferably 99%, sequence identity with the amino acid sequence shown in SEQ ID NO: 25 and has siglec-6-binding ability. Preferably, the antibody or fragment thereof binding to siglec-6 is represented by an amino acid sequence shown in SEQ ID NO: 25.

The at least bispecific siglec-6-binding polypeptide is preferably capable of binding to an immune cell, such as a T cell or an NK cell, preferably to a T cell. However, binding to other immune cells, such as macrophages, is also encompassed.

Accordingly, the at least bispecific siglec-6-binding polypeptide preferably additionally binds to (human) CD3, such as CD3 epsilon or CD3zeta, preferably CD3zeta. CD3 is expressed on T cells and forms part of the T cell receptor. Thus, the bispecific polypeptide can recruit effector cells, such as T cells or NK cells, to target cells expressing siglec-6 on their surface, by binding simultaneously to siglec-6 and to e.g. CD3. Antibodies to human CD3 are well known in the art, see for example the antibodies against N-terminal amino acids 1-27 of CD3 epsilon in WO 2008/119567, incorporated herein by reference.

Thus, the at least bispecific siglec-6-binding polypeptide is preferably capable of recruiting an immune cell, such as a T cell, an NK cell, preferably a T cell, to a target cell expressing siglec-6 on its surface.

In another embodiment, the siglec-6-binding polypeptide is conjugated to another compound, such as a detectable marker or a drug. It is preferred in this embodiment that the polypeptide is conjugated to a drug. The drug can, for example, be a toxin. The toxin is preferably capable of killing target cells expressing siglec-6 on their surface. Examples of such toxins include Maytansin, Auristatin, Taxoid and PNU anthracycline.

In a preferred embodiment, the siglec-6-binding polypeptide is a chimeric antigen receptor (CAR). A CAR is a receptor that can be expressed on the surface of a cell and that can bind to a ligand, e.g. expressed on the surface of another cell. The receptor can thereby lead to recruitment of a cell expressing the receptor to target cells that express the ligand on their surface. Moreover, upon binding to the ligand the CAR can optionally transmit an intracellular signal within the cells on which it is expressed. Thus, for example the CAR can be expressed on a T cell, and upon binding to its ligand activate the T cell.

In a more specific embodiment, the CAR thus comprises at least one extracellular ligand binding domain, a transmembrane domain and at least one intracellular signalling domain, wherein said extracellular ligand binding domain preferably comprises a siglec-6-binding element. The extracellular domain can further comprise a spacer domain, such as a spacer domain from CD8a, IgG3 or IgG4. The transmembrane domain can comprise a CD28 transmembrane domain. The intracellular signalling domain can comprise a costimulatory domain and a CD3 zeta (CD3i) domain.

In an embodiment of the invention, the siglec-6-binding element is represented by an amino acid sequence having at least 90%, preferably 95%, more preferably 97% or most preferably 99% sequence identity with an amino acid sequence shown in SEQ ID NO: 25 and has siglec-6-binding ability. Preferably, the siglec-6-binding element is represented by an amino acid sequence shown in SEQ ID NO: 25.

In an embodiment of the invention, the spacer domain is represented by an amino acid sequence having at least 90%, preferably 95%, more preferably 97% or most preferably 99% sequence identity with an amino acid sequence shown in SEQ ID NO: 9 or 11. Preferably, the spacer domain is represented by an amino acid sequence shown in SEQ ID NO: 9 or 11. The spacer connects the extracellular targeting and the transmembrane domain. It affects the flexibility of the siglec-6-binding element, reduces the spatial constraints from CAR to ligand and therefore impacts epitope binding. Binding to epitopes with a membrane-distal position often require CARs with shorter spacer domains, binding to epitopes which lie proximal to the cell surface often require CARs with long spacer.

In an embodiment of the invention, the transmembrane domain is represented by an amino acid sequence having at least 90%, preferably 95%, more preferably 97% or most preferably 99% sequence identity with an amino acid sequence shown in SEQ ID NO: 13. Preferably, the transmembrane domain is represented by an amino acid sequence shown in SEQ ID NO: 13.

The CD28 transmembrane domain consists of a hydrophobic alpha helix, traverses the membrane of the cell and anchors the CAR to the cell surface. It impacts the expression of the CAR on the cell surface.

In an embodiment of the invention, the costimulatory domain of the siglec-6-CAR polypeptide is a CD28 cytoplasmic domain or a 4-1BB costimulatory domain.

In an embodiment of the invention, the intracellular signalling domain comprises a CD28 cytoplasmic domain and a CD3 zeta domain. In another embodiment of the invention, the intracellular signalling domain comprises a 4-1BB costimulatory domain and a CD3 zeta domain.

In an embodiment of the invention, the CD28 cytoplasmic domain is represented by an amino acid sequence having at least 90%, preferably 95%, more preferably 97% or most preferably 99% sequence identity with an amino acid sequence shown in SEQ ID NO: 15. Preferably, the CD28 cytoplasmic domain is represented by an amino acid sequence shown in SEQ ID NO: 15. The CD28 cytoplasmic domain is a costimulatory domain and is derived from intracellular signaling domains of costimulatory molecules.

In an embodiment of the invention, the 4-1BB costimulatory domain is represented by an amino acid sequence having at least 90%, preferably 95%, more preferably 97% or most preferably 99% sequence identity with an amino acid sequence shown in SEQ ID NO: 17. Preferably, the 4-1BB costimulatory domain is represented by an amino acid sequence shown in SEQ ID NO: 17.

In an embodiment of the invention, the CD3 zeta domain is represented by an amino acid sequence having at least 90%, preferably 95%, more preferably 97% or most preferably 99% sequence identity with an amino acid sequence shown in SEQ ID NO: 19. Preferably, the CD3 zeta domain is represented by an amino acid sequence shown in SEQ ID NO: 19. The CD3 zeta domain mediates downstream signaling during the T cell activation. It is derived from the intracellular signaling domain of the T cell receptor and contains ITAMs (immunoreceptor tyrosine based activation motifs).

In an embodiment of the invention, the extracellular domain comprises an amino acid sequence having at least 90%, preferably 95%, more preferably 97% or most preferably 99% sequence identity to an amino acid sequence shown in SEQ ID NO: 35 or 37. Preferably, the extracellular domain comprises an amino acid sequence shown in SEQ ID NO: 35 or 37. More preferably, the extracellular domain consists of an amino acid sequence shown in SEQ ID NO: 35 or 37.

In an embodiment of the invention, the intracellular signalling domain comprises an amino acid sequence having at least 90%, preferably 95%, more preferably 97% or most preferably 99% sequence identity to an amino acid sequence shown in SEQ ID NO: 39 or 41. Preferably, the intracellular signalling domain comprises an amino acid sequence shown in SEQ ID NO: 39 or 41. More preferably, the intracellular signalling domain consists of an amino acid sequence shown in SEQ ID NO: 39 or 41.

Thus, said extracellular domain can comprise an amino acid sequence shown in SEQ ID NO: 35 or 37 or an amino acid sequence having at least 90% identity to an amino acid sequence shown in SEQ ID NO: 35 or 37, said transmembrane domain can comprise an amino acid sequence shown in SEQ ID NO: 13 or an amino acid sequence having at least 90% identity to an amino acid sequence shown in SEQ ID NO: 13 and said intracellular signalling domain can comprise an amino acid sequence shown in SEQ ID NO: 39 or 41 or an amino acid sequence having at least 90% identity to an amino acid sequence shown in SEQ ID NO: 39 or 41.

In a preferred embodiment of the invention, the siglec-6-CAR polypeptide comprises an amino acid sequence having at least 90%, preferably 95%, more preferably 97% or most preferably 99% sequence identity to an amino acid sequence shown in any one of SEQ ID NOs: 27, 29, 31 or 33. Preferably, the siglec-6-CAR polypeptide comprises an amino acid sequence shown in any one of SEQ ID NOs: 27, 29, 31 or 33. More preferably, the siglec-6-CAR polypeptide consists of an amino acid sequence shown in any one of SEQ ID NOs: 27, 29, 31 or 33.

In embodiments that relate to a siglec-6-binding CAR comprising a variant element defined by % sequence identity with a specific SEQ ID NO, the CAR ideally retains its ability to function as siglec-6-binding CAR (including e.g. ligand binding and/or lymphocyte activation), and the ability to function as siglec-6-binding CAR is ideally at least the same as for a CAR of the same sequence but in which the element in question is represented by the SEQ ID NO without variation. For example, if a CAR comprises a spacer domain having at least 90% sequence identity with SEQ ID NO: 11, the CAR ideally has the same ability to function as siglec-6-binding CAR as a CAR of the same sequence except for a spacer represented by SEQ ID NO: 11 (i.e. without variation).

A CAR polypeptide can also be specific to more than one target. Thus, the invention also provides a siglec-6-binding polypeptide comprising or consisting of a CAR that comprises at least two binding elements at least one of which binds to siglec-6 and/or that comprises at least one binding element that is a switchable/programmable binding domain, that can be switched/programmed to bind to siglec-6.

Polynucleotide Encoding the Siglec-6-Binding Polypeptide

The present invention relates to a polynucleotide or set of polynucleotides encoding the siglec-6-binding polypeptide of the present invention as defined above.

In an embodiment of the present invention, the polynucleotide encoding the polypeptide of the present invention is further flanked by a left and a right inverted repeat/direct repeat (IR/DR) segments. The flanking segment in 5′-direction is represented by a left inverted repeat/direct repeat (IR/DR) segment and the flanking segment in 3′-direction is represented by a right inverted repeat/direct repeat (IR/DR) segment.

The nucleotide sequences of the left IR/DR segment and the nucleotide sequences of right IR/DR segment may be recognized by a transposase protein. The transposase is not particularly limited and can be, for example, Sleeping Beauty transposase or PiggyBac transposase.

Preferably, the left IR/DR segment comprises a nucleotide sequence having at least 90%, preferably 95%, more preferably 97% or most preferably 99% or even 100% sequence identity to the nucleotide sequence shown in SEQ ID NO: 43. Similarly, the right IR/DR segment comprises a nucleotide sequence having at least 90%, preferably 95%, more preferably 97% or most preferably 99% or even 100% sequence identity to the nucleotide sequence shown in SEQ ID NO: 44.

The term “is flanked by” indicates that further nucleotides are present in the 5′-region and in the 3′-region of the polynucleotide sequence encoding the polypeptide which are all located on the same polynucleotide. Hence, the polynucleotide sequence encoding the polypeptide is flanked by IR/DR sequences, i.e. flanking segments, such that the presence of a transposase allows the integration of the polynucleotide encoding the polypeptide as well as the nucleotide sequences corresponding to the flanking segments into the genome of the transfected cell. In an aspect, the polynucleotide which is integrated into the genome comprises a polynucleotide encoding the polypeptide and an optional detectable marker gene such as an EGFRt marker and is flanked by flanking segments. In this aspect, the region of the nucleotide sequence corresponding to the coding regions of the polypeptide and the EGFRt marker is considered to represent the reference segment.

When used in the present invention, the term “is flanked by” also means that the distance between a flanking segment and a reference segment to be less than 1000 bp, 900 bp, 800 bp, 700 bp, 600 bp, 500 bp, 400, 300 bp, 200 bp, 100 bp, 50 bp, 20 bp or less than 10 bp.

In this respect, the reference segment is the region corresponding to the coding region of the polynucleotides which are integrated into the genome. The overall architecture of the polynucleotide which is integrated into the genome of the transfected cell may be as follows (5′ to 3′ direction): [left IR/DR sequence]—[reference segment]—[right IR/DR sequence].

The distance between a flanking segment and a reference segment may be determined by counting the nucleotides between the 3′-end of the left IR/DR sequence and the 5′-end of the reference segment. Similarly, the distance between a flanking segment and a reference segment may be determined by counting the distance between the 3′-end of the reference segment and the 5′-end of the right IR/DR sequence. Both distances may be in the same such that the reference segment is centred between the flanking segments or the distances may be different.

The distance between the 3′-end of the left IR/DR sequence and the 5′-end of the reference segment may be less than 1000 bp, 900 bp, 800 bp, 700 bp, 600 bp, 500 bp, 400 bp, 300 bp, 200 bp or less than 100 bp.

The distance between the 3′-end of the reference segment and the 5′-end of the right IR/DR sequence may be less than 200 bp, 100 bp, 50 bp, 20 bp or less than 10 bp.

In an exemplary embodiment of the invention, the distance between the 3′-end of the left IR/DR sequence and the 5′-end of the reference segment may be less than 700 bp and the distance between the 3′-end of the reference segment and the 5′-end of the right IR/DR sequence may be less than 10 bp.

In an exemplary embodiment of the invention, the distance between the 3′-end of the left IR/DR sequence and the 5′-end of the reference segment may be less than 700 bp and more than 600 bp and the distance between the 3′-end of the reference segment and the 5′-end of the right IR/DR sequence may be less than 10 bp and more than 5 bp.

In an aspect, the polynucleotide which is integrated into the genome comprises a polynucleotide encoding the siglec-6-binding polypeptide and a detectable marker gene such as an EGFRt marker and is flanked by flanking segments. In this aspect, the region of the nucleotide sequence corresponding to the coding regions of the siglec-6-binding polypeptide and the EGFRt marker is considered to represent the reference segment.

In an embodiment, the polynucleotide sequence of the invention comprises a sequence represented by SEQ ID NO: 26 or a nucleotide sequence having at least 80% sequence identity, such as at least 90%, preferably at least 95% sequence identity to nucleotide sequence shown in SEQ ID NO: 26. The polynucleotide sequence of the invention preferably comprises a nucleotide sequence represented by SEQ ID NO: 26.

Thus, in a related embodiment of the present invention, the polynucleotide of the invention relates to a polynucleotide sequence comprising a sequence having at least 90%, preferably 95%, more preferably 97% or most preferably 99% or even 100% sequence identity to a nucleotide sequence shown in any one of SEQ ID NO: 28, 30, 32 or 34. Preferably, the polynucleotide of the invention comprises a nucleotide sequence shown in any one of SEQ ID NO: 28, 30, 32 or 34. The polynucleotide of the invention can also consist of a nucleotide sequence shown in any one of SEQ ID NO: 28, 30, 32 or 34.

Expression Vector

The present invention relates to an expression vector comprising a polynucleotide or set of polynucleotides of the present invention as defined herein.

A wide range of expression vectors for polypeptides are known in the art and are further detailed herein. For example, in some embodiments of the invention, the expression vector is a non-viral or viral vector, and—in the context of medical purposes—preferably a non-viral vector.

The expression vector can be a minimal DNA expression cassette. Moreover, an expression vector may be a DNA expression vector such as a plasmid, linear expression vector or an episome. In certain aspects, the vector comprises additional sequences, such as sequences that facilitate expression of the polypeptide, such as a promoter, enhancer, poly-A signal, and/or one or more introns. In certain aspects, the expression vector may be a transposon donor DNA molecule, preferably a minicircle DNA.

The present invention also relates to minicircle DNA comprising a polynucleotide of the present invention as defined herein. As used herein, the term “minicircle DNA” refers to vectors which are supercoiled DNA molecules that lack a bacterial origin of replication and an antibiotic resistance gene. Therefore, they are primarily composed of a eukaryotic expression cassette.

In a useful embodiment the minicircle DNA of the invention is introduced into the cell in combination with the transposase protein or a nucleic acid (e.g. DNA or mRNA) encoding a transposase protein (such as Sleeping Beauty or PiggyBac) by electrotransfer, such as electroporation, nucleofection; chemotransfer with substances such as lipofectamin, fugene, calcium phosphate; nanoparticles, or any other conceivable method suitable to transfer material into a cell.

A viral vector can be, for example, a gamma retroviral vector or a lentiviral vector. Such vectors and their construction and production are commonly known in the art.

The polynucleotide or expression vector can be introduced into immune cells by any suitable means, such as by transfection or by transduction. Transfection refers to chemical or physical delivery into the cells, e.g. by electrotransfer, such as electroporation, nucleofection; chemotransfer with substances such as lipofectamin, fugene, calcium phosphate, PEI. Transduction refers to other means of (targeted) delivery into the cells including delivery by a viral vector or nanoparticles. However, the present invention is not limited to any particular method of delivery of genetic material into immune cells, such that also any other conceivable method suitable to transfer genetic material into a cell can be used in the context of the invention.

Immune Cell

The present invention also relates to an immune cell (preferably a lymphocyte, more preferably a T cell) comprising a polypeptide and/or a polynucleotide or set of polynucleotides and/or an expression vector of the present invention as defined herein.

The present invention also relates to an immune cell (preferably a lymphocyte, more preferably a T cell) comprising a polypeptide of the present invention as defined herein.

The present invention also relates to an immune cell (preferably a lymphocyte, more preferably a T cell) comprising a polynucleotide or set of polynucleotides of the present invention as defined herein.

The present invention also relates to an immune cell (preferably a lymphocyte, more preferably a T cell) comprising an expression vector of the present invention as defined herein.

The immune cell can be a recombinant immune cell. A “recombinant immune cell” refers to an immune cell that has been modified to comprise a molecule, such as a polypeptide or a polynucleotide, in particular a polynucleotide, that is not comprised in the same cell without said modification, e.g. a native immune cell obtained from a (human) subject.

The immune cell (preferably lymphocyte, more preferably T cell) is preferably also capable of expressing the polynucleotide of the present invention. Thereby, the siglec-6-binding polypeptide which is encoded by the polynucleotide of the invention is translated and integrated into the cell membrane of the immune cell.

Expression of the siglec-6-binding polypeptide allows the immune cell (preferably lymphocyte, more preferably T cell) of the present invention to acquire specific reactivity against target cells expressing the siglec-6 antigen, including leukemia cells. Such immune cells, e.g. siglec-6 CAR-T cells, are able to recognize and (antigen-specifically) eradicate leukemia cells, and more specifically AML, CLL, MALT lymphoma, clonal mast cell disease cells or thymoma. Such cells are able to proliferate and to induce an immune response after encountering the siglec-6 antigen.

In some useful applications, the immune cell can also be modified to bind to at least one additional target apart from siglec-6. Thus, the invention also provides an immune cell that comprises one, two or more CAR constructs that each targets a distinct target antigen, at least one of them being siglec-6, and/or a polynucleotide or set of polynucleotides encoding such CARs and/or an expression vector comprising a polynucleotide or set of polynucleotides encoding such CARs.

The invention also provides an immune cell that comprises a single CAR construct that comprises one, two or more binding elements, at least one of them binding siglec-6, and/or a polynucleotide or set of polynucleotides encoding such a CAR and/or an expression vector comprising a polynucleotide or set of polynucleotides encoding such a CAR.

The invention also provides an immune cell that comprises a CAR where at least one binding domain is a switchable/programmable binding domain, that can be switched/programmed to be bind to siglec-6, and/or a polynucleotide or set of polynucleotides encoding such a CAR and/or an expression vector comprising a polynucleotide or set of polynucleotides encoding such a CAR.

The immune cell is preferably a lymphocyte, such as a T cell or an NK cell. However, other immune cells, such as macrophages, are also encompassed. AT cell is especially preferred.

In an embodiment of the present invention, the immune cell (preferably lymphocyte, more preferably T cell) is a CD4⁺ T cell or a CD8⁺ T cell.

In an embodiment of the present invention, the immune cell (preferably lymphocyte, more preferably T cell) is a CD4⁺ T cell.

In an embodiment of the present invention, the immune cell (preferably lymphocyte, more preferably T cell) is a CD8⁺ T cell.

In an embodiment of the invention, the immune cell of the present invention may further express a marker gene, e.g. the EGFRt marker on the cell surface. The EGFRt marker can be used to detect, track, select and deplete the immune cell of the present invention.

Therefore, analysis of drug product persistence following administration of the immune cell is made available. Furthermore, the EGFRt marker makes immune cells of the invention sensitive to ADCC/CDC through the antibody Cetuximab which can therefore be used as safety switch.

The amino acid sequence of the EGFRt which may be used in the present invention is represented by SEQ ID NO: 23.

In an embodiment of the invention, the immune cell is (or has been) obtained from an immune cell (preferably lymphocyte, more preferably T cell) derived from a mammal, preferably a human. Preferably, the immune cell is (or has been) obtained from a subject that is to be treated with the immune cell after it has been modified to comprise the siglec-6-binding polypeptide, e.g. by a method as described herein. Alternatively, the immune cell that has been modified to comprise the siglec-6-binding polypeptide is (or has been) obtained from a healthy (allogeneic) donor, an (autologous or allogeneic) cord blood unit, or an induced pluripotent stem cell.

Method for Producing Immune Cells

The present invention also relates to a method for producing immune cells (preferably lymphocyte, more preferably T cell) of the present invention as defined herein.

In an embodiment of the present invention, the method for producing immune cells comprises the steps of (a) isolating immune cells from a (peripheral) blood sample of a subject, (b) transforming or transducing the immune cells with a polynucleotide or expression vector as described above, optionally followed by (c) purifying the transfected or transduced immune cells. The method may further comprise formulating the immune cells into a formulation that is suitable for administration to a human subject.

Preferably, in step (b), the immune cells are transformed using a transposable element comprising a polynucleotide or set of polynucleotides as described herein and a transposase.

The transposase is not further limited and can be, for example, Sleeping Beauty transposase or PiggyBac transposase. The Sleeping Beauty transposase can be, e.g., represented by an amino acid sequence shown in SEQ ID NO: 45.

The transposable element is preferably integrated into the genome of the immune cells by the action of the transposase.

In an embodiment, the immune cells are lymphocytes, more preferably T cells or NK cells.

However, other immune cells, such as macrophages are also encompassed. Most preferably, the immune cells are T cells.

In a further embodiment, the T cell is a CD4⁺ T cell and/or a CD8⁺ T cell.

In a further embodiment of the invention, the blood sample is (or has been) obtained from a human subject, preferably a human subject diagnosed with cancer, preferably diagnosed with leukemia, such as AML.

In another embodiment, the invention provides a method for producing immune cells, comprising administering an expression vector encoding the siglec-6-binding polypeptide as described herein to a subject (in vivo gene transfer), see e.g. references [38]-[40], all incorporated by reference. Preferably, the expression vector for in vivo gene transfer is a lentiviral vector pseudotyped to transduce human immune cells (preferably T cells), or a nanoparticle containing a non-viral vector suitable for delivering the non-viral vector to human immune cells (preferably T cells).

The invention also relates to an immune cell (preferably lymphocyte, more preferably T cell) or a formulation of immune cells (preferably lymphocytes, more preferably T cells) obtainable by the method as described above.

Pharmaceutical Composition

The present invention also relates to a pharmaceutical composition comprising a plurality of immune cells (preferably lymphocyte, more preferably T cell) as described herein. The pharmaceutical composition may further comprise at least one pharmaceutically acceptable carrier. The pharmaceutical composition can optionally comprise a mixture of different cells, such as a mixture of CD4⁺ and CD8⁺ T cells.

In one embodiment of the invention, the pharmaceutical composition may be formulated as infusion solution comprising NaCl, glucose and human serum albumin in an amount of 0.45%, 2,5% and 1%, respectively.

Medical Uses

The present invention also relates to the immune cell or pharmaceutical composition as described herein for use as a medicament.

In an embodiment of the invention, the immune cell or pharmaceutical composition is for use in a method of treating cancer, wherein in said method the immune cell or pharmaceutical composition of the invention is to be administered to a subject (in need thereof), preferably a human subject.

The invention also relates to a method of treating cancer, comprising administering the immune cell or pharmaceutical composition of the invention to a subject, preferably a human subject.

In an embodiment of the invention, the immune cell or pharmaceutical composition is to be administered intravenously.

In an embodiment of the invention, the cancer is a siglec-6 expressing cancer, i.e. a cancer caused by abnormal cells expressing and displaying the siglec-6 protein on the cell surface. Preferably, the cancer is selected from the group consisting of leukemia, such as AML or CLL, MALT lymphoma or clonal mast cell disease and solid tumors such as thymoma. Preferably the cancer is leukemia, such as AML or CLL, and most preferably AML.

The (use in) the method of treating cancer preferably involves the elimination of cancer stem cells of said cancer by said immune cells. Such cancer stem cells are preferably leukemic stem cells, such as AML stem cells. Cancer stem cells are defined as a subset of cancer cells with enhanced tumorigenic potential, and/or capabilities of self-renewal and differentiation, and/or phenotypic, functional and/or genetic features that can be determined in order to distinguish them from non-cancer stem cells.

The (use in) the method of treating cancer also preferably does not involve the elimination of non-cancerous hematopoietic stem or progenitor cells by said immune cells. Hematopoietic stem or progenitor cells can be identified as CD34⁺ cells. Hematopoietic stem cells are typically CD38⁻ and hematopoietic progenitor cells are typically CD38⁺. Non-cancerous hematopoietic stem or progenitor cells are typically CD45*.

In light of this, the (use in) the method of treating cancer can further comprise monitoring the elimination of said cancer stem cells and/or of said non-cancerous hematopoietic stem or progenitor cells.

Thus, the (use in) a method of treating cancer can comprise monitoring the elimination of said cancer stem cells.

The (use in) a method of treating cancer can comprise monitoring the elimination of said non-cancerous hematopoietic stem or progenitor cells.

The (use in) a method of treating cancer can further comprise monitoring the elimination of said cancer stem cells and of said non-cancerous hematopoietic stem or progenitor cells. Elimination of cancer stem cells and/or non-cancerous hematopoietic stem or progenitor cells can be monitored, for example by bone marrow analyses that include flow cytometric analyses and other methods of phenotyping, including high-resolution flow cytometry for minimal residual disease (MRD) analyses, as well as next-generation sequencing and other methods of genotyping. Elimination of cancer stem cells and/or non-cancerous hematopoietic stem or progenitor cells can also be monitored by peripheral blood analyses that include blood counts and differential blood counts, as well as liquid biopsies and other methods of genotyping.

The phenotypic markers that are typically being used to identify AML LCS by flow cytometry include CD45, CD34 and CD38 (AML LSC phenotype: CD45^{dim}CD34⁺CD38⁻). Additional optional markers (and corresponding phenotype) that have been used to identify and/or characterize AML LSCs include: HLA-DR (+), CD25 (+), CD26 (+), CD32 (+), CD33 (+), CD36 (+), CD44 (+), CD45RA (+), CD47 (+), CD71 (+), CD90 (+), CD96 (+), CD99 (+), CD117 (+), CD123 (+), CD133 (+), CD135 (+), IL-1RAP (+), CD184 (+), CD305 (+), CD366 (+), CD371 (+) [35, 36, 37]. A cell being CD45dim means that the detectable surface expression of CD45 is lower than that of cells classified as CD45+. For example, (CD45dim) cancer stem cells may have a lower (mean) CD45 surface expression than healthy (CD45⁺) hematopoietic stem or progenitor cells.

Moreover, by not eliminating non-cancerous hematopoietic stem or progenitor cells, the need of conducting a bone marrow transplantation after the therapy using the modified immune cells can be avoided. Thus, in one embodiment, the (use in) the method of treating cancer does not involve allogeneic hematopoietic stem cell transplantation.

The treatment with the immune cells comprising the siglec-6-binding polypeptide also does not require depletion of said immune cells after the treatment. Thus, the invention also provides a (use in) the method of treating cancer as described herein that does not involve depletion of the immune cells after treatment.

Furthermore, in one embodiment, the (use in) the method of treating cancer does not involve additional conventional chemotherapy. Preferably, the (use in) the method of treating cancer does not involve additional chemotherapy after administration of the immune cells or the pharmaceutical composition and/or after the termination of the therapy with the immune cells or the pharmaceutical composition.

Conventional chemotherapy as used herein refers to the therapeutic use of chemotherapeutic agents that do not specifically target a given diseased cell (e.g. cancer cell) to be treated. Such chemotherapeutic agents include, for example, cytarabine and daunorubicin. Conventional chemotherapy as used herein does not refer to targeted therapies that are used to target a given diseased cell (e.g. cancer cell) more specifically. Such targeted therapies include, for example, the administration of a Bruton Tyrosine Kinase (BTK) inhibitor, such as ibrutinib; or an fms-like tyrosine kinase 3 (FLT3) inhibitor such as midostaurin; or an epigenetic therapy such as a hypomethylating agent (DNA methyltransferase inhibitor) such as 5-Azacitidin. Such targeted and/or epigenetic therapies can be used either in combination or as maintenance therapy with the immune cells comprising the siglec-6-binding polypeptide.

Thus, the (use in) a method of treating cancer can be a combination therapy, further comprising administration of, e.g., a targeted therapy such as a Bruton Tyrosine Kinase (BTK) inhibitor (e.g. ibrutinib); or an fms-like tyrosine kinase 3 (FLT3) inhibitor (e.g. midostaurin); or an epigenetic therapy such as a hypomethylating agent (DNA methyltransferase inhibitor) such as 5-Azacitidin either in combination or as maintenance therapy after the treatment with the immune cells comprising the siglec-6-binding polypeptide.

The treatment using immune cells binding siglec-6 is most effective against target cells expressing siglec-6. Therefore, the invention also provides a method of determining the expression level of siglec-6 on the surface of cancer cells from a (human) subject. The method is preferably an in vitro method. It is preferably conducted on a sample that has been obtained from a subject suspected of having cancer or diagnosed with having cancer. For example, the sample may be a (peripheral) blood sample or biopsy of the cancer to be treated.

The invention also provides a method of diagnosing cancer, preferably AML, the method comprising determining the expression level of siglec-6 on the surface of cancer cells from a (human) subject. The method is preferably an in vitro method. It is preferably conducted on a sample that has been obtained from a subject suspected of having cancer. For example, the sample may be a (peripheral) blood sample or biopsy of the cancer to be treated.

Moreover, in one embodiment, the (use in) the method of treatment as described further comprises determining, before treatment, the expression level of siglec-6 on the surface of the designated target cells, such as cancer cells of the cancer to be treated, e.g. AML.

The step of determining the expression level on siglec-6 is preferably conducted in vitro. It is preferably conducted on a sample that has been obtained from the subject to be treated. For example, the sample may be a (peripheral) blood sample or biopsy of the cancer to be treated.

A biopsy of a cancer (such as AML) can be, for example, a bone marrow biopsy or a tissue biopsy (e.g. of extracellular AML manifestation).

Consequently, in an embodiment, the (use in) the method of treatment as described further comprises administering the immune cells only if the designated target cells, such as cancer (e.g. AML) cells, express siglec-6.

Thus, the invention also provides a method of treating cancer, the method comprising:
1) determining the expression level of siglec-6 on cancer cells obtained from a subject;
2) administering an immune cell or pharmaceutical composition of the invention to said subject, wherein optionally the immune cell or pharmaceutical composition is administered only if siglec-6 is expressed on said cancer cells.

The invention also provides an immune cell or pharmaceutical composition for use in a method of treating cancer in a subject, the method comprising:
1) determining the expression level of siglec-6 on cancer cells obtained from a subject;
2) administering an immune cell or pharmaceutical composition of the invention to said subject, wherein optionally the immune cell or pharmaceutical composition is administered only if siglec-6 is expressed on said cancer cells.

Preferably, the cancer is selected from the group consisting of leukemia, such as AML or CLL, MALT lymphoma or clonal mast cell disease, preferably AML and CLL, and most preferably AML.

The skilled artisan is able to set suitable criteria for determining if siglec-6 is expressed on a given cell. For example, surface expression can be determined by flow cytometry as described herein. Briefly, the cells can be (surface-)stained in two separate samples, with a monoclonal antibody (mAb) against siglec-6 conjugated to a detectable (e.g. fluorescent) dye in the first sample and with an isotype control (i.e. a control monoclonal antibody not targeting siglec-6 that is of the same isotype as the siglec-6 mAb used) conjugated to the same detectable dye in the second sample. If the (mean) detectable intensity of the detectable dye (such as the mean fluorescence intensity, MFI) in the first sample is higher than in the second sample, the cells can be classified as expressing siglec-6. If the (mean) detectable (e.g. fluorescent) signal from the detectable dye in the first sample is the same or lower than in the second sample, the cells can be classified as not expressing siglec-6.

For example, the cells can be classified as expressing siglec-6 if the (mean) detectable intensity of the detectable dye (such as the MFI) in the first sample divided by the (mean) detectable intensity of the detectable dye (such as the MFI) in the second sample (such as the normalized mean fluorescence intensity (NMFI) that is calculated by dividing MFI obtained after staining with anti-siglec-6 mAb with MFI of isotype control) is greater than 1.0, at least 1.1, at least 1.2, at least 1.3, at least 1.4, at least 1.5 or at least 2, preferably at least 1.2.

The cells can also be classified as expressing siglec-6 if the value obtained by dividing the (mean) detectable intensity of the detectable dye (such as the MFI) in the first sample by the (mean) detectable intensity of the detectable dye (such as the MFI) in the second sample (such as the NMFI) is at least the same as the value obtained by dividing the (mean) detectable intensity of the detectable dye (such as the MFI) in a first sample (stained with siglec-6 mAb) using U937 cells by the (mean) detectable intensity of the detectable dye (such as the MFI) in a second sample (stained with isotype control) using MOLM-13 cells (such as the NMFI).

The pharmaceutical composition as described above comprising the modified T cells are stored at 2-8° C. The pharmaceutical composition is stable for (at least) 48 hours after formulation and ought to be administered to the patient within this period.

### EXAMPLES

Siglec-6 CAR-T cells as generated in the experimental section of the application relate to a non-limiting exemplified embodiment of the present invention.

Materials and Methods

Human Subjects

Human peripheral blood (PB) T-cells were obtained from buffy coat of healthy donors (HDs) and AML patients. G-CSF mobilized PB CD34+ cells were isolated from HD PB. Primary AML bone marrow (BM) and PB, and CLL PB samples were obtained after written informed consent.

Structure of a Siglec-6-Binding CAR Polypeptide

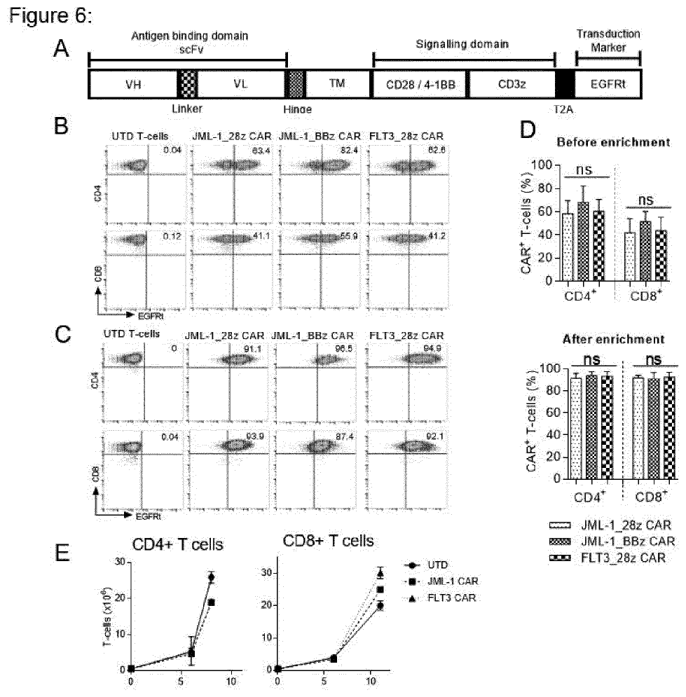

A schematic representation of the gene cassette as expected to be contained in a siglec-6 CAR T-cell is shown in FIG. 6.

The exemplary gene cassette comprising a nucleotide sequence encoding a siglec-6 CAR polypeptide also contains an optional truncated epidermal growth factor receptor (EGFRt) sequence, separated from the CAR sequence by a T2A ribosomal skip element to ensure translation of CAR and EGFRt into two separate proteins and stochiometric expression of both proteins on the T cell surface.

The EGFRt protein enables detection and selection of CAR-positive cells using the anti-EGFR monoclonal antibody cetuximab (trade name: Erbitux®). In addition, EGFRt opens the option for selective depletion of cells expressing EGFRt with cetuximab in the event of unmanageable toxicity. It was demonstrated in pre-clinical models that administration of cetuximab leads to depletion of CAR-T cells that express EGFRt within few days in vivo.

The structure and amino acid sequence of exemplary CARs are given in Table A.

TABLE A
Annotated sequence of exemplary gene
cassette.

| | | |
|---|---|---|
| | GM-CSF | MLLLVTSLLLCELPHPAFLLIP |
| | signal | (SEQ ID NO: 1; encoding |
| | peptide | DNA sequence |
| | | SEQ ID NO: 2) |
| Siglec-6 | JML-1 VH | KVQLLESGGGLVQPGRSLRLSCA |
| targeting | | ASGFTFDDYGMHWVRQAPGKGLE |
| element | | WVSGISWNSGSIGYADSVKGRFT |
| comprising | | ISRDNSKNTLYLQMNSLRAEDTA |
| VH- | | VYYCARGGQTIDIWGQGTMVTVSS |
| linker-VL | | (SEQ ID NO: 3; encoding |
| | | DNA sequence |
| | | SEQ ID NO: 4) |
| | linker | GGGGSGGGGSGGGGS |
| | | (SEQ ID NO: 5; encoding |
| | | DNA sequence |
| | | SEQ ID NO: 6) |
| | JML-1 VL | DIQMTQSPSSLSASVGDRVTITCR |
| | | ASQSISSYLNWYQQKPGKAPKLLI |
| | | YAASSLQSGVPSRFSGSGSGTDFT |
| | | LTISSLQPEDFATYYCQQSYSTPF |
| | | TFGPGTKVDIK |
| | | (SEQ ID NO: 7; encoding |
| | | DNA sequence |
| | | SEQ ID NO: 8) |
| Spacer | IgG4 | ESKYGPPCPPCP |
| domain | hinge | (SEQ ID NO: 9; encoding |
| from | | DNA sequence |
| IgG4 or | | SEQ ID NO: 10) |
| IgG3 | IgG3 | ELKTPLGDTTHTCPRCPEPKSCDT |
| | hinge | PPPCPRCP |
| | | ((SEQ IDNO: 11; encoding |
| | | DNA sequence |
| | | SEQ ID NO: 12) |
| | CD28 | MFWVLVVVGGVLACYSLLVTVAFI |
| | trans- | IFWV |
| | membrane | (SEQ ID NO:13; encoding |
| | | DNA sequence |
| | | SEQ ID NO: 14) |
| CD28 or | CD28 | RSKRSRGGHSDYMNMTPRRPGPTR |
| 4-1BB | costimu- | KHYQPYAPPRDFAAYRS |
| intracel- | latory | (SEQ ID NO: 15; encoding |
| lular | domain | DNA sequence |
| costimu- | 4-1BB | SEQ ID NO: 16) |
| latory | costimu- | KRGRKKLLYIFKQPFMRPVQTTQE |
| domain | latory | EDGCSCRFPEEEEGGCEL |
| | domain | (SEQ ID NO: 17; encoding |
| | | DNA sequence |
| | | SEQ ID NO: 18) |
| | CD3-zeta | RVKFSRSADAPAYQQGQNQLYNEL |
| | | NLGRREEYDVLDKRRGRDPEMGGK |
| | | PRRKNPQEGLYNELQKDKMAEAYS |
| | | EIGMKGERRRGKGHDGLYQGLSTA |
| | | TKDTYDALHMQALPPR |
| | | (SEQ ID NO: 19; encoding |
| | | DNA sequence |
| | | SEQ ID NO: 20) |
| Linkage | T2A | LEGGGEGRGSLLTCGDVEENPGPR |
| of | sequence | (SEQ ID NO: 21; encoding |
| optional | | DNA sequence |
| detect- | | SEQ ID NO: 22) |
| able | Signal | MLLLVTSLLLCELPHPAFLLIP |
| marker | peptide | (SEQ ID NO: 1; encoding |
| | | DNA sequence |
| | | SEQ ID NO: 2) |
| | EGFRt | RKVCNGIGIGEFKDSLSINATNIK |
| | | HFKNCTSISGDLHILPVAFRGDSF |
| | | THTPPLDPQELDILKTVKEITGFL |
| | | LIQAWPENRTDLHAFENLEIIRGR |
| | | TKQHGQFSLAVVSLNITSLGLRSL |
| | | KEISDGDVIISGNKNLCYANTINW |
| | | KKLFGTSGQKTKIISNRGENSCKA |
| | | TGQVCHALCSPEGCWGPEPRDCVS |
| | | CRNVSRGRECVDKCNLLEGEPREF |
| | | VENSECIQCHPECLPQAMNITCTG |
| | | RGPDNCIQCAHYIDGPHCVKTCPA |
| | | GVMGENNTLVWKYADAGHVCHLCH |
| | | PNCTYGCTGPGLEGCPTNGPKIPS |
| | | IATGMVGALLLLLVVALGIGLFM |
| | | (SEQ ID NO: 23; encoding |
| | | DNA sequence |
| | | SEQ ID NO: 24) |

CAR Construction

A codon-optimized targeting domain containing the V_{H} and the V_{L} segments of the JML-1 mAb (GeneArt ThermoFisher, Regensburg, Germany, SEQ ID NO: 25; codon-optimized DNA sequence SEQ ID NO: 26) was synthesized and fused to a CAR backbone comprising a short IgG4-Fc hinge spacer, a CD28 transmembrane domain, CD28 or 4-1BB costimulatory moiety, and CD3z (SEQ ID NO: 27 or 29; DNA sequence SEQ ID NO: 28 or 30), in-frame with a T2A element and EGFRt transduction marker (FIG. 6A) [14-16]. The entire transgene was encoded in a lentiviral vector epHIV7 and expressed under control of an EF1/HTLV hybrid promotor [16]. CARs specific for FLT3 (clone 4G8), CD19 (clone FMC63) and CD123 (clone 32716) proteins with CD28 or 4-1BB costimulatory moiety [14,15,17-19] were used for controls in this study.

Primary AML and CLL Cells

AML patients' BM and PB were processed for mononuclear cell isolation using density-gradient centrifugation (Biocoll®, Merck Millipore). The samples with less than 1 mL volume were directly processed for flow cytometry analysis after red blood cell lysis. If possible, cytotoxicity analysis was performed directly after this step or else cells were frozen down and kept at −80° C. until the experiment was performed. Thawed primary AML cells were maintained in RPMI-1640 supplemented with 10% human serum, 2 mM glutamine, 100U/mL penicillin/streptomycin, and a cytokine cocktail including IL-4 (10001U/mL), granulocyte macrophage colony-stimulating factor (GM-CSF) (10 ng/mL), stem cell factor (5 ng/mL) and tumor necrosis factor (TNF)-α (10 ng/mL) (cytokines from Miltenyi Biotec, Germany). Fresh primary CLL samples were analyzed by flow cytometry and subsequently cells were frozen for cytotoxicity assays.

Tumor Cell Lines

Human tumor cell lines U937 (ATCC CRL-1593.2), MOLM13 (ACC 554), MV4;11 (ACC 102), K562 (ACC 10), TF-1 (ACC 334), Kasumi-1 (ACC 220) were purchased from American Type Culture Collection (ATCC, USA) or DSMZ (Deutsche Sammlung von Mikroorganismen und Zellkulturen, Braunschweig, Germany). Cells were cultured in RPMI-1640 supplemented with 10% fetal calf serum (FCS), 2 mM glutamine and 100 U/mL penicillin/streptomycin. To enable detection of the cells by flow cytometry and by bioluminescence imaging, the inventors transduced all cell lines with a lentiviral vector encoding a firefly luciferase (ffluc)_green fluorescent protein (GFP) transgene, enriched GFP⁺ cells by FACS sorting before utilizing in in vitro or in vivo studies.

K562/siglec-6 was generated by electroporation of full-length human SIGLEC-6 gene in K562 cells. For this, full-length siglec-6 DNA (SEQ ID NO: 46) was cloned in pT2HB vector backbone and nucleofected with SB100x minicircle DNA vector using 4D nucleofector (Lonza, Switzerland). Nucleofected cells were stained with APC conjugated anti-siglec-6 mAb and siglec-6-positive cells were enriched by FACS based cell sorting.

Generation of CAR-Modified T-Cells and In Vitro Analyses

Preparation of CAR-modified T-cells, analyses of CAR-T function in vitro, and colony formation assays were performed as previously described [14, 15, 17, 19, 20]. Patient-derived CD4⁺ and CD8⁺ T-cells were isolated from PB by positive selection, transduced with CAR encoding lentiviruses, and CAR⁺ T-cells were enriched using biotinylated anti-EGFR mAb (see above) and anti-biotin beads (Miltenyi), prior to expansion using a rapid expansion protocol [15, 20].

Colony Formation Assay

G-CSF-mobilized human CD34⁺CD38⁻ and CD34⁺CD38+peripheral blood HSC/P cells from PB were seeded at 5×10³ cells/well in duplicate wells and incubated with JML-1-CAR or CD123-CAR T-cells or untransduced T-cells at an E:T ratio=5:1. After 24 hours, 1/5^{th} of cell suspension was plated onto methyl cellulose-based medium (Methocult opti H4034, Stem Cell Technologies, Cambridge, MA) in 6-well plates (Smartdish™ plate, StemCell Technologies). Colonies were evaluated using established criteria according to the manufacturer's instructions and counted under an optical microscope 14 days later.

Flow Cytometric Analyses

Mononuclear cells from healthy donors or AML patients were stained with 1 or more of the following conjugated mAbs: CD3, CD4, CD8, CD19, CD33, CD34, CD38, CD45, CD56, CD123, CD135 (FLT3), CD327 (siglec-6), CD371 (CLL-1) with matched isotype controls, and 7-AAD for live/dead cell discrimination (Miltenyi biotec, Bergisch-Gladbach, Germany; BD, Heidelberg, Germany; Biolegend, London, UK). PB mononuclear cells (PBMCs) from CLL patients were stained with the 1 or more of following conjugated mAbs: CD5, CD10, CD19, CD20, CD27, CD38, CD327 (Miltenyi biotec, Bergisch-Gladbach, Germany; BD, Heidelberg, Germany; Biolegend, London, UK). Untransduced or CAR-transduced T-cells were stained with one or more of the following conjugated mAbs: CD3, CD4, CD8, and 7-AAD (Miltenyi biotec/BD/Biolegend). An anti-EGFR antibody (ImClone Systems Inc.) that had been conjugated to AF647 in-house (EZ-Link™Sulfo-NHS-SSBiotin, ThermoFisher Scientific, IL; according to the manufacturer's instructions) was used to detect CAR T-cells. Cells were acquired on FACSCanto (BD) for flow cytometry analyses and data analysis was performed using FlowJo software v9.0.2 (Treestar, Ashland, OR). Primary AML blasts were stained with mAb against CD45, CD34, CD38, CD123, CD33, FLT3, Siglec-6, CLL-1, and CD117. LSC were identified as CD45^{dim}CD34*CD38⁻ cells. The normalized mean fluorescence intensity (NMFI) was calculated by dividing the MFI obtained by flow cytometry after staining with a specific mAb (e.g. anti-siglec-6) through the MFI obtained by flow cytometry after staining with an isotype control.

Siglec-6-Expression Analysis by Flow Cytometry

Expression of siglec-6 (CD327) was assessed using APC-conjugated mouse-anti-human-siglec-6 mAb (Clone 767329, R&D Systems, USA) or REAfinity™ anti human siglec-6 (clone REA852, Milteny biotec, Germany) and mouse IgG1 isotype control (R&D Systems, USA) or REA Control Antibody (S), human IgG1 (Milteny biotec, Germany). Briefly, 1×10⁶ cells were washed, re-suspended in 100 μL PBS/0.5% fetal calf serum, blocked with human IgG when R&D systems mAB was used (Jackson ImmunoResearch, USA) at 42C for 20 minutes and stained with anti-human siglec-6 mAb or isotype for 30 minutes at 4° C.

In Vivo Experiments with U937 Xenograft Model

The competent Institutional Animal Care and Use Committees evaluated and approved all in vivo experiments. NOD.Cg-Prkdcscidll2rgtm1Wj/SzJ (NSG) mice (female, 6-8 weeks old) were purchased from Charles River (Sulzfeld, Germany). Mice were inoculated with 2×10⁶ ffluc_GFP⁺ U937 cells. Mice were randomly allocated to different treatment groups, and injected using a split CAR T-cell dosing strategy, with doses administered on day 6 and on day 21 via tail vein. Each dose contained 5×10⁶ T-cells (i.e. 2.5×10⁶ CD4⁺ and 2.5×10⁶ CD8+ in 200 μL of PBS/0.5% FCS). PB was obtained at regular intervals to analyze the frequency of tumor cells and transferred T-cells. Bioluminescence imaging (BLI) was performed weekly after intraperitoneal administration of D-luciferin substrate (0.3 mg/g body weight) (Biosynth, Staad, Switzerland) using an IVIS Lumina imaging system (PerkinElmer, Waltham, Massachusetts). Bioluminescence images were analyzed using Living Image software (PerkinElmer).

In Vivo Experiments with MOLM-13 Xenograft Model

The competent Institutional Animal Care and Use Committees evaluated and approved all in vivo experiments. NOD.Cg-Prkdcscidll2rgtmlWj/SzJ (NSG) mice (female, 6-8 weeks old) were purchased from Charles River (Sulzfeld, Germany). Mice were inoculated with 1×10⁶ ffluc_GFP⁺ MOLM-13 cells. Mice were randomly allocated to different treatment groups, and injected using a split CAR T-cell dosing strategy, with doses administered on day 4 and on day 7 via tail vein. Each dose contained 5×10⁶ T-cells (i.e. 2.5×10⁶ CD4⁺ and 2.5×10⁶ CD8+ in 200 μL of PBS/0.5% FCS). PB was obtained at regular intervals to analyze the frequency of tumor cells and transferred T-cells. Bioluminescence imaging (BLI) was performed weekly after intraperitoneal administration of D-luciferin substrate (0.3 mg/g body weight) (Biosynth, Staad, Switzerland) using an IVIS Lumina imaging system (PerkinElmer, Waltham, Massachusetts). Bioluminescence images were analyzed using Living Image software (PerkinElmer).

Flow Cytometry-Based Cytotoxicity Assay

The cytolytic activity of JML-1-CAR, FLT3-CAR, CD19-CAR or untransduced T-cells against primary AML blasts and CLL cells was analyzed in a FACS-based cytotoxicity assay. T-cells and primary cells were seeded into 96-well plates at effector: target (E:T) ratios ranging from 10:1 to 2.5:1 with 10⁴ target cells per well. Co-cultured cells were stained after 4 or 24-hour co-culture for flow analysis with following mAbs: For AML samples, anti-CD3/anti-CD33/anti-CD34/anti-CD45/anti-EGFRt mAbs and for CLL samples, anti-CD3/anti-CD5/anti-CD20/anti-CD19/anti-CD45 were used to distinguish CAR T-cells and target cells. 7-AAD was used to discriminate live and dead cells. To quantitate the number of residual live AML cells, 123-counting beads (e-bioscience, San Diego, CA) were used according to the manufacturer's instructions. Flow analyses were done on a FACS Canto II (BD) and data analyzed using FlowJo software (Treestar).

Statistical Analyses

Statistical analyses were performed using Prism software v6.07 (GraphPad, San Diego, California). Student's t-test (Unpaired) was used to analyze data obtained in in vitro and in vivo experiments. The differences in survival observed in in vivo experiments were analyzed using Logrank (Mantel-Cox) test. P value with difference <0.05 were considered statistically significant.

Results

JML-1-CAR T-Cells Recognize and Eliminate Siglec-6⁺ AML Cell Lines

The inventors generated CD4⁺ and CD8+JML-1-CAR T-cells from healthy donors (HD) (n >5).

For this purpose, the inventors used single-chain fragment variable (scFv) derived from fully human JML-1- mAb10 and linked it to the CD3i signaling domain and CD28 or 4-1BB costimulatory domains (FIG. 6A). The inventors lentivirally transduced T-cells (multiplicity of infection=3) and observed transduction efficiency of 38.9-82.4% in CD4⁺ and 31.7-66.2% in CD8⁺ JML-1-CAR T-cells (FIG. 6B-D). Before expansion and functional testing of JML-1-CAR T-cells, the inventors performed enrichment step using the EGFRt selection marker that yielded >85% CAR⁺ T-cells (FIG. 6C-D). CD4⁺ and CD8+JML-1-CAR T-cells expanded similar to FLT3-CAR T-cells after bead stimulation and lentivirus transduction (FIG. 6E).

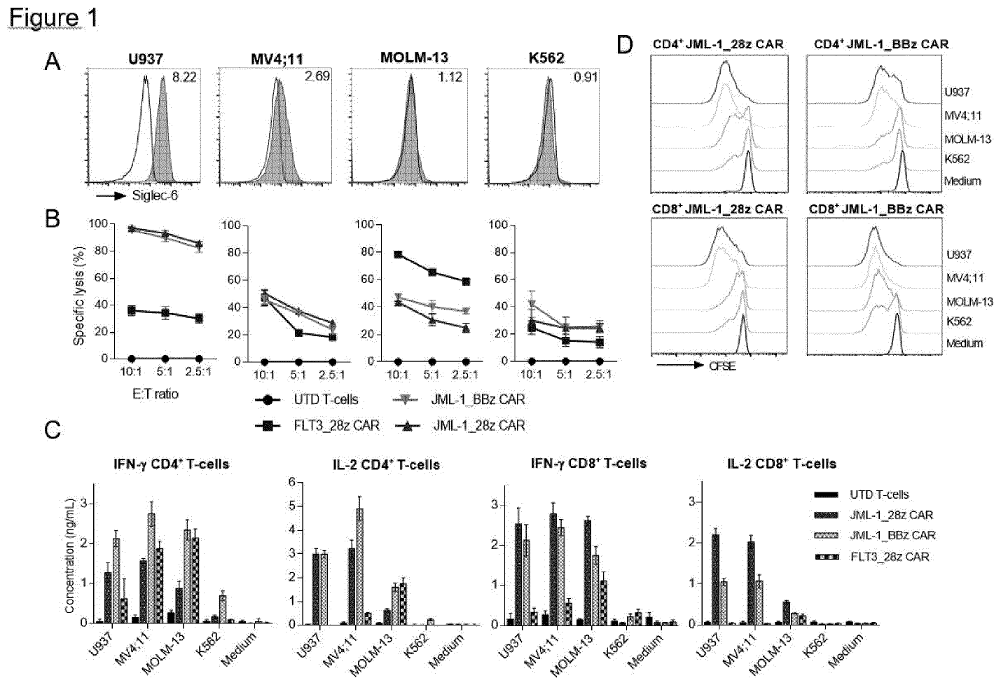

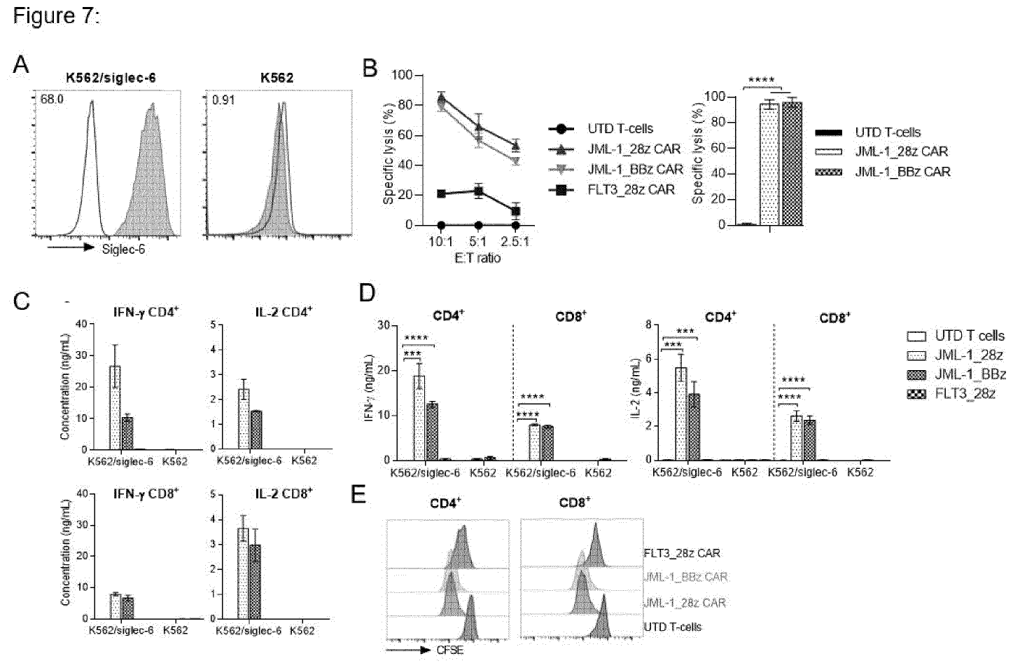

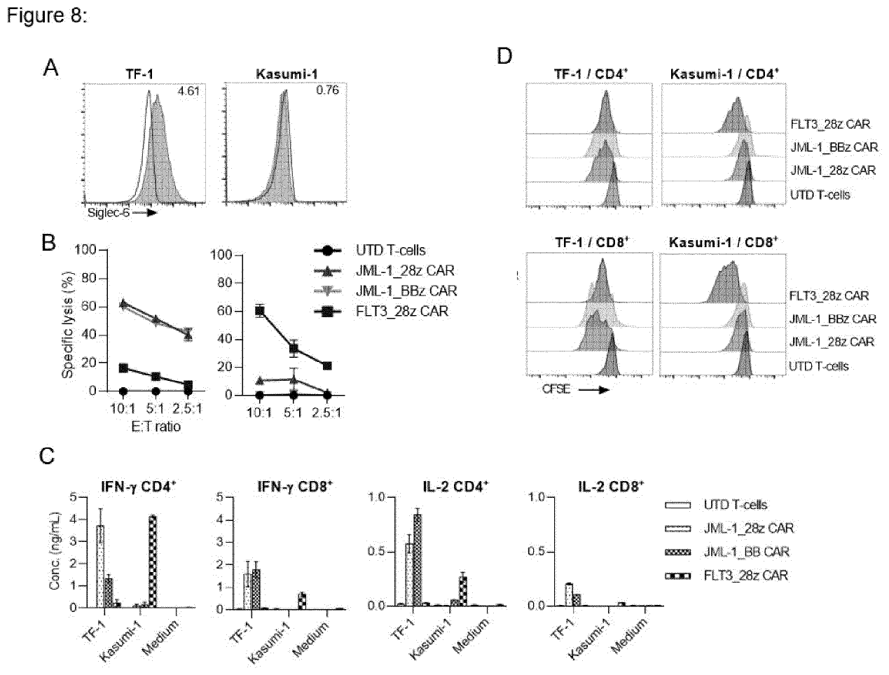

Next, the inventors engineered K562 cells to stably express siglec-6 (K562/siglec-6, FIG. 7A) and confirmed specific recognition of cell surface siglec-6 by JML-1-CAR T-cells on native K562 (siglec-6-negative) and K562/siglec-6 cells (FIG. 7B-E). Then, the inventors evaluated siglec-6-expression on different AML cell lines and observed variable siglec-6-expression levels (very high to low, normalized MFI=8.22 to 1.12) (FIG. 1A, FIG. 8A). Next, the inventors assessed recognition of siglec-6 by CD8⁺ JML-1-CAR T-cells using the AML cell lines U937 and TF-1 (high expression), MV4;11 (moderate expression), MOLM-13 (weak expression) and K562 and Kasumi-1 (no expression), and confirmed high-level of specific cytolytic activity of both JML-1_28z and JML-1_BBz CAR T-cells against siglec-6-positive cell lines (FIG. 1B, FIG. 8B). Notably, the specific lysis and kinetic of lysis correlated with antigen density on target cells (R²=0.57, p=0.01) (FIG. 1A-B, 2C, FIG. 8A-B). Of note, based on previous studies, the inventors selected analogously designed and functionally optimal FLT3 CAR (CD28z) as control for the assays. Both CD4⁺ and CD8+JML-1-CAR T-cells produced high levels of effector cytokines (i.e. IFN-γ and IL-2) and underwent productive proliferation after co-culture with siglec-6-positive AML cell lines, while the inventors only observed background reactivity in control T-cells and after exposure to antigen negative cell lines K562 and Kasumi-1 (FIGS. 1C-D, FIG. 8C-D).

Taken together, the data show that T-cells expressing the JML-1-CAR with CD28 or 4-1BB co-stimulatory domains show antigen-specific potent anti-leukemia reactivity against AML cell lines in vitro.

Siglec-6 is Highly and Uniformly Expressed on Primary AML Blasts, Including AML Leukemic Stem Cells/JML-1-CAR T-Cells Recognize and Eliminate Primary AML Cells In Vitro

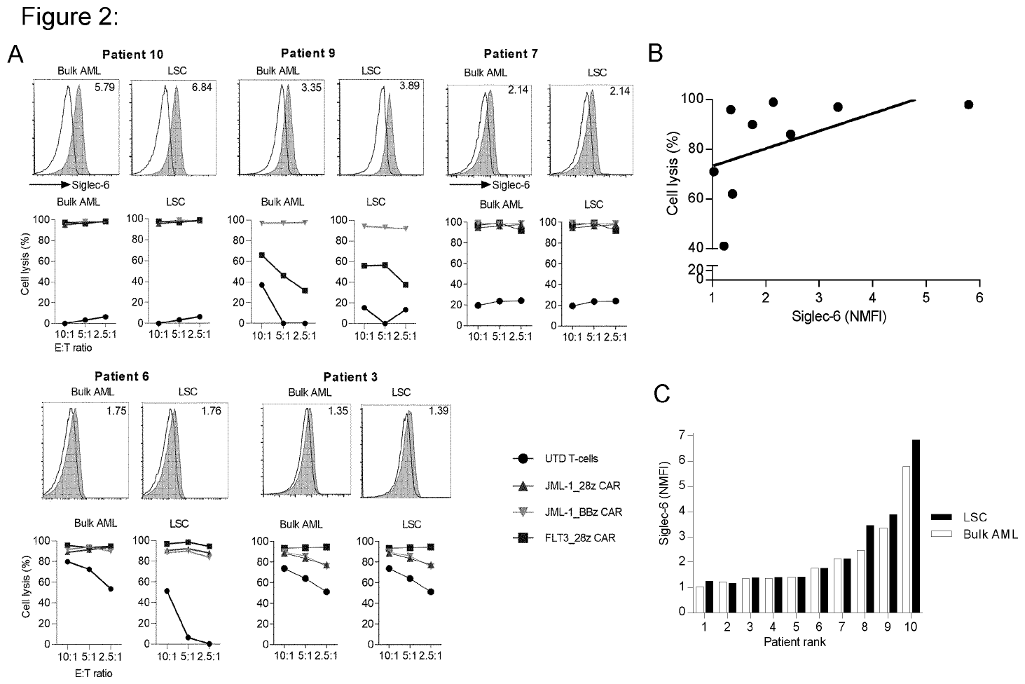

The inventors evaluated siglec-6-expression on primary AML blasts from n=10 adult AML patients. This patient cohort comprised patients with newly diagnosed AML, relapsed/refractory AML and secondary AML. Further, the patient cohort comprised patients and AML with various molecular and cytogenetic abnormalities (Table-1). The inventors found siglec-6 to be uniformly expressed on AML blasts in each of the patients (10/10), and ranked the patients according to the expression level based on normalized MFI (FIG. 2A, Table-1).

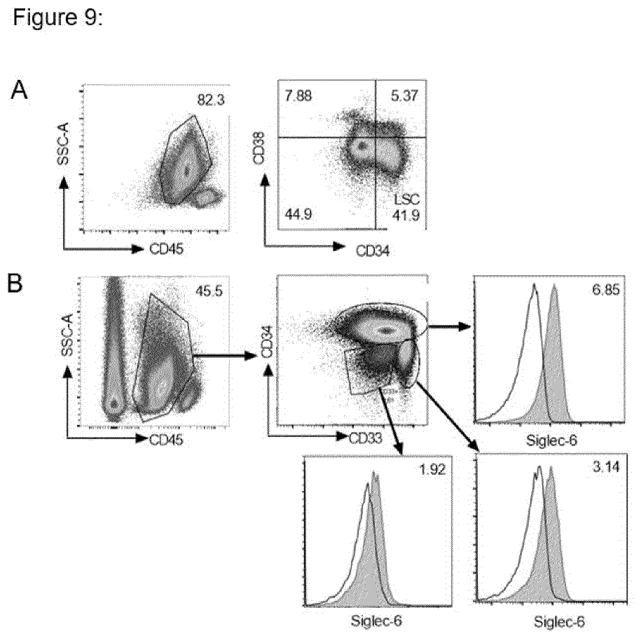

Intriguingly, the inventors also found uniform siglec-6 expression on the subpopulation of AML leukemia stem cells (LSCs) in each of the patients. Even more intriguingly, the expression level of siglec-6 was similar or even higher in the subpopulation of AML LSCs compared to the ‘bulk’ population of AML blasts (FIG. 2A, Table-1, FIG. 9A). The inventors also detected siglec-6-expression across heterogeneous AML blasts populations within the same patient, indicating that targeting siglec-6 will lead to complete and definitive elimination of AML blasts, and therefor provide an effective and potentially even curative treatment (FIG. 9B).

To assess recognition of primary ‘bulk’ AML blasts and AML leukemic stem cells, the inventors performed cytolysis experiments with CD8+JML-1-CAR T-cells that were derived from a healthy donor. The inventors observed high-levels of cytolytic activity by JML-1-CAR T cells against primary ‘bulk’ AML blasts and AML leukemic stem cells (FIG. 2A,B and Table-1). Importantly, even though leukemic stem cells are known to possess greater intrinsic resistance to conventional anti-AML treatments, the inventors observed that the cytolysis of ‘bulk’ AML blasts and AML LSCs by JML-1-CAR T-cells was similar, and that AML leukemic stem cells were rapidly eliminated. The cytolytic activity of JML-1-CAR T-cells with CD28 vs. 4-1BB costimulatory domain (JML-1_28z and JML-1_BBz CAR-T cells) against AML LSCs was similarly potent (FIG. 2A).

Taken together, the data show that siglec-6 is highly and uniformly expressed in primary AML blasts in patients with various AML disease subtypes. The data also show that siglec-6 is highly expressed in AML LSCs with an expression level that is similar or even higher compared to the ‘bulk’ AML blast population. The data further show that targeting siglec-6 confers specific and potent anti-AML activity and—on example of JML-1-CAR T-cells—leads to specific and potent elimination of bulk AML blasts and AML leukemic stem cells.

TABLE 1
Expression of siglec-6 in primary AML blasts and anti-AML reactivity of JML-1-CAR T-cells. NMFI: The normalized
mean fluorescence intensity (NMFI) is calculated by dividing the MFI obtained by flow cytometry after
staining with anti-siglec-6 mAb through the MFI obtained by flow cytometry after staining with an isotype control.
The absolute lysis of primary AML blasts was analyzed in a flow cytometry-based assay after a 24-hour co-culture
with JML-1_BBZ CAR T-cells at an 2.5:1 effector to target cell ratio.

| | NMFI | | Absolute | AML blasts | | | | | |
|---|---|---|---|---|---|---|---|---|---|
| | bulk | NMFI | lysis of | in bone | | | | | |
| Patient | AML | AML | bulk AML | marrow | | Disease | Secondary | Molecular | |
| rank | blasts | LSCs | blasts (%) | (%) | Age | status | AML | biology | Cytogenetics |
| 1 | 1.03 | 1.26 | 71 | 89.5 | 74 | Diagnosis | No | FLT3 (TKD | −Y, |
| | | | | | | | | mutations) | del(21)(q21q22), +13 |
| 2 | 1.22 | 1.17 | 41 | 48.5 | 36 | NA | NA | NA | NA |
| 3 | 1.35 | 1.39 | 96 | 67.5 | 59 | Diagnosis | NA | NA | NA |
| 4 | 1.36 | 1.40 | 62 | 89 | 59 | Diagnosis | NA | NA | NA |
| 5 | 1.42 | 1.42 | NA | 93.0 | 39 | Diagnosis | No | CBFB_MYH11/ | Normal |
| | | | | | | | | inv16 | |
| 6 | 1.75 | 1.76 | 90 | 79.4 | 71 | Diagnosis | No | DNMT3A, FLT3 | Normal |
| | | | | | | | | (ITD), RUNX1 | |
| 7 | 2.14 | 2.14 | 99 | 46 | 54 | Relapse | No | RUNX1 | t(12; 17)(p13; q11) |
| | | | | | | | | | .ish, t(12; 17)(NF1-; |
| | | | | | | | | | NF1-) |
| 8 | 2.47 | 3.46 | 86 | 53.8 | 76 | Relapse | Yes | DNMT3A, | Normal |
| | | | | | | | | TET2, TP53 | |
| 9 | 3.35 | 3.89 | 97 | 95.1 | 81 | Diagnosis | Yes | NA | NA |
| 10 | 5.79 | 6.84 | 98 | 46 | 52 | Diagnosis | No | — | t(4; 13)(q12; q13) |

Abbreviations:
FLT3: FMS-like Tyrosine Kinase 3,
ITD: internal tandem duplication,
TKD: tyrosine kinase domain,
NPM1: Nucleophosmin 1,
CEBPα: CCAAT/enhancer-binding protein alpha,
DNMT3A: DNA (cytosine-5)-methyltransferase 3A,
TET2: Tet methylcytosine dioxygenase 2,
TP53: Tumor protein p53,
RUNX1: Runt-related transcription factor 1,
MLL: mixed lineage leukemia,
PDGFRa: platelet-derived growth factor receptor A

Patient Derived JML-1 CAR-T Cells Potently Eliminate Autologous AML Blasts

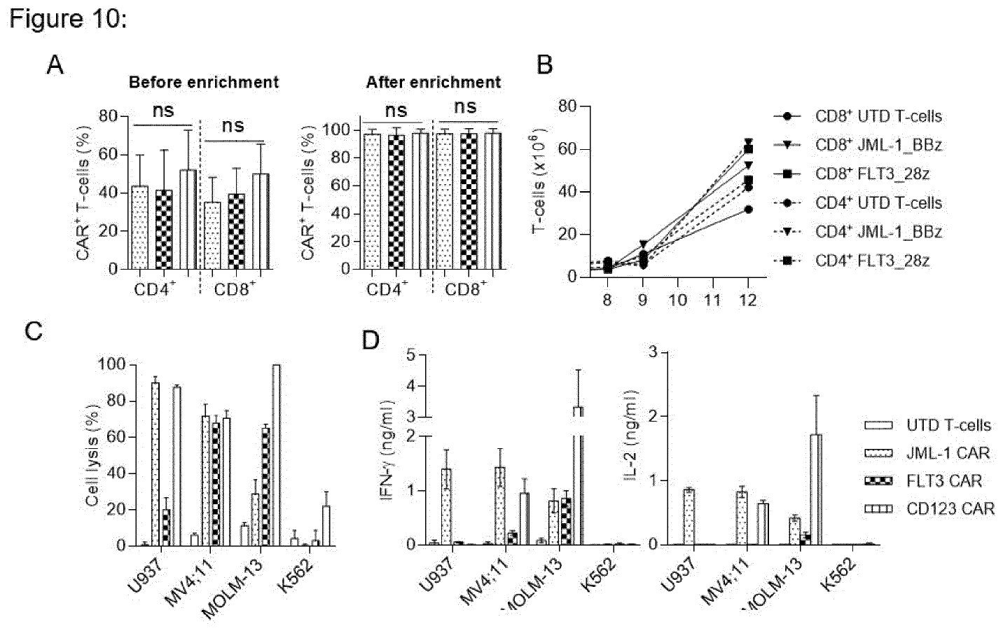

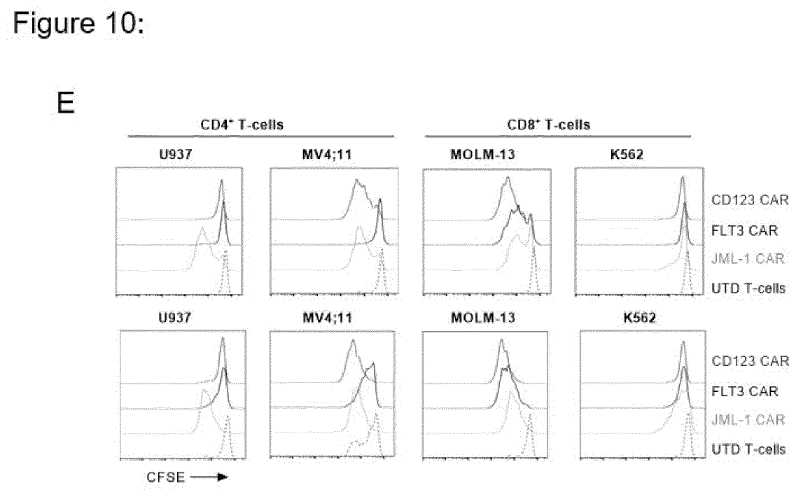

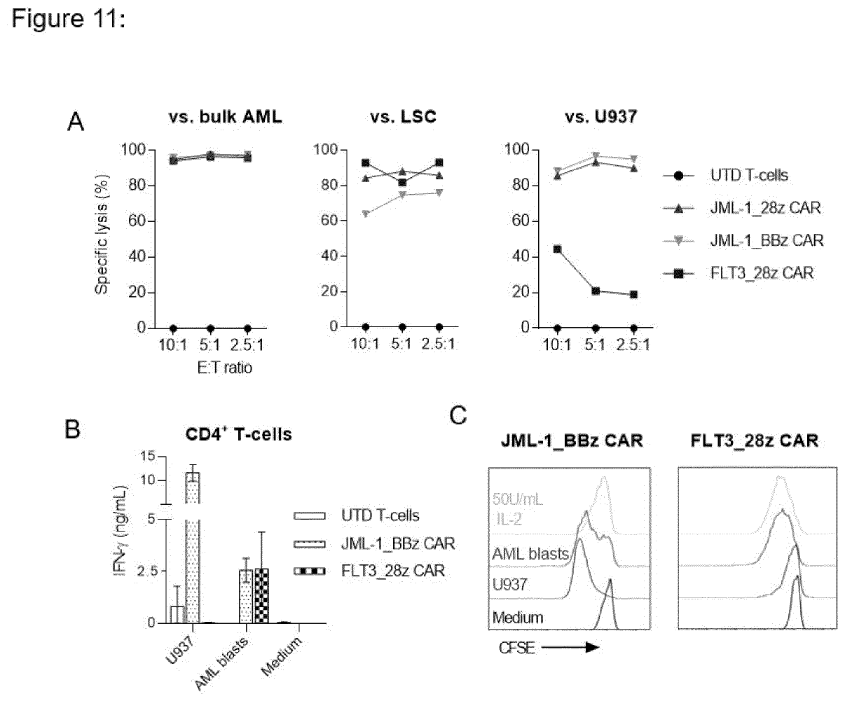

Next, the inventors generated JML-1-CAR T-cells from AML patients with newly diagnosed (n=2) and previously treated AML (MRD+, n=1) and assessed their anti-leukemia activity against autologous AML blasts. The inventors observed transduction efficiency of 24.9-50.0% in CD4⁺ and 20.4-43.0% in CD8⁺ T-cells and enriched cells were >95% CAR⁺ T-cells (FIG. 10A). Patient-derived CD4⁺ and CD8+JML-1-CAR T-cells expanded 40-60 fold within 12 days of culture (FIG. 10B) and showed potent anti-leukemia reactivity against siglec-6-positive cell lines in vitro (FIG. 10C-E). When co-cultured with autologous leukemic blasts, JML-1-CAR T-cells showed near to complete elimination of AML blasts in 24 hours (FIG. 11A), produced significant levels of IFN-γ and showed extensive proliferation (FIG. 11B-C). Again, JML-1-CAR T-cells conferred similarly potent cytolytic and cytotoxic activity against ‘bulk’ AML blasts and AML leukemic stem cells (FIG. 11A). Control non-CAR modified T-cells from the same respective patient did show any discernable reactivity in these functional assays. In conclusion, patient-derived JML-1-CAR T-cells are highly responsive to siglec-6-positive autologous AML blasts and AML cell lines.

JML-1-CAR T-Cells Eradicate Aggressive Systemic Acute Myeloid Leukemia In Vivo

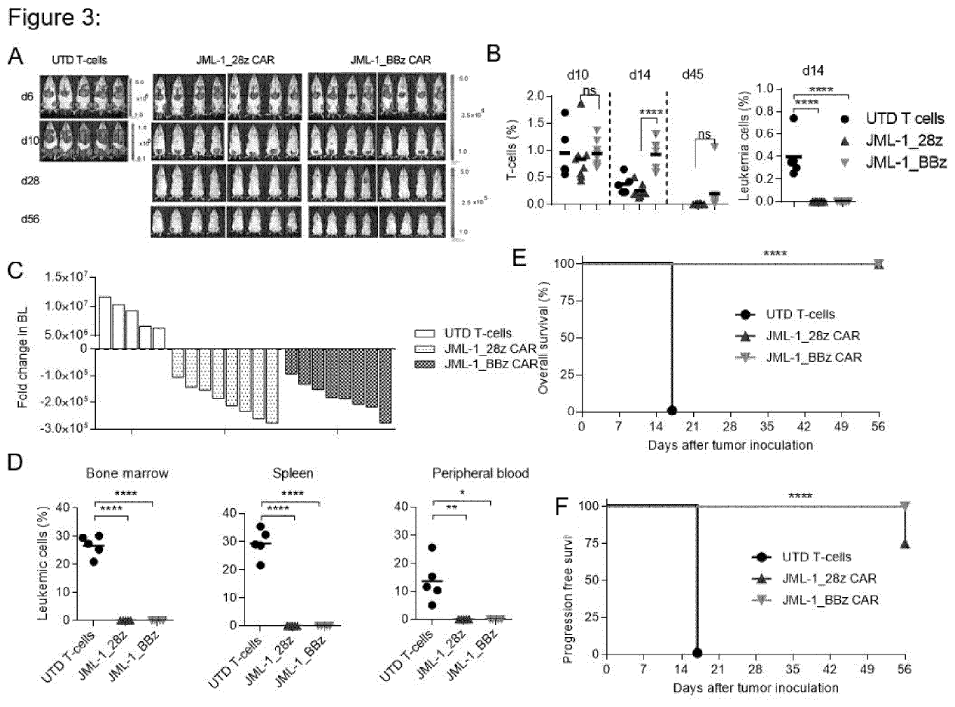

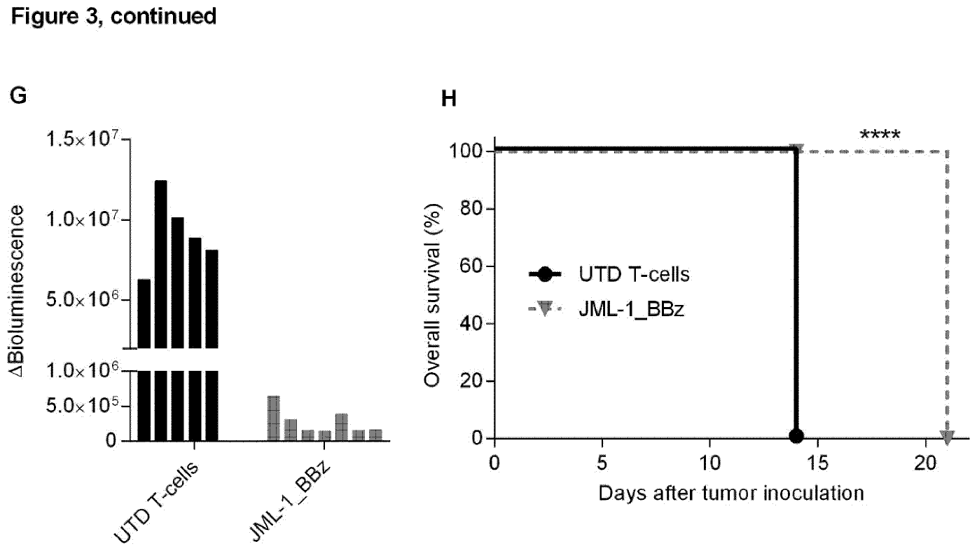

The inventors evaluated the anti-leukemia efficacy of JML-1-CAR T-cells in AML xenograft models using immunodeficient NSG mice. The inventors inoculated female NSG mice with ffLuc⁺GFP⁺ U937 leukemic cells and observed systemic engraftment of tumor cells in BM and spleen of mice after 6 days of tumor inoculation by BLI analysis (FIG. 3A). Then, the inventors treated mice with 5×10⁶ JML-1_28z or JML-1_BBz-CAR T-cells, or untransduced T-cells (CD4⁺:CD8⁺ ratio=1:1). The inventors observed engraftment, robust expansion and persistence of JML-1-CAR T-cells (FIG. 3B, left). Notably, JML-1_BBz CAR-T cells expanded and persisted significantly higher than JML-1_28z CAR T-cells in PB (FIG. 3B, left) and the inventors observed rapid clearance of leukemic cells from PB within 7 days after JML-1-CAR T-cell treatment (FIG. 3B, right). Moreover, all mice treated with JML-1-CAR T-cells showed rapid regression of leukemia while all mice treated with untransduced T-cells showed increasing leukemia burden (FIG. 3A, C).

At the end of the observation period, the inventors observed leukemia free BM, spleen and PB by flow cytometry, and confirmed sustained complete remission of AML in mice treated with JML-1-CAR T-cells, whereas progressive, deleterious leukemia was observed in all mice that had been treated with control T-cells (FIG. 3D, FIG. 11).

The inventors observed significantly higher overall survival among groups of mice treated with JML-1-CAR T-cells compared to control T-cells (FIG. 3E) and superior progression free survival in mice treated with JML-1-CAR T-cells compared to control T-cells (FIG. 3F).

The inventors also evaluated the anti-leukemia efficacy of JML-1-CAR T-cells in immunodeficient NSG mice that had been inoculated with MOLM-13 cells (low Siglec-6-expression). Treatment with Siglec-6-CAR T-cells conferred a significant anti-leukemia effect (FIG. 3G) and led to a significant survival benefit (FIG. 3H), but was less effective compared to the NSG/U937 model (high Siglec-6-expression).

In conclusion, these data demonstrate that targeting siglec-6 in AML confers potent anti-leukemia activity in vivo. The data also show that JML-1-CAR T-cells confer potent anti-leukemia efficacy and induce durable complete remissions of AML in vivo.

Human Hematopoietic Stem and Progenitor Cells do not Express Siglec-6 and are Preserved after Exposure to JML-1-CAR T-Cells In Vitro

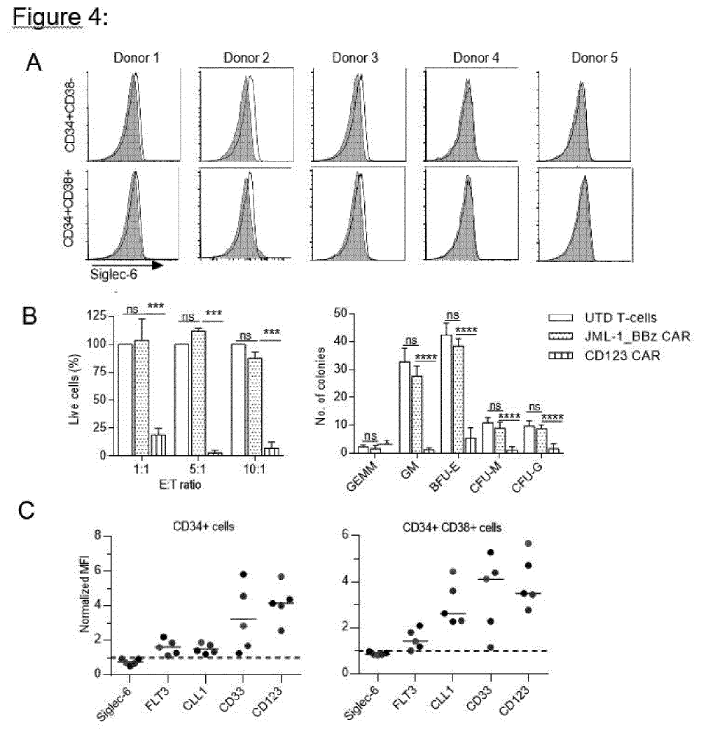

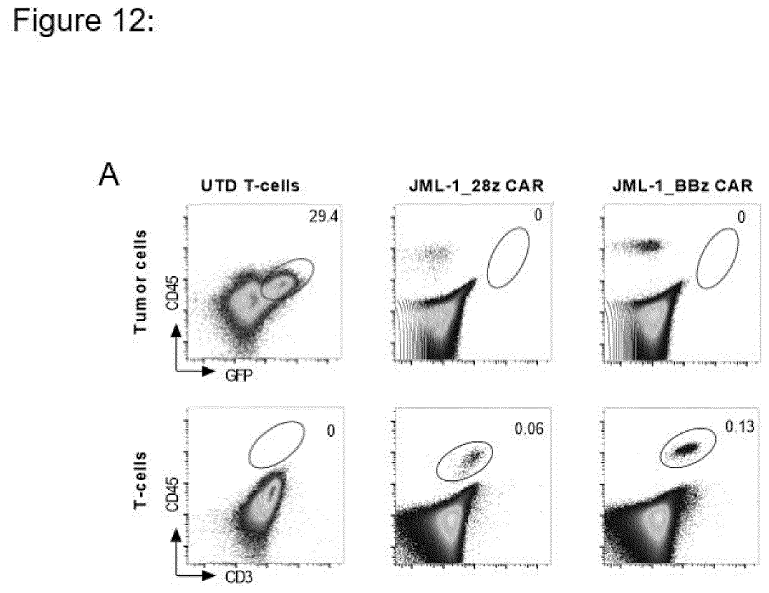

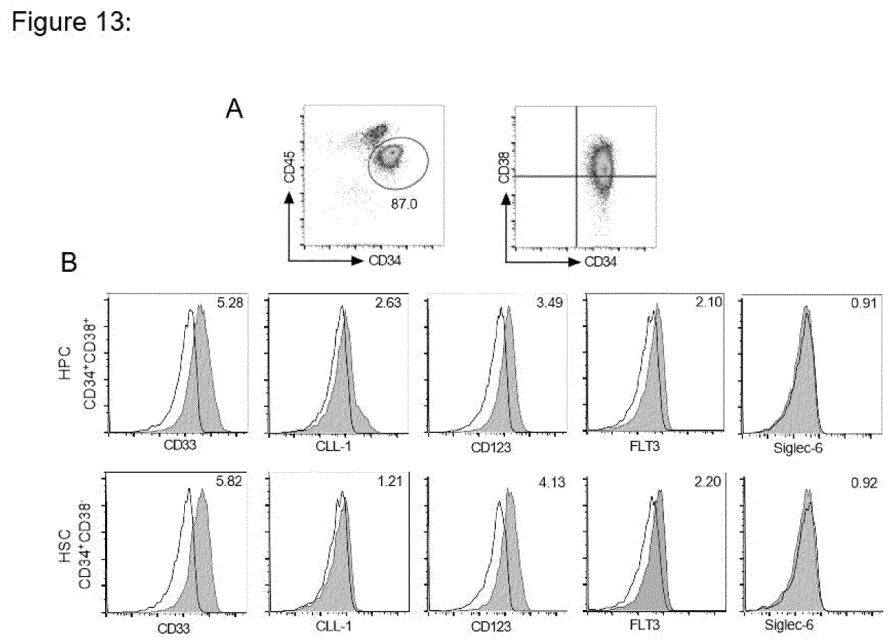

The inventors sought to evaluate on-target off-tumor effect of JML-1-CAR T-cells on normal hematopoietic stem and progenitor cells (HSC/P). First, the inventors assessed siglec-6-expression on G-CSF mobilized PB derived CD34⁺CD38⁻ HSC and CD34⁺CD38+HSPC from HD (n=5). The inventors observed lack of siglec-6-expression on HSC and HSPC in all n=5 HD analyzed by flow cytometry (NMFI <1.0, FIG. 4A). Next, the inventors co-cultured CD8+JML-1-CAR T-cells with HSC/P and assessed in vitro recognition of target cells. The inventors used CD123-CAR T-cells as a positive control in the assays due to its reported myeloablative effect [19]. The inventors observed that JML-1-CAR T-cells did not lyse normal HSC/P, while CD123-CAR T-cells rapidly eliminated the majority of HSC/P after 24 hours (FIG. 4B, left). To evaluate colony forming ability of residual HSC/P in vitro, colony formation assay was performed using residual HSC/P after 24-hour co-culture with JML-1-CAR or CD123-CAR T-cells. HSC/P treated with JML-1-CAR T-cells show comparable colony formation to HSC/P exposed to untransduced T-cells (FIG. 4B, right). As expected, the inventors observed a small number of erythroid colonies, while formation of myeloid colonies was completely ablated when exposed to CD123-CAR T-cells (FIG. 4B, right). Next, the inventors compared siglec-6 expression on HSC/P to other candidate CAR target antigens in AML (FLT3, CLL1, CD33 and CD123). The inventors observed absence of siglec-6 expression on healthy HSC/P in all five HD. In contrast, there was strong expression of FLT3, CLL1, CD33 and CD123 on healthy HSC/P in all five HD (FIG. 4C, FIG. 12A-B).

Taken together, these data show that siglec-6 is a unique AML target antigen in that it is not expressed on normal HSC/P. The data also show that normal HSC/P are not recognized by JML-1-CAR T-cells. These data suggest that targeting siglec-6 will not induce myeloablation in humans.

Malignant B-Cells in B-CLL Express Siglec-6 and are Eliminated by JML-1-CAR T-Cells

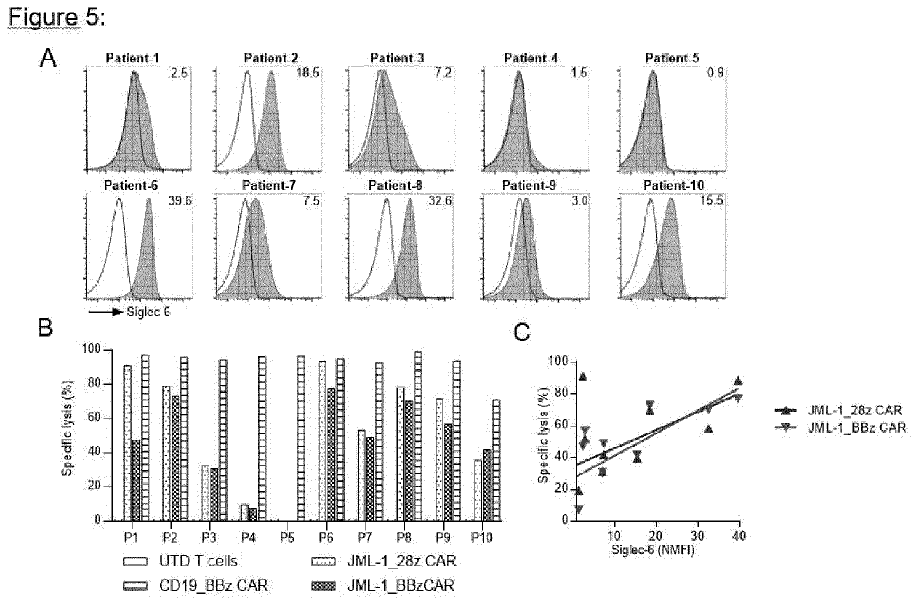

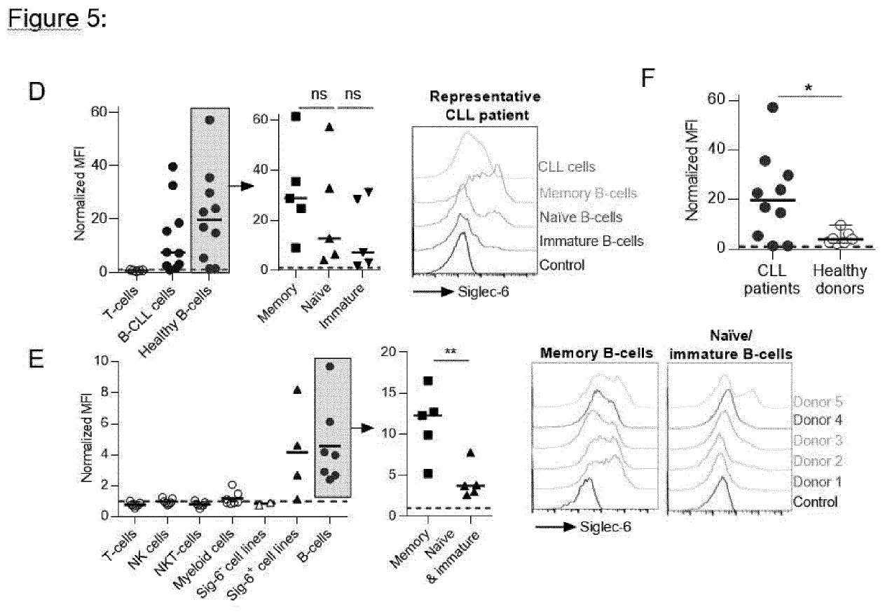

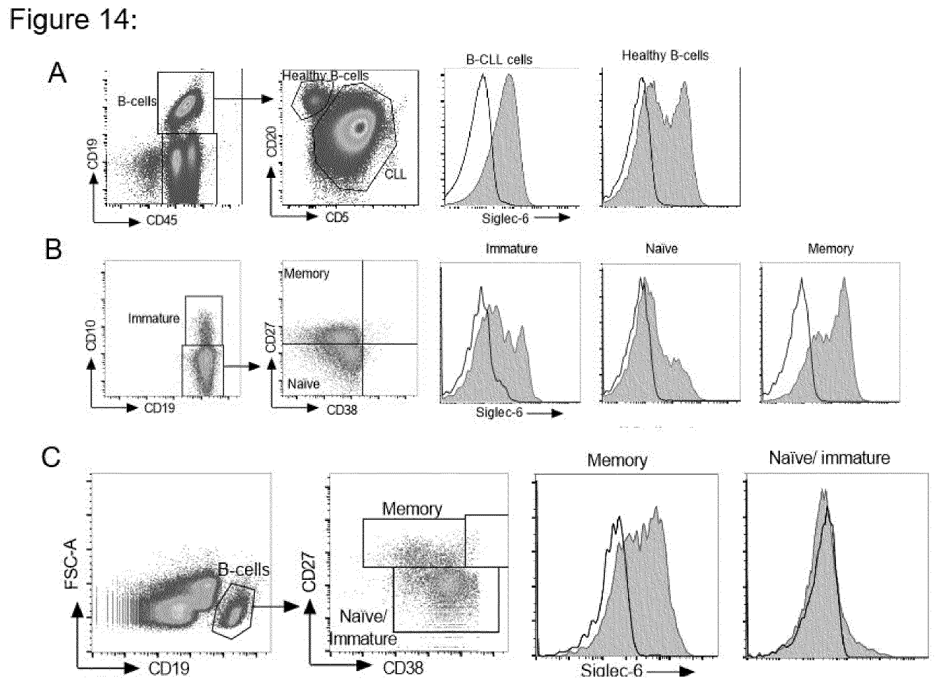

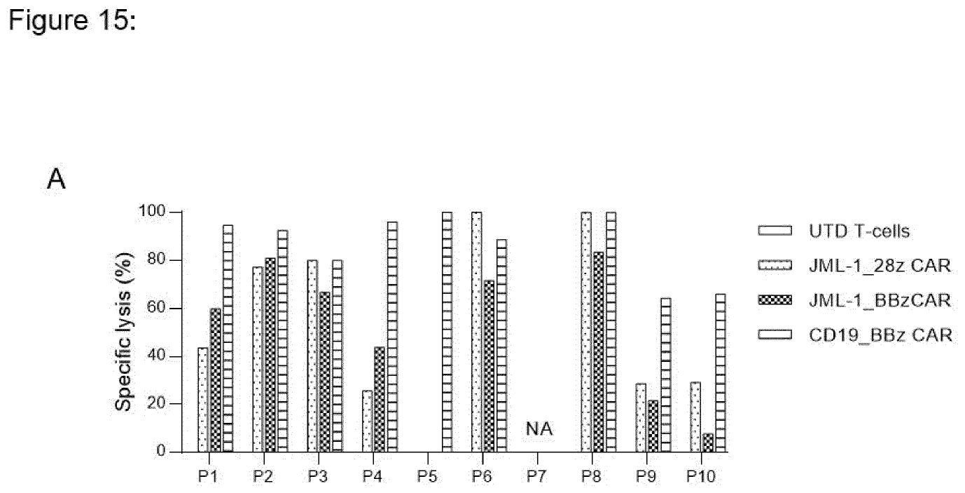

The inventors evaluated siglec-6-expression on PB derived primary B-CLL cells from treatment-naïve CLL patients (n=10, Table-2). The inventors show that siglec-6 is expressed uniformly and at high levels on primary B-CLL cells in 9 out of these 10 patients (FIG. 5A, Table-2, and FIG. 14A). To assess recognition of primary B-CLL cells, the inventors co-cultured PBMCs from patients with CD8+JML-1-CAR T-cells. The inventors observed high-levels of cytolytic activity by JML-1_28z and JML-1_BBz CAR T-cells against siglec-6-positive B-CLL cells within 4-hour of co-culture (FIG. 5B and Table-2). CLL patients with very high siglec-6-expression (patient #2, 6 and 8) showed near-complete elimination of B-CLL cells, comparable to lysis observed with CD19-CAR T-cells within the 4-hour assay period (FIG. 5B). Moreover, cytolytic activity of JML-1_BBz-CAR T-cells showed linear correlation to siglec-6-expression levels on B-CLL cells (FIG. 5C). The inventors also observed siglec-6-expression on non-CLL B-cells, particularly memory B-cells (FIG. 5D, FIG. 14B). Healthy B-cells (CD19⁺CD5⁻CD20^{high} non B-CLL cells) from B-CLL patients were recognized by JML-1-CAR T-cells, at levels that were similar to CD19-CAR T-cells (FIG. 15).

Taken together, these data show that siglec-6 is expressed on malignant B-cells in B-CLL and demonstrate that JML-1-CAR T-cells rapidly and potently eliminate malignant B-CLL cells.

Siglec-6 is Expressed on a Subset of Normal B-Cells and Confers Recognition by JML-1-CAR T-Cells

Next, the inventors sought to analyze siglec-6-expression on HD derived PBMCs (n=7). The inventors detected high-levels of siglec-6 on fraction of B-cells in flow analysis while other healthy PB cells i.e. NK cells, T-cells, NKT cells do not express siglec-6 (FIG. 5E). The inventors detected a small fraction of CD33+myeloid cells that express low levels of siglec-6 (FIG. 5E). The inventors observed significantly higher siglec-6-expression on memory B-cells when compared with naïve/immature B-cells in HD (FIG. 5E, FIG. 14C). Notably, each HD had memory B-cells with low to very high siglec-6-expression (Histogram, FIG. 5E). When the inventors compared siglec-6-expression levels on normal B-cells in CLL patients and HD, the inventors observed significantly lower siglec-6 levels in heathy donors compared to CLL patients (FIG. 5F). Therefore, the inventors anticipate siglec-6+memory and naïve B-cells to be susceptible for CAR-mediated recognition and elimination when patients with B-CLL are treated with JML-1-CAR T-cells.

Taken together, these data show that siglec-6 is expressed on a subset of normal B-cells in healthy donors and patients, suggesting that an anticipated on-target off-tumor effect of targeting siglec-6 will be selective, partial deletion of normal B-cells.

TABLE 2
Expression of siglec-6 in primary B-CLL and anti-B-CLL reactivity
of JML-1-CAR T-cells. Siglec-6 expression was analyzed in n =
10 newly diagnosed (previously untreated) B-CLL patients. NMFI:
The normalized mean fluorescence intensity (NMFI) is calculated
by dividing the MFI obtained by flow cytometry after staining with anti-
siglec-6 mAb through the MFI obtained by flow cytometry after staining
with an isotype control. The cytolytic activity of JML-1_BBz
CAR T-cells was analyzed in a 4-hour flow-cytometry-based cytolysis
assay, at an 5:1 effector to target cell ratio.

| | | | | CLL cells in | | Absolute |
|---|---|---|---|---|---|---|
| Patient | | IGHV | Cyto- | peripheral | | cell lysis |
| rank | Age | mutation | genetics | blood (%) | NMFI | (%) |
| 1 | 66 | No | +12 | 89 | 0.9 | 0 |
| 2 | 91 | Yes | — | 36 | 1.5 | 7 |
| 3 | 54 | Yes | del13q | 24 | 2.5 | 47 |
| 4 | 53 | Yes | — | 84 | 3.0 | 57 |
| 5 | 90 | Yes | del13q | 89 | 7.2 | 31 |
| 6 | 48 | No | del11q | 96 | 7.5 | 49 |
| 7 | 61 | Yes | — | 60 | 15.5 | 63 |
| 8 | 58 | Yes | — | 79 | 18.5 | 73 |
| 9 | 60 | Yes | — | 69 | 32.6 | 70 |
| 10 | 66 | Yes | +12 | 96 | 39.6 | 77 |

### DISCUSSION

The present inventors demonstrate that siglec-6 is a target for antibody-based and cellular immunotherapy in AML. In particular, the inventors demonstrate that siglec-6 is a target for antibody-based and cellular immunotherapy to target and destroy AML leukemic stem cells. The inventors demonstrate that JML-1-CAR T-cells confer potent anti-leukemia efficacy against primary AML blasts in vitro and induce complete remissions of leukemia in mice engrafted with AML cell lines.

Importantly, siglec-6 was found to be absent on normal HSC/P. Accordingly, JML-1-CAR T-cells did not recognize normal HSC/P and did not lead to a reduction in hematopoietic lineage development in colony formation experiments. The possibility of sparing normal HSC/P while eradicating AML blast and AML leukemic stem cells enables a non-myeloablative immunotherapy to effectively treat AML while eliminating the need for alloHSCT.

Siglec-6-expression on other healthy tissues is restricted to placenta [16], mast cells [17] and a subset of normal B-cells [10], suggesting a favorable safety profile with negligible on-target, off-tumor reactivity. The inventors found that the majority of memory B-cells but only a small proportion of naïve and immature B-cells express siglec-6, and therefore anticipate selective, partial deletion of normal B-cells to occur after anti-siglec-6 immunotherapy as e.g. JML-1 CAR-T cell therapy. Encouragingly, potential hypogammaglobulinemia can be overcome by intravenous immunoglobulin (IVIG) replacement therapy if needed, which is already common practice after CD19-CAR T-cell therapy [27]. Siglec-6 is reported to be present on mast cells [18] and therefore, mast cell reduction or depletion may occur after anti-siglec-6 immunotherapy.

Moreover, the inventors found high siglec-6 expression on primary B-CLL cells obtained from treatment-naïve CLL patients, which is in line with previous observations [11]. Indeed, the inventors observed potent anti-CLL activity of both JML-1_28z and JML-1_BBz-CAR T-cells against primary B-CLL cells in vitro. T-cells from CLL patients exhibit features of T-cell exhaustion and proliferative defects and therefore, patient-derived JML-1-CAR T-cells might not function equally well to that of HD-derived CAR T-cells [28, 23,24]. Encouragingly, a Bruton Tyrosine Kinase (BTK) inhibitor ibrutinib has shown to improve the anti-leukemia efficacy of CD19-CAR T-cells in mouse models and in CLL patients [28,29]. Therefore, the combination of ibrutinib could improve effectiveness of JML-1-CAR T-cell therapy in CLL patients and warrants preclinical evaluation for synergy of JML-1-CAR T-cell with ibrutinib.

CAR T-cell rejection due to transgene containing murine scFv (e.g. FMC63) is a mechanism that contributes to the resistance to CAR T-cell therapy [30]. JML-1-CAR is derived from a fully human scFv and therefore it is unlikely to be immunogenic. This enables the administration of multiple, sequential infusions of JML-1-CAR T-cells to further augment and to sustain the anti-leukemia response, and to avoid alloHSCT, if needed.

Moreover, leukemia relapse due to target antigen loss or downregulation has been observed in 30-60% of all relapses in B-ALL patients treated with CD19-CAR or CD22-CAR T-cells [9,31,32]. Although there is a uniform siglec-6-expression on AML blasts, due to clonal heterogeneity, there is a potential risk that siglec-6 expression may change under the therapeutic pressure from anti-siglec-6 immunotherapy. However, there is no prior clinical experience with siglec-6 as an immune target.

Furthermore, the possibility of protein truncation resulting in siglec-6 protein without transmembrane domain and therefore to a loss of cell surface siglec-6 cannot be eliminated, as reported with CD19 protein after CD19-CAR T-cell therapy in B-ALL patients [33]. Preclinical mouse models mimicking antigen loss escape could help to investigate antigen loss escape after CAR T-cell treatment and strategies to prevent resistance to CAR-T cell therapy. Alternatively, the use of tandem or compound CARs targeting two or more antigens simultaneously or sequentially may prevent antigen loss escape in AML.

The inventors' findings on siglec-6-expression and anti-leukemia activity by JML-1-CAR T-cells suggests that a therapy using siglec-6-binding immune cells, such as JML-1 CAR-T cells, would be applicable to AML and CLL patients and warrants clinical investigation in humans. Moreover, siglec-6-expression is also reported on MALT lymphoma [15], in clonal mast cell disease [34], and in thymoma, extending the application of therapy using siglec-6-binding immune cells, such as JML-1-CAR T-cells, to other hematologic and oncologic indications, and to other applications in medicine.

### INDUSTRIAL APPLICABILITY

The siglec-6-binding polypeptide, the nucleotide sequence encoding the siglec-6-binding polypeptide, the expression vector as well as the immune cell comprising the siglec-6-binding polypeptide according to the invention, can be industrially manufactured and sold as products for the methods and uses as described (e.g. for treating a cancer as defined herein), in accordance with known standards for the manufacture of pharmaceutical products. Accordingly, the present invention is industrially applicable.

Sequences
(GMCSF signal peptide)
SEQ ID NO: 1
MLLLVTSLLLCELPHPAFLLIP
(DNA sequence encoding SEQ ID NO: 1)
SEQ ID NO: 2
ATGTTGCTGCTGGTTACATCTCTGCTGCTGTGCGAGCTGCCCCATCCTGCC
(JML-1 heavy chain variable domain (VH))
SEQ ID NO: 3
KVQLLESGGGLVQPGRSLRLSCAASGFTFDDYGMHWVRQAPGKGLEWVSGISWNSGSIGYADSVKGRF
TISRDNSKNTLYLQMNSLRAEDTAVYYCARGGQTIDIWGQGTMVTVSS
(DNA sequence encoding SEQ ID NO: 3)
SEQ ID NO: 4
AAGGTGCAGCTGCTGGAATCTGGCGGAGGACTGGTTCAGCCTGGCAGAAGCCTGAGACTGTCTTGT
GCCGCCAGCGGCTTCACCTTCGACGATTATGGCATGCACTGGGTCCGACAGGCCCCTGGCAAAGGAC
TTGAATGGGTGTCCGGCATCAGCTGGAACAGCGGCTCTATCGGCTACGCCGATTCCGTGAAGGGCA
GATTCACCATCAGCCGGGACAACAGCAAGAACACCCTGTACCTGCAGATGAACAGCCTGAGAGCCG
AGGACACCGCCGTGTACTATTGTGCTAGAGGCGGCCAGACCATCGACATCTGGGGACAGGGAACCA
TGGTCACCGTTTCTAGC
(4(GS)x3 linker)
SEQ ID NO: 5
GGGGSGGGGSGGGGS
(DNA sequence encoding SEQ ID NO: 5)
SEQ ID NO: 6
GGAGGCGGAGGTTCTGGCGGCGGAGGAAGTGGTGGCGGAGGCTCT
(JML-1 light chain variable domain (VL))
SEQ ID NO: 7
DIQMTQSPSSLSASVGDRVTITCRASQSISSYLNWYQQKPGKAPKLLIYAASSLQSGVPSRFSGSGSGTDFT
LTISSLQPEDFATYYCQQSYSTPFTFGPGTKVDIK
(DNA sequence encoding SEQ ID NO: 7)
SEQ ID NO: 8
GATATCCAGATGACACAGAGCCCCAGCAGCCTGTCTGCCTCTGTGGGAGACAGAGTGACCATCACCT
GTAGAGCCAGCCAGAGCATCAGCAGCTACCTGAACTGGTATCAGCAAAAGCCCGGCAAGGCCCCTA
AACTGCTGATCTACGCTGCCTCCAGTCTGCAGAGCGGAGTGCCTAGCAGATTTTCTGGCTCTGGCAG
CGGCACCGACTTCACCCTGACCATATCTAGCCTGCAGCCTGAGGACTTCGCCACCTACTACTGCCAGC
AGAGCTACAGCACCCCTTTCACATTTGGCCCTGGCACCAAGGTGGACATCAAA
(IgG4 hinge domain)
SEQ ID NO: 9
ESKYGPPCPPCP
(DNA sequence encoding SEQ ID NO: 9)
SEQ ID NO: 10
GAGTCTAAGTACGGACCGCCTTGTCCTCCTTGTCCA
(IgG3 hinge)
SEQ ID NO: 11
ELKTPLGDTTHTCPRCPEPKSCDTPPPCPRCP
(DNA sequence encoding SEQ ID NO: 11)
SEQ ID NO: 12
GAGCTGAAAACCCCTCTGGGCGACACCACACACACATGCCCTAGATGTCCGGAACCCAAGAGCTGCG
ATACCCCCCCACCTTGCCCCAGATGCCCC
(CD28 transmembrane domain)
SEQ ID NO: 13
MFWVLVVVGGVLACYSLLVTVAFIIFWV
(DNA sequence encoding SEQ ID NO: 13)
SEQ ID NO: 14
ATGTTTTGGGTGCTGGTGGTCGTGGGCGGAGTGCTGGCCTGTTACAGCCTGCTCGTGACCGTGGCCT
TCATCATCTTTTGGGTC
(CD28 costimulatory domain)
SEQ ID NO: 15
RSKRSRGGHSDYMNMTPRRPGPTRKHYQPYAPPRDFAAYRS
(DNA sequence encoding SEQ ID NO: 15)
SEQ ID NO: 16
CGCAGCAAGCGGAGCAGAGGCGGCCACAGCGACTACATGAACATGACCCCTAGACGGCCTGGCCCC
ACCAGAAAGCACTACCAGCCCTACGCCCCTCCCCGGGACTTTGCCGCCTACAGAAGC
(4-1BB costimulatory domain)
SEQ ID NO: 17
KRGRKKLLYIFKQPFMRPVQTTQEEDGCSCRFPEEEEGGCEL
(DNA sequence encoding SEQ ID NO: 17)
SEQ ID NO: 18
AAGCGGGGCAGAAAGAAGCTGCTGTACATCTTTAAGCAGCCCTTCATGCGGCCCGTGCAGACCACCC
AGGAAGAGGACGGCTGCTCCTGCAGATTCCCCGAGGAAGAAGAAGGCGGCTGCGAGCTG
(CD3zeta signaling domain)
SEQ ID NO: 19
RVKFSRSADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
(DNA sequence encoding SEQ ID NO: 19)
SEQ ID NO: 20
CGGGTGAAGTTCAGCAGAAGCGCCGACGCCCCTGCCTACCAGCAGGGCCAGAATCAGCTGTACAAC
GAGCTGAACCTGGGCAGAAGGGAAGAGTACGACGTCCTGGATAAGCGGAGAGGCCGGGACCCTGA
GATGGGCGGCAAGCCTCGGCGGAAGAACCCCCAGGAAGGCCTGTATAACGAACTGCAGAAAGACA
AGATGGCCGAGGCCTACAGCGAGATCGGCATGAAGGGCGAGCGGAGGCGGGGCAAGGGCCACGA
CGGCCTGTATCAGGGCCTGTCCACCGCCACCAAGGATACCTACGACGCCCTGCACATGCAGGCCCTG
CCCCCAAGG
(T2A ribosomal skipping sequence)
SEQ ID NO: 21
LEGGGEGRGSLLTCGDVEENPGPR
(DNA sequence encoding SEQ ID NO: 21)
SEQ ID NO: 22
CTCGAGGGCGGCGGAGAGGGCAGAGGAAGTCTTCTAACATGCGGTGACGTGGAGGAGAATCCCGG
CCCTAGG
(EGFRt)
SEQ ID NO: 23
RKVCNGIGIGEFKDSLSINATNIKHFKNCTSISGDLHILPVAFRGDSFTHTPPLDPQELDILKTVKEITGFLLIQ
AWPENRTDLHAFENLEIIRGRTKQHGQFSLAVVSLNITSLGLRSLKEISDGDVIISGNKNLCYANTINWKKLF
GTSGQKTKIISNRGENSCKATGQVCHALCSPEGCWGPEPRDCVSCRNVSRGRECVDKCNLLEGEPREFVE
NSECIQCHPECLPQAMNITCTGRGPDNCIQCAHYIDGPHCVKTCPAGVMGENNTLVWKYADAGHVCH
LCHPNCTYGCTGPGLEGCPTNGPKIPSIATGMVGALLLLLVVALGIGLFM
(DNA sequence encoding SEQ ID NO: 23)
SEQ ID NO: 24
CGCAAAGTGTGTAACGGAATAGGTATTGGTGAATTTAAAGACTCACTCTCCATAAATGCTACGAATA
TTAAACACTTCAAAAACTGCACCTCCATCAGTGGCGATCTCCACATCCTGCCGGTGGCATTTAGGGGT
GACTCCTTCACACATACTCCTCCTCTGGATCCACAGGAACTGGATATTCTGAAAACCGTAAAGGAAAT
CACAGGGTTTTTGCTGATTCAGGCTTGGCCTGAAAACAGGACGGACCTCCATGCCTTTGAGAACCTA
GAAATCATACGCGGCAGGACCAAGCAACATGGTCAGTTTTCTCTTGCAGTCGTCAGCCTGAACATAA
CATCCTTGGGATTACGCTCCCTCAAGGAGATAAGTGATGGAGATGTGATAATTTCAGGAAACAAAAA
TTTGTGCTATGCAAATACAATAAACTGGAAAAAACTGTTTGGGACCTCCGGTCAGAAAACCAAAATT
ATAAGCAACAGAGGTGAAAACAGCTGCAAGGCCACAGGCCAGGTCTGCCATGCCTTGTGCTCCCCC
GAGGGCTGCTGGGGCCCGGAGCCCAGGGACTGCGTCTCTTGCCGGAATGTCAGCCGAGGCAGGGA
ATGCGTGGACAAGTGCAACCTTCTGGAGGGTGAGCCAAGGGAGTTTGTGGAGAACTCTGAGTGCAT
ACAGTGCCACCCAGAGTGCCTGCCTCAGGCCATGAACATCACCTGCACAGGACGGGGACCAGACAA
CTGTATCCAGTGTGCCCACTACATTGACGGCCCCCACTGCGTCAAGACCTGCCCGGCAGGAGTCATG
GGAGAAAACAACACCCTGGTCTGGAAGTACGCAGACGCCGGCCATGTGTGCCACCTGTGCCATCCA
AACTGCACCTACGGATGCACTGGGCCAGGTCTTGAAGGCTGTCCAACGAATGGGCCTAAGATCCCGT
CCATCGCCACTGGGATGGTGGGGGCCCTCCTCTTGCTGCTGGTGGTGGCCCTGGGGATCGGCCTCTT
CATGTGA
(JML-1 scFv)
SEQ ID NO: 25
KVQLLESGGGLVQPGRSLRLSCAASGFTFDDYGMHWVRQAPGKGLEWVSGISWNSGSIGYADSVKGRF
TISRDNSKNTLYLQMNSLRAEDTAVYYCARGGQTIDIWGQGTMVTVSSGGGGSGGGGSGGGGSDIQM
TQSPSSLSASVGDRVTITCRASQSISSYLNWYQQKPGKAPKLLIYAASSLQSGVPSRFSGSGSGTDFTLTISS
LQPEDFATYYCQQSYSTPFTFGPGTKVDIK
(DNA sequence encoding SEQ ID NO: 25)
SEQ ID NO: 26
AAGGTGCAGCTGCTGGAATCTGGCGGAGGACTGGTTCAGCCTGGCAGAAGCCTGAGACTGTCTTGT
GCCGCCAGCGGCTTCACCTTCGACGATTATGGCATGCACTGGGTCCGACAGGCCCCTGGCAAAGGAC
TTGAATGGGTGTCCGGCATCAGCTGGAACAGCGGCTCTATCGGCTACGCCGATTCCGTGAAGGGCA
GATTCACCATCAGCCGGGACAACAGCAAGAACACCCTGTACCTGCAGATGAACAGCCTGAGAGCCG
AGGACACCGCCGTGTACTATTGTGCTAGAGGCGGCCAGACCATCGACATCTGGGGACAGGGAACCA
TGGTCACCGTTTCTAGCGGAGGCGGAGGTTCTGGCGGCGGAGGAAGTGGTGGCGGAGGCTCTGAT
ATCCAGATGACACAGAGCCCCAGCAGCCTGTCTGCCTCTGTGGGAGACAGAGTGACCATCACCTGTA
GAGCCAGCCAGAGCATCAGCAGCTACCTGAACTGGTATCAGCAAAAGCCCGGCAAGGCCCCTAAAC
TGCTGATCTACGCTGCCTCCAGTCTGCAGAGCGGAGTGCCTAGCAGATTTTCTGGCTCTGGCAGCGG
CACCGACTTCACCCTGACCATATCTAGCCTGCAGCCTGAGGACTTCGCCACCTACTACTGCCAGCAGA
GCTACAGCACCCCTTTCACATTTGGCCCTGGCACCAAGGTGGACATCAAA
(full-length CAR with IgG4 hinge and CD28 costimulatory domain)
SEQ ID NO: 27
MLLLVTSLLLCELPHPAFLLIPKVQLLESGGGLVQPGRSLRLSCAASGFTFDDYGMHWVRQAPGKGLEWV
SGISWNSGSIGYADSVKGRFTISRDNSKNTLYLQMNSLRAEDTAVYYCARGGQTIDIWGQGTMVTVSSG
GGGSGGGGSGGGGSDIQMTQSPSSLSASVGDRVTITCRASQSISSYLNWYQQKPGKAPKLLIYAASSLQS
GVPSRFSGSGSGTDFTLTISSLQPEDFATYYCQQSYSTPFTFGPGTKVDIKESKYGPPCPPCPMFWVLVVV
GGVLACYSLLVTVAFIIFWVRSKRSRGGHSDYMNMTPRRPGPTRKHYQPYAPPRDFAAYRSRVKFSRSA
DAPAYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
(DNA sequence encoding SEQ ID NO: 27)
SEQ ID NO: 28
AAGGTGCAGCTGCTGGAATCTGGCGGAGGACTGGTTCAGCCTGGCAGAAGCCTGAGACTGTCTTGT
GCCGCCAGCGGCTTCACCTTCGACGATTATGGCATGCACTGGGTCCGACAGGCCCCTGGCAAAGGAC
TTGAATGGGTGTCCGGCATCAGCTGGAACAGCGGCTCTATCGGCTACGCCGATTCCGTGAAGGGCA
GATTCACCATCAGCCGGGACAACAGCAAGAACACCCTGTACCTGCAGATGAACAGCCTGAGAGCCG
AGGACACCGCCGTGTACTATTGTGCTAGAGGCGGCCAGACCATCGACATCTGGGGACAGGGAACCA
TGGTCACCGTTTCTAGCGGAGGCGGAGGTTCTGGCGGCGGAGGAAGTGGTGGCGGAGGCTCTGAT
ATCCAGATGACACAGAGCCCCAGCAGCCTGTCTGCCTCTGTGGGAGACAGAGTGACCATCACCTGTA
GAGCCAGCCAGAGCATCAGCAGCTACCTGAACTGGTATCAGCAAAAGCCCGGCAAGGCCCCTAAAC
TGCTGATCTACGCTGCCTCCAGTCTGCAGAGCGGAGTGCCTAGCAGATTTTCTGGCTCTGGCAGCGG
CACCGACTTCACCCTGACCATATCTAGCCTGCAGCCTGAGGACTTCGCCACCTACTACTGCCAGCAGA
GCTACAGCACCCCTTTCACATTTGGCCCTGGCACCAAGGTGGACATCAAA
(full-length CAR with IgG4 hinge and 4-1BB costimulatory domain)
SEQ ID NO: 29
MLLLVTSLLLCELPHPAFLLIPKVQLLESGGGLVQPGRSLRLSCAASGFTFDDYGMHWVRQAPGKGLEWV
SGISWNSGSIGYADSVKGRFTISRDNSKNTLYLQMNSLRAEDTAVYYCARGGQTIDIWGQGTMVTVSSG
GGGSGGGGSGGGGSDIQMTQSPSSLSASVGDRVTITCRASQSISSYLNWYQQKPGKAPKLLIYAASSLQS
GVPSRFSGSGSGTDFTLTISSLQPEDFATYYCQQSYSTPFTFGPGTKVDIKESKYGPPCPPCPMFWVLVVV
GGVLACYSLLVTVAFIIFWVKRGRKKLLYIFKQPFMRPVQTTQEEDGCSCRFPEEEEGGCELRVKFSRSADA
PAYQQGQNQLYNELNLGRREEYDVLDKRRGRDPEMGGKPRRKNPQEGLYNELQKDKMAEAYSEIGMK
GERRRGKGHDGLYQGLSTATKDTYDALHMQALPPR
(DNA sequence encoding SEQ ID NO: 29)
SEQ ID NO: 30
ATGTTGCTGCTGGTTACATCTCTGCTGCTGTGCGAGCTGCCCCATCCTGCCTTTCTGCTGATCCCTAAG
GTGCAGCTGCTGGAATCTGGCGGAGGACTGGTTCAGCCTGGCAGAAGCCTGAGACTGTCTTGTGCC
GCCAGCGGCTTCACCTTCGACGATTATGGCATGCACTGGGTCCGACAGGCCCCTGGCAAAGGACTTG
AATGGGTGTCCGGCATCAGCTGGAACAGCGGCTCTATCGGCTACGCCGATTCCGTGAAGGGCAGAT
TCACCATCAGCCGGGACAACAGCAAGAACACCCTGTACCTGCAGATGAACAGCCTGAGAGCCGAGG
ACACCGCCGTGTACTATTGTGCTAGAGGCGGCCAGACCATCGACATCTGGGGACAGGGAACCATGG
TCACCGTTTCTAGCGGAGGCGGAGGTTCTGGCGGCGGAGGAAGTGGTGGCGGAGGCTCTGATATCC
AGATGACACAGAGCCCCAGCAGCCTGTCTGCCTCTGTGGGAGACAGAGTGACCATCACCTGTAGAG
CCAGCCAGAGCATCAGCAGCTACCTGAACTGGTATCAGCAAAAGCCCGGCAAGGCCCCTAAACTGCT
GATCTACGCTGCCTCCAGTCTGCAGAGCGGAGTGCCTAGCAGATTTTCTGGCTCTGGCAGCGGCACC
GACTTCACCCTGACCATATCTAGCCTGCAGCCTGAGGACTTCGCCACCTACTACTGCCAGCAGAGCTA
CAGCACCCCTTTCACATTTGGCCCTGGCACCAAGGTGGACATCAAAGAGTCTAAGTACGGACCGCCC
TGCCCCCCTTGCCCTATGTTCTGGGTGCTGGTGGTGGTCGGAGGCGTGCTGGCCTGCTACAGCCTGC
TGGTCACCGTGGCCTTCATCATCTTTTGGGTGAAACGGGGCAGAAAGAAACTCCTGTATATATTCAA
ACAACCATTTATGAGACCAGTACAAACTACTCAAGAGGAAGATGGCTGTAGCTGCCGATTTCCAGAA
GAAGAAGAAGGAGGATGTGAACTGCGGGTGAAGTTCAGCAGAAGCGCCGACGCCCCTGCCTACCA
GCAGGGCCAGAATCAGCTGTACAACGAGCTGAACCTGGGCAGAAGGGAAGAGTACGACGTCCTGG
ATAAGCGGAGAGGCCGGGACCCTGAGATGGGCGGCAAGCCTCGGCGGAAGAACCCCCAGGAAGGC
CTGTATAACGAACTGCAGAAAGACAAGATGGCCGAGGCCTACAGCGAGATCGGCATGAAGGGCGA
GCGGAGGCGGGGCAAGGGCCACGACGGCCTGTATCAGGGCCTGTCCACCGCCACCAAGGATACCTA
CGACGCCCTGCACATGCAGGCCCTGCCCCCAAGG
(full-length CAR with IgG3 hinge and CD28 costimulatory domain)
SEQ ID NO: 31
MLLLVTSLLLCELPHPAFLLIPKVQLLESGGGLVQPGRSLRLSCAASGFTFDDYGMHWVRQAPGKGLEWV
SGISWNSGSIGYADSVKGRFTISRDNSKNTLYLQMNSLRAEDTAVYYCARGGQTIDIWGQGTMVTVSSG
GGGSGGGGSGGGGSDIQMTQSPSSLSASVGDRVTITCRASQSISSYLNWYQQKPGKAPKLLIYAASSLQS
GVPSRFSGSGSGTDFTLTISSLQPEDFATYYCQQSYSTPFTFGPGTKVDIKELKTPLGDTTHTCPRCPEPKSC
DTPPPCPRCPMFWVLVVVGGVLACYSLLVTVAFIIFWVRSKRSRGGHSDYMNMTPRRPGPTRKHYQPY
APPRDFAAYRSRVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
(DNA sequence encoding SEQ ID NO: 31)
SEQ ID NO: 32
ATGTTGCTGCTGGTTACATCTCTGCTGCTGTGCGAGCTGCCCCATCCTGCCTTTCTGCTGATCCCTAAG
GTGCAGCTGCTGGAATCTGGCGGAGGACTGGTTCAGCCTGGCAGAAGCCTGAGACTGTCTTGTGCC
GCCAGCGGCTTCACCTTCGACGATTATGGCATGCACTGGGTCCGACAGGCCCCTGGCAAAGGACTTG
AATGGGTGTCCGGCATCAGCTGGAACAGCGGCTCTATCGGCTACGCCGATTCCGTGAAGGGCAGAT
TCACCATCAGCCGGGACAACAGCAAGAACACCCTGTACCTGCAGATGAACAGCCTGAGAGCCGAGG
ACACCGCCGTGTACTATTGTGCTAGAGGCGGCCAGACCATCGACATCTGGGGACAGGGAACCATGG
TCACCGTTTCTAGCGGAGGCGGAGGTTCTGGCGGCGGAGGAAGTGGTGGCGGAGGCTCTGATATCC
AGATGACACAGAGCCCCAGCAGCCTGTCTGCCTCTGTGGGAGACAGAGTGACCATCACCTGTAGAG
CCAGCCAGAGCATCAGCAGCTACCTGAACTGGTATCAGCAAAAGCCCGGCAAGGCCCCTAAACTGCT
GATCTACGCTGCCTCCAGTCTGCAGAGCGGAGTGCCTAGCAGATTTTCTGGCTCTGGCAGCGGCACC
GACTTCACCCTGACCATATCTAGCCTGCAGCCTGAGGACTTCGCCACCTACTACTGCCAGCAGAGCTA
CAGCACCCCTTTCACATTTGGCCCTGGCACCAAGGTGGACATCAAAGAGCTGAAAACCCCTCTGGGC
GACACCACACACACATGCCCTAGATGTCCGGAACCCAAGAGCTGCGATACCCCCCCACCTTGCCCCA
GATGCCCCATGTTTTGGGTGCTGGTGGTCGTGGGCGGAGTGCTGGCCTGTTACAGCCTGCTCGTGAC
CGTGGCCTTCATCATCTTTTGGGTCCGCAGCAAGCGGAGCAGAGGCGGCCACAGCGACTACATGAA
CATGACCCCTAGACGGCCTGGCCCCACCAGAAAGCACTACCAGCCCTACGCCCCTCCCCGGGACTTT
GCCGCCTACAGAAGCCGGGTGAAGTTCAGCAGAAGCGCCGACGCCCCTGCCTACCAGCAGGGCCAG
AATCAGCTGTACAACGAGCTGAACCTGGGCAGAAGGGAAGAGTACGACGTCCTGGATAAGCGGAG
AGGCCGGGACCCTGAGATGGGCGGCAAGCCTCGGCGGAAGAACCCCCAGGAAGGCCTGTATAACG
AACTGCAGAAAGACAAGATGGCCGAGGCCTACAGCGAGATCGGCATGAAGGGCGAGCGGAGGCG
GGGCAAGGGCCACGACGGCCTGTATCAGGGCCTGTCCACCGCCACCAAGGATACCTACGACGCCCT
GCACATGCAGGCCCTGCCCCCAAGG
(full-length CAR with IgG3 hinge and 4-1BB costimulatory domain)
SEQ ID NO: 33
MLLLVTSLLLCELPHPAFLLIPKVQLLESGGGLVQPGRSLRLSCAASGFTFDDYGMHWVRQAPGKGLEWV
SGISWNSGSIGYADSVKGRFTISRDNSKNTLYLQMNSLRAEDTAVYYCARGGQTIDIWGQGTMVTVSSG
GGGSGGGGSGGGGSDIQMTQSPSSLSASVGDRVTITCRASQSISSYLNWYQQKPGKAPKLLIYAASSLQS
GVPSRFSGSGSGTDFTLTISSLQPEDFATYYCQQSYSTPFTFGPGTKVDIKELKTPLGDTTHTCPRCPEPKSC
DTPPPCPRCPMFWVLVVVGGVLACYSLLVTVAFIIFWVKRGRKKLLYIFKQPFMRPVQTTQEEDGCSCRF
PEEEEGGCELRVKFSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR
(DNA sequence encoding SEQ ID NO: 33)
SEQ ID NO: 34
ATGTTGCTGCTGGTTACATCTCTGCTGCTGTGCGAGCTGCCCCATCCTGCCTTTCTGCTGATCCCTAAG
GTGCAGCTGCTGGAATCTGGCGGAGGACTGGTTCAGCCTGGCAGAAGCCTGAGACTGTCTTGTGCC
GCCAGCGGCTTCACCTTCGACGATTATGGCATGCACTGGGTCCGACAGGCCCCTGGCAAAGGACTTG
AATGGGTGTCCGGCATCAGCTGGAACAGCGGCTCTATCGGCTACGCCGATTCCGTGAAGGGCAGAT
TCACCATCAGCCGGGACAACAGCAAGAACACCCTGTACCTGCAGATGAACAGCCTGAGAGCCGAGG
ACACCGCCGTGTACTATTGTGCTAGAGGCGGCCAGACCATCGACATCTGGGGACAGGGAACCATGG
TCACCGTTTCTAGCGGAGGCGGAGGTTCTGGCGGCGGAGGAAGTGGTGGCGGAGGCTCTGATATCC
AGATGACACAGAGCCCCAGCAGCCTGTCTGCCTCTGTGGGAGACAGAGTGACCATCACCTGTAGAG
CCAGCCAGAGCATCAGCAGCTACCTGAACTGGTATCAGCAAAAGCCCGGCAAGGCCCCTAAACTGCT
GATCTACGCTGCCTCCAGTCTGCAGAGCGGAGTGCCTAGCAGATTTTCTGGCTCTGGCAGCGGCACC
GACTTCACCCTGACCATATCTAGCCTGCAGCCTGAGGACTTCGCCACCTACTACTGCCAGCAGAGCTA
CAGCACCCCTTTCACATTTGGCCCTGGCACCAAGGTGGACATCAAAGAGCTGAAAACCCCTCTGGGC
GACACCACACACACATGCCCTAGATGTCCGGAACCCAAGAGCTGCGATACCCCCCCACCTTGCCCCA
GATGCCCCATGTTTTGGGTGCTGGTGGTCGTGGGCGGAGTGCTGGCCTGTTACAGCCTGCTCGTGAC
CGTGGCCTTCATCATCTTTTGGGTCAAGCGGGGCAGAAAGAAGCTGCTGTACATCTTTAAGCAGCCC
TTCATGCGGCCCGTGCAGACCACCCAGGAAGAGGACGGCTGCTCCTGCAGATTCCCCGAGGAAGAA
GAAGGCGGCTGCGAGCTGAGAGTGAAGTTCAGCAGATCCGCCGACGCCCCTGCCTATCAGCAGGGC
CAGAACCAGCTATACAACGAGCTGAACCTGGGCAGACGGGAAGAGTACGACGTGCTGGACAAGAG
AAGAGGCCGGGACCCTGAGATGGGCGGAAAGCCCAGAAGAAAGAACCCCCAGGAAGGCCTGTATA
ACGAACTGCAGAAAGACAAGATGGCCGAGGCCTACAGCGAGATCGGAATGAAGGGCGAGCGGCG
GAGAGGCAAGGGCCACGATGGACTGTATCAGGGCCTGAGCACCGCCACCAAGGACACCTATGACGC
CCTGCACATGCAGGCCCTGCCCCCTAGA
(extracellular domain with IgG4 hinge)
SEQ ID NO: 35
MLLLVTSLLLCELPHPAFLLIPKVQLLESGGGLVQPGRSLRLSCAASGFTFDDYGMHWVRQAPGKGLEWV
SGISWNSGSIGYADSVKGRFTISRDNSKNTLYLQMNSLRAEDTAVYYCARGGQTIDIWGQGTMVTVSSG
GGGSGGGGSGGGGSDIQMTQSPSSLSASVGDRVTITCRASQSISSYLNWYQQKPGKAPKLLIYAASSLQS
GVPSRFSGSGSGTDFTLTISSLOPEDFATYYCQQSYSTPFTFGPGTKVDIKESKYGPPCPPCP
(DNA sequence encoding SEQ ID NO: 35)
SEQ ID NO: 36
ATGTTGCTGCTGGTTACATCTCTGCTGCTGTGCGAGCTGCCCCATCCTGCCTTTCTGCTGATCCCTAAG
GTGCAGCTGCTGGAATCTGGCGGAGGACTGGTTCAGCCTGGCAGAAGCCTGAGACTGTCTTGTGCC
GCCAGCGGCTTCACCTTCGACGATTATGGCATGCACTGGGTCCGACAGGCCCCTGGCAAAGGACTTG
AATGGGTGTCCGGCATCAGCTGGAACAGCGGCTCTATCGGCTACGCCGATTCCGTGAAGGGCAGAT
TCACCATCAGCCGGGACAACAGCAAGAACACCCTGTACCTGCAGATGAACAGCCTGAGAGCCGAGG
ACACCGCCGTGTACTATTGTGCTAGAGGCGGCCAGACCATCGACATCTGGGGACAGGGAACCATGG
TCACCGTTTCTAGCGGAGGCGGAGGTTCTGGCGGCGGAGGAAGTGGTGGCGGAGGCTCTGATATCC
AGATGACACAGAGCCCCAGCAGCCTGTCTGCCTCTGTGGGAGACAGAGTGACCATCACCTGTAGAG
CCAGCCAGAGCATCAGCAGCTACCTGAACTGGTATCAGCAAAAGCCCGGCAAGGCCCCTAAACTGCT
GATCTACGCTGCCTCCAGTCTGCAGAGCGGAGTGCCTAGCAGATTTTCTGGCTCTGGCAGCGGCACC
GACTTCACCCTGACCATATCTAGCCTGCAGCCTGAGGACTTCGCCACCTACTACTGCCAGCAGAGCTA
CAGCACCCCTTTCACATTTGGCCCTGGCACCAAGGTGGACATCAAAGAGTCTAAGTACGGACCGCCC
TGCCCCCCTTGCCCT
(extracellular domain with IgG3 hinge)
SEQ ID NO: 37
MLLLVTSLLLCELPHPAFLLIPKVQLLESGGGLVQPGRSLRLSCAASGFTFDDYGMHWVRQAPGKGLEWV
SGISWNSGSIGYADSVKGRFTISRDNSKNTLYLQMNSLRAEDTAVYYCARGGQTIDIWGQGTMVTVSSG
GGGSGGGGSGGGGSDIQMTQSPSSLSASVGDRVTITCRASQSISSYLNWYQQKPGKAPKLLIYAASSLQS
GVPSRFSGSGSGTDFTLTISSLQPEDFATYYCQQSYSTPFTFGPGTKVDIKELKTPLGDTTHTCPRCPEPKSC
DTPPPCPRCP
(DNA sequence encoding SEQ ID NO: 37)
SEQ ID NO: 38
ATGTTGCTGCTGGTTACATCTCTGCTGCTGTGCGAGCTGCCCCATCCTGCCTTTCTGCTGATCCCTAAG
GTGCAGCTGCTGGAATCTGGCGGAGGACTGGTTCAGCCTGGCAGAAGCCTGAGACTGTCTTGTGCC
GCCAGCGGCTTCACCTTCGACGATTATGGCATGCACTGGGTCCGACAGGCCCCTGGCAAAGGACTTG
AATGGGTGTCCGGCATCAGCTGGAACAGCGGCTCTATCGGCTACGCCGATTCCGTGAAGGGCAGAT
TCACCATCAGCCGGGACAACAGCAAGAACACCCTGTACCTGCAGATGAACAGCCTGAGAGCCGAGG
ACACCGCCGTGTACTATTGTGCTAGAGGCGGCCAGACCATCGACATCTGGGGACAGGGAACCATGG
TCACCGTTTCTAGCGGAGGCGGAGGTTCTGGCGGCGGAGGAAGTGGTGGCGGAGGCTCTGATATCC
AGATGACACAGAGCCCCAGCAGCCTGTCTGCCTCTGTGGGAGACAGAGTGACCATCACCTGTAGAG
CCAGCCAGAGCATCAGCAGCTACCTGAACTGGTATCAGCAAAAGCCCGGCAAGGCCCCTAAACTGCT
GATCTACGCTGCCTCCAGTCTGCAGAGCGGAGTGCCTAGCAGATTTTCTGGCTCTGGCAGCGGCACC
GACTTCACCCTGACCATATCTAGCCTGCAGCCTGAGGACTTCGCCACCTACTACTGCCAGCAGAGCTA
CAGCACCCCTTTCACATTTGGCCCTGGCACCAAGGTGGACATCAAAGAGCTGAAAACCCCTCTGGGC
GACACCACACACACATGCCCTAGATGTCCGGAACCCAAGAGCTGCGATACCCCCCCACCTTGCCCCA
GATGCCCC
(intracellular domain with CD28 costimulatory domain)
SEQ ID NO: 39
RSKRSRGGHSDYMNMTPRRPGPTRKHYQPYAPPRDFAAYRSRVKFSRSADAPAYQQGQNQLYNELNL
GRREEYDVLDKRRGRDPEMGGKPRRKNPQEGLYNELQKDKMAEAYSEIGMKGERRRGKGHDGLYQGL
STATKDTYDALHMQALPPR
(DNA sequence encoding SEQ ID NO: 39)
SEQ ID NO: 40
CGCAGCAAGCGGAGCAGAGGCGGCCACAGCGACTACATGAACATGACCCCTAGACGGCCTGGCCCC
ACCAGAAAGCACTACCAGCCCTACGCCCCTCCCCGGGACTTTGCCGCCTACAGAAGCCGGGTGAAGT
TCAGCAGAAGCGCCGACGCCCCTGCCTACCAGCAGGGCCAGAATCAGCTGTACAACGAGCTGAACC
TGGGCAGAAGGGAAGAGTACGACGTCCTGGATAAGCGGAGAGGCCGGGACCCTGAGATGGGCGG
CAAGCCTCGGCGGAAGAACCCCCAGGAAGGCCTGTATAACGAACTGCAGAAAGACAAGATGGCCG
AGGCCTACAGCGAGATCGGCATGAAGGGCGAGCGGAGGCGGGGCAAGGGCCACGACGGCCTGTAT
CAGGGCCTGTCCACCGCCACCAAGGATACCTACGACGCCCTGCACATGCAGGCCCTGCCCCCAAGG
(intracellular domain with 4-1BB costimulatory domain)
SEQ ID NO: 41
KRGRKKLLYIFKQPFMRPVQTTQEEDGCSCRFPEEEEGGCELRVKFSRSADAPAYQQGQNQLYNELNLGR
REEYDVLDKRRGRDPEMGGKPRRKNPQEGLYNELQKDKMAEAYSEIGMKGERRRGKGHDGLYQGLST
ATKDTYDALHMQALPPR
(DNA sequence encoding SEQ ID NO: 41)
SEQ ID NO: 42
AAACGGGGCAGAAAGAAACTCCTGTATATATTCAAACAACCATTTATGAGACCAGTACAAACTACTC
AAGAGGAAGATGGCTGTAGCTGCCGATTTCCAGAAGAAGAAGAAGGAGGATGTGAACTGCGGGTG
AAGTTCAGCAGAAGCGCCGACGCCCCTGCCTACCAGCAGGGCCAGAATCAGCTGTACAACGAGCTG
AACCTGGGCAGAAGGGAAGAGTACGACGTCCTGGATAAGCGGAGAGGCCGGGACCCTGAGATGGG
CGGCAAGCCTCGGCGGAAGAACCCCCAGGAAGGCCTGTATAACGAACTGCAGAAAGACAAGATGG
CCGAGGCCTACAGCGAGATCGGCATGAAGGGCGAGCGGAGGCGGGGCAAGGGCCACGACGGCCT
GTATCAGGGCCTGTCCACCGCCACCAAGGATACCTACGACGCCCTGCACATGCAGGCCCTGCCCCCA
AGG
(left IR/DR segment)
SEQ ID NO: 43
CAGTTGAAGTCGGAAGTTTACATACACTTAAGTTGGAGTCATTAAAACTCGTTTTTCAACTACTCCAC
AAATTTCTTGTTAACAAACAATAGTTTTGGCAAGTCAGTTAGGACATCTACTTTGTGCATGACACAAG
TCATTTTTCCAACAATTGTTTACAGACAGATTATTTCACTTATAATTCACTGTATCACAATTCCAGTGG
GTCAGAAGTTTACATACACT
(right IR/DR segment)
SEQ ID NO: 44
AGTGTATGTAAACTTCTGACCCACTGGGAATGTGATGAAAGAAATAAAAGCTGAAATGAATCATTCT
CTCTACTATTATTCTGATATTTCACATTCTTAAAATAAAGTGGTGATCCTAACTGACCTAAGACAGGGA
ATTTTTACTAGGATTAAATGTCAGGAATTGTGAAAAAGTGAGTTTAAATGTATTTGGCTAAGGTGTAT
GTAAACTTCCGACTTCAACTG
(Sleeping Beauty amino acid sequence)
SEQ ID NO: 45
MGKSKEISQDLRKRIVDLHKSGSSLGAISKRLAVPRSSVQTIVRKYKHHGTTQPSYRSGRRRVLSPRDERTL
VRKVQINPRTTAKDLVKMLEETGTKVSISTVKRVLYRHNLKGHSARKKPLLQNRHKKARLRFATAHGDKD
RTFWRNVLWSDETKIELFGHNDHRYVWRKKGEACKPKNTIPTVKHGGGSIMLWGCFAAGGTGALHKID
GIMDAVQYVDILKQHLKTSVRKLKLGRKWVFQHDNDPKHTSKVVAKWLKDNKVKVLEWPSQSPDLNPI
ENLWAELKKRVRARRPTNLTQLHQLCQEEWAKIHPNYCGKLVEGYPKRLTQVKQFKGNATKY
(DNA sequence encoding full-length siglec-6)
SEQ ID NO: 46
ATGCAGGGAGCCCAGGAAGCCTCCGCCTCAGAGATGCTACCGCTGCTGCTGCCCCTGCTGTGGGCA
GGGGCCCTGGCTCAGGAGCGGAGATTCCAGCTGGAGGGGCCAGAGTCACTGACGGTGCAGGAGGG
TCTGTGCGTCCTCGTACCCTGCAGATTGCCCACTACCCTTCCAGCCTCGTACTATGGTTATGGCTACTG
GTTCCTGGAAGGGGCTGATGTTCCAGTGGCCACAAACGACCCAGACGAAGAAGTGCAGGAGGAGA
CCCGGGGCCGATTCCACCTCCTCTGGGATCCCAGAAGGAAGAACTGCTCCCTGAGCATCAGAGATGC
CCGGAGGAGGGACAATGCTGCATACTTCTTTCGGTTGAAGTCCAAATGGATGAAATACGGTTATACA
TCTTCCAAGCTCTCTGTGCGTGTGATGGCCCTGACCCACAGGCCCAACATCTCCATCCCAGGGACCCT
GGAGTCTGGCCATCCCAGCAATCTGACCTGCTCTGTGCCCTGGGTCTGTGAGCAGGGGACGCCCCCC
ATCTTCTCCTGGATGTCAGCTGCCCCCACCTCCCTGGGCCCCAGGACCACCCAGTCCTCGGTGCTCAC
AATCACCCCACGGCCCCAGGACCACAGCACCAACCTCACCTGTCAGGTGACGTTCCCTGGAGCCGGT
GTGACCATGGAGAGAACCATCCAGCTCAATGTCTCCTATGCTCCACAGAAAGTGGCCATCAGCATCTT
CCAAGGAAACAGCGCAGCCTTCAAAATCCTGCAAAACACCTCGTCCCTCCCTGTCCTGGAGGGCCAG
GCTCTGCGGCTGCTCTGTGATGCTGACGGCAACCCCCCTGCACACCTGAGCTGGTTCCAGGGCTTCCC
CGCCCTGAACGCCACCCCCATCTCCAATACCGGGGTCCTGGAGCTGCCTCAAGTAGGGTCTGCAGAA
GAAGGAGATTTCACCTGCCGTGCTCAGCATCCTCTGGGCTCCCTGCAAATCTCTCTGAGTCTCTTTGT
GCATTGGAAACCAGAAGGCAGGGCTGGTGGTGTCCTGGGAGCAGTCTGGGGAGCTAGCATCACAA
CCCTGGTTTTCCTCTGTGTTTGCTTCATCTTCAGAGTGAAGACTAGAAGGAAGAAAGCAGCCCAGCCA
GTGCAAAACACGGATGATGTGAACCCCGTCATGGTCTCAGGCTCCAGGGGTCATCAGCACCAGTTCC
AGACAGGCATAGTTTCAGACCACCCTGCTGAGGCTGGCCCCATCTCAGAAGATGAGCAGGAGCTCCA
CTACGCTGTCCTACACTTCCACAAGGTGCAACCTCAGGAACCAAAGGTCACCGACACTGAGTACTCA
GAAATCAAGATACACAAG

### REFERENCES

1. Neelapu S S, Locke F L, Bartlett N L, et al. Axicabtagene Ciloleucel CAR T-Cell Therapy in Refractory Large B-Cell Lymphoma. New England Journal of Medicine. 2017; 377(26):2531-2544.
2. Park J H, Riviere I, Gonen M, et al. Long-Term Follow-up of CD19 CAR Therapy in Acute Lymphoblastic Leukemia. New England Journal of Medicine. 2018; 378(5):449-459.
3. Schuster S J, Bishop M R, Tam C S, et al. Tisagenlecleucel in Adult Relapsed or Refractory Diffuse Large B-Cell Lymphoma. New England Journal of Medicine. 2018; 380(1):45-56.
4. Raje N, Berdeja J, Lin Y, et al. Anti-BCMA CAR T-Cell Therapy bb2121 in Relapsed or Refractory Multiple Myeloma. New England Journal of Medicine. 2019; 380(18):1726-1737.
5. Hofmann S, Schubert M-L, Wang L, et al. Chimeric antigen receptor (CAR) T cell therapy in acute myeloid leukemia (AML). Journal of clinical medicine. 2019;8(2):200.
6. Gill S. Chimeric antigen receptor T cell therapy in AML: How close are we? Best Practice & Research Clinical Haematology. 2016; 29(4):329-333.
7. Sauer T, Rooney C M. Current challenges for CAR T-cell therapy of acute myeloid leukemia. Transfusion. 2019;59(4):1171.
8. Von Gunten S, Bochner B S. Basic and clinical immunology of Siglecs. Annals of the New York Academy of Sciences. 2008; 1143:61.
9. Fry T J, Shah N N, Orentas R J, et al. CD22-targeted CAR T cells induce remission in B-ALL that is naïve or resistant to CD19-targeted CAR immunotherapy. Nature medicine. 2018;24(1):20.
10. Baskar S, Suschak J M, Samija I, et al. A human monoclonal antibody drug and target discovery platform for B-cell chronic lymphocytic leukemia based on allogeneic hematopoietic stem cell transplantation and phage display. Blood, The Journal of the American Society of Hematology. 2009; 114(20):4494-4502.
11. Chang J, Peng H, Shaffer B C, et al. Siglec-6 on chronic lymphocytic leukemia cells is a target for post-allogeneic hematopoietic stem cell transplantation antibodies. Cancer immunology research. 2018:canimm. 0102.2018.
12. Patel N, Brinkman-Van der Linden E C, Altmann S W, et al. OB-BP1/Siglec-6 a leptin- and sialic acid-binding protein of the immunoglobulin superfamily. Journal of Biological Chemistry. 1999; 274(32):22729-22738.
13. Nguyen D H, Ball E D, Varki A. Myeloid precursors and acute myeloid leukemia cells express multiple CD33-related Siglecs. Experimental hematology. 2006; 34(6):728-735.
14. Crocker P R, Varki A. Siglecs in the immune system. Immunology. 2001;103(2):137.
15. Chng W J, Remstein E D, Fonseca R, et al. Gene expression profiling of pulmonary mucosa-associated lymphoid tissue lymphoma identifies new biologic insights with potential diagnostic and therapeutic applications. Blood, The Journal of the American Society of Hematology. 2009; 113(3):635-645.
16. Brinkman-Van der Linden EC, Hurtado-Ziola N, Hayakawa T, et al. Human-specific expression of Siglec-6 in the placenta. Glycobiology. 2007; 17(9):922-931.
17. Yokoi H, Myers A, Matsumoto K, Crocker P, Saito H, Bochner B. Alteration and acquisition of Siglecs during in vitro maturation of CD34+progenitors into human mast cells. Allergy. 2006; 61(6):769-776.
18. Yu Y, Blokhuis B R, Diks M A, Keshavarzian A, Garssen J, Redegeld F A. Functional inhibitory Siglec-6 is upregulated in human colorectal cancer-associated mast cells.

Frontiers in immunology. 2018; 9:2138.
19. Gill S, Tasian SK, Ruella M, et al. Preclinical targeting of human acute myeloid leukemia and myeloablation using chimeric antigen receptor-modified T cells. Blood, The Journal of the American Society of Hematology. 2014; 123(15):2343-2354.
20. Haubner S, Perna F, Köhnke T, et al. Coexpression profile of leukemic stem cell markers for combinatorial targeted therapy in AML. Leukemia. 2019; 33(1):64-74.
21. Cellectis. Cellectis reports clinical hold of UCART123 studies; 2017.
22. Kim MY, Yu K-R, Kenderian SS, et al. Genetic inactivation of CD33 in hematopoietic stem cells to enable CAR T cell immunotherapy for acute myeloid leukemia. Cell. 2018; 173(6):1439-1453. e1419.
23. Jetani H, Garcia-Cadenas I, Nerreter T, et al. CAR T-cells targeting FLT3 have potent activity against FLT3− ITD+ AML and act synergistically with the FLT3-inhibitor crenolanib. Leukemia. 2018; 32(5):1168-1179.
24. Tashiro H, Sauer T, Shum T, et al. Treatment of acute myeloid leukemia with T cells expressing chimeric antigen receptors directed to C-type lectin-like molecule 1.

Molecular Therapy. 2017; 25(9):2202-2213.
25. Casucci M, Nicolis di Robilant B, Falcone L, et al. CD44v6-targeted T cells mediate potent antitumor effects against acute myeloid leukemia and multiple myeloma. Blood. 2013; 122(20):3461-3472.
26. Gomes-Silva D, Srinivasan M, Sharma S, et al. CD7-edited T cells expressing a CD7− specific CAR for the therapy of T-cell malignancies. Blood, The Journal of the American Society ofHematology. 2017; 130(3):285-296.
27. Bhoj VG, Arhontoulis D, Wertheim G, et al. Persistence of long-lived plasma cells and humoral immunity in individuals responding to CD19-directed CAR T-cell therapy. Blood. 2016; 128(3):360-370.
28. Fraietta JA, Beckwith KA, Patel PR, et al. Ibrutinib enhances chimeric antigen receptor T-cell engraftment and efficacy in leukemia. Blood. 2016; 127(9):1117-1127.
29. Gauthier J, Hirayama AV, Purushe J, et al. Feasibility and efficacy of CD19-targeted CAR T cells with concurrent ibrutinib for CLL after ibrutinib failure. Blood, The Journal of the American Society of Hematology. 2020; 135(19):1650-1660.
30. Jensen MC, Popplewell L, Cooper U, et al. Antitransgene rejection responses contribute to attenuated persistence of adoptively transferred CD20/CD19-specific chimeric antigen receptor redirected T cells in humans. Biology of blood and marrow transplantation. 2010; 16(9):1245-1256.
31. Maude SL, Frey N, Shaw PA, et al. Chimeric antigen receptor T cells for sustained remissions in leukemia. New England Journal of Medicine. 2014; 371(16):1507-1517.
32. Ruella M, Barrett DM, Kenderian SS, et al. Dual CD19 and CD123 targeting prevents antigen-loss relapses after CD19-directed immunotherapies. The Journal of clinical investigation. 2016; 126(10):3814-3826.
33. Orlando EJ, Han X, Tribouley C, et al. Genetic mechanisms of target antigen loss in CAR19 therapy of acute lymphoblastic leukemia. Nature medicine. 2018; 24(10):1504-1506.
34. Teufelberger AR WJ, Catherine OS et al. Siglec-6 a potential new biomarker for clonal mast cell diseases.
35. Majeti R, Weissman I. Human Acute Myelogenous Leukemia Stem Cells Revisited—There's More Than Meets the Eye. Cancer cell. 2011; 19(1):9-10
36. Haubner S, Perna F, K6hnke T et al. Coexpression profile of leukemic stem cell markers for combinatorial targeted therapy in AML. Leukemia. 2019; 33:64-74
37. Ivanivska T, Sklyarenko L, Zavelevich M et al. Immunophenotypic features of leukemic stem cells and bulk of blasts in acute myeloid leukemia. Experimental oncology. 2019; 41(3):207-209
38. Agarwal S, Weidner T, Thalheimer F, Buchholz C. In vivo generated human CAR T cells eradicate tumor cells. Oncoimmunology. 2019;8(12):e1671761.
39. Agarwal S, HanauerJDS, Frank AM, Riechert V, Thalheimer FB, Buchholz CJ. In vivo generation of CAR T cells selectively in human CD4+lymphocytes. Mol Ther. 28(8):1783-1794.
40. Smith TT, Stephan SB, Moffett HF, McKnight LE, Ji W, Reiman D, Bonagofski E, Wohlfahrt ME, Pillai SPS, Stephan MT. In situ programming of leukaemia-specific T cells using synthetic DNA nanocarriers. Nat Nanotechnol. 2017 August; 12(8):813-820.

1-80. (canceled)

81. A siglec-6-binding polypeptide that comprises or consists of a chimeric antigen receptor (CAR), the siglec-6-binding polypeptide comprising or consisting of a siglec-6-binding CAR, wherein the CAR comprises at least one extracellular ligand binding domain, a transmembrane domain and at least one intracellular signalling domain, wherein:
(i) said extracellular ligand binding domain comprises a siglec-6-binding element represented by an amino acid sequence shown in SEQ ID NO: 25 or by an amino acid sequence having at least 90% identity to an amino acid sequence shown in SEQ ID NO: 25; and/or
(ii) the polypeptide comprises an amino acid sequence shown in any one of SEQ ID NOs: 27, 29, 31 or 33 or an amino acid sequence having at least 90% identity to an amino acid sequence shown in any one of SEQ ID NOs: 27, 29, 31 or 33.

82. The siglec-6-binding polypeptide according to claim 81, wherein:
(a) the extracellular ligand binding domain comprises a spacer domain, such as spacer domain from CD8a, IgG3 or IgG4;
(b) said transmembrane domain comprises a CD28 transmembrane domain, preferably represented by an amino acid sequence shown in SEQ ID NO: 13 or by an amino acid sequence having at least 90% identity to an amino acid sequence shown in SEQ ID NO: 13;
(c) said intracellular signalling domain comprises a costimulatory domain and a CD3 zeta domain, wherein the CD3 zeta domain is preferably represented by an amino acid sequence shown in SEQ ID NO: 19 or by an amino acid sequence having at least 90% identity to an amino acid sequence shown in SEQ ID NO: 19, and wherein the costimulatory domain is preferably:
a CD28 cytoplasmic domain, wherein the CD28 cytoplasmic domain is preferably represented by an amino acid sequence shown in SEQ ID NO: 15 or by an amino acid sequence having at least 90% identity to an amino acid sequence shown in SEQ ID NO: 15, or
a 4-1BB costimulatory domain, wherein the 4-1BB costimulatory domain is preferably represented by an amino acid sequence shown in SEQ ID NO: 17 or by an amino acid sequence having at least 90% identity to an amino acid sequence shown in SEQ ID NO: 17.

83. A polynucleotide or set of polynucleotides encoding the siglec-6-binding polypeptide according to claim 81, wherein preferably,
the polynucleotide comprises a nucleotide sequence represented by SEQ ID NO: 26 or a nucleotide sequence having at least 80% identity to nucleotide sequence shown in SEQ ID NO: 26, and/or
the polynucleotide comprises a nucleotide sequence represented by any one of SEQ ID NO: 28, 30, 32 or 34, or a nucleotide sequence having at least 80% identity to nucleotide sequence shown in any one of SEQ ID NO: 28, 30, 32 or 34.

84. The polynucleotide according to claim 83, wherein the polynucleotide further comprises flanking segments in 5′-direction and in 3′-direction of the polynucleotide encoding the polypeptide, wherein preferably, the flanking segment in 5′-direction is a left inverted repeat/direct repeat (IR/DR) segment and the flanking segment in 3′-direction is a right inverted repeat/direct repeat (IR/DR) segment, and wherein more preferably, the left IR/DR segment is represented by SEQ ID NO: 43 and right IR/DR segment is represented by SEQ ID NO: 44.

85. The polynucleotide according to claim 83, wherein the polynucleotide comprises a nucleotide sequence of a left IR/DR, a polynucleotide sequence encoding the siglec-6-binding polypeptide and a nucleotide sequence of a right IR/DR.

86. An expression vector comprising a polynucleotide or set of polynucleotides according to claim 83, wherein the expression vector is preferably a non-viral vector or a viral vector.

87. The expression vector according to claim 86 that is a non-viral vector, wherein the expression vector is a minimal DNA expression cassette, preferably a minicircle DNA, and/or wherein the expression vector is a transposon donor DNA molecule, wherein the transposon donor DNA molecule is preferably a Sleeping Beauty or PiggyBac transposon donor DNA molecule.

88. The expression vector according to claim 86 that is a viral vector that is a lentiviral or gamma-retroviral vector.

89. An immune cell, wherein said immune cell is preferably a human cell, the immune cell comprising a siglec-6-binding polypeptide according to claim 81 and/or a polynucleotide or set of polynucleotides encoding the same and/or an expression vector comprising the polynucleotide or set of polynucleotides, the immune cell optionally further expressing a detectable marker.

90. The immune cell according to claim 89, wherein the polynucleotide or set of polynucleotides and/or the vector is expressed.

91. The immune cell according to claim 89, wherein said immune cell is a lymphocyte, preferably a T cell or an NK cell, wherein said T cell is preferably a CD4⁺ cell or a CD8⁺ cell.

92. A method for producing (recombinant) immune cells, comprising the steps of
(a) isolating immune cells from a blood sample of a subject, wherein the subject is preferably a human,
(b) transforming or transducing the immune cells with a polynucleotide according to claim 83 or an expression vector comprising the same, and
(c) optionally purifying the transformed or transduced immune cells.

93. The method according to claim 92, wherein in step (b) the immune cells are transformed using 1) a transposable element comprising the polynucleotide and 2) a polynucleotide encoding a transposase, wherein the transposase is preferably Sleeping Beauty transposase, wherein the Sleeping Beauty transposase is preferably represented by an amino acid sequence shown in SEQ ID NO: 45, or wherein the transposase is PiggyBac transposase, and wherein the transposable element is preferably integrated into the genome of the immune cells by the action of the transposase.

94. The method according to claim 92, wherein the immune cell is a lymphocyte, wherein the lymphocyte is preferably a T cell or an NK cell, and wherein the T cell is preferably a CD4⁺ cell or a CD8+cell.

95. An immune cell obtainable by the method of claim 92.

96. A pharmaceutical composition comprising a plurality of immune cells according to claim 89, wherein the plurality of immune cells is optionally a mixture of CD4⁺ and CD8⁺ cells.

97. A method of treatment comprising administering an immune cell according to claim 89 or a pharmaceutical composition comprising the same to a subject.

98. The method of claim 97, wherein the patient has cancer and the cell is administered preferably intravenously.

99. The method of claim 97, wherein the immune cell is a lymphocyte, wherein said lymphocyte is preferably a T cell or an NK cell, wherein the T cell is preferably a CD4⁺ T cell and/or CD8⁺ T cell.

100. The method of claim 97, wherein said cancer is
(a) a siglec-6 expressing cancer;
(b) said cancer is leukemia, and/or
(c) said cancer is primary acute myeloid leukemia (AML), chronic lymphocytic leukemia (CLL), MALT lymphoma or clonal mast cell disease.

101. The method of claim 97, wherein:
i) the method of treating cancer involves the elimination of cancer stem cells of said cancer by said immune cells, wherein the cancer stem cells are preferably CD45^{dim} cells, more preferably CD45^{dim}CD34⁺ cells, and most preferably CD45^{dim}CD34⁺CD38⁻cells, the method optionally further comprising monitoring the elimination of said cancer stem cells;
ii) the method of treating cancer does not involve the elimination of non-cancerous hematopoietic stem or progenitor cells by said immune cells, the method optionally further comprising monitoring the elimination of said non-cancerous hematopoietic stem or progenitor cells;
iii) the method of treating cancer does not involve allogeneic hematopoietic stem cell transplantation, or wherein the subject is a subject having a relapse of the cancer after allogeneic hematopoietic stem cell transplantation;
iv) the method does not involve additional chemotherapy after administration of the immune cells or the pharmaceutical composition and/or after the termination of the therapy with the immune cells or the pharmaceutical composition;
v) the method of treating cancer does not involve depletion of said immune cells after treatment;
vi) the method comprises:
1) determining the expression level of siglec-6 on cancer cells obtained from the subject; followed by 2) administering the immune cell or pharmaceutical composition to the subject,
wherein the immune cell or pharmaceutical composition is preferably administered in step 2) only if siglec-6 is expressed on said cancer cells;
vii) the method involves additional therapy with
(i) a CD70-binding polypeptide that comprises or consists of an antibody or a fragment thereof binding CD70 or that comprises or consists of a chimeric antigen receptor (CAR), or
(ii) an immune cell comprising a CD70-binding polypeptide according to (i) and/or a polynucleotide or set of polynucleotides encoding a CD70-binding polypeptide according to (i) and/or an expression vector comprising a polynucleotide or set of polynucleotides encoding a CD70-binding polypeptide according to (i),
said immune cell being preferably a T-cell such as a CD4⁺ T-cell or CD8⁺-T-cell or an NK-cell, wherein the CD70-binding polypeptide preferably comprises or consists of a chimeric antigen receptor (CAR); and/or
viii) the method involves additional therapy with
(i) a TIM-3-binding polypeptide that comprises or consists of an antibody or a fragment thereof binding TIM-3 or that comprises or consists of a chimeric antigen receptor (CAR), or
(ii) an immune cell comprising a TIM-3-binding polypeptide according to (i) and/or a polynucleotide or set of polynucleotides encoding a TIM-3-binding polypeptide according to (i) and/or an expression vector comprising a polynucleotide or set of polynucleotides encoding a TIM-3-binding polypeptide according to (i),
said immune cell being preferably a T-cell such as a CD4⁺ T-cell or CD8⁺-T-cell or an NK-cell, wherein the TIM-3-binding polypeptide preferably comprises or consists of a chimeric antigen receptor (CAR).
